SEQUENCE LISTING (12 sequences)

SEQ ID NO: 1 - protein, 153 aa, Artificial Sequence
  IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - DNA, 462 bp, Artificial Sequence
  IL-2
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 3 - protein, 177 aa, Artificial Sequence
  IL-7
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 4 - DNA, 534 bp, Artificial Sequence
  IL-7
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 5 - protein, 162 aa, Artificial Sequence
  IL-15
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - DNA, 489 bp, Artificial Sequence
  IL-15
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 7 - DNA, 588 bp, Artificial Sequence
  WPRE sequence
atcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 8 - DNA, 553 bp, Artificial Sequence
  ET promoter
cgcgagttaataattaccagcgcgggccaaataaataatccgcgaggggcaggtgacgtttgcccagcgcgcgctggtaattattaacctcgcgaatattgattcgaggccgcgattgccgcaatcgcgaggggcaggtgacctttgcccagcgcgcgttcgccccgccccggacggtatcgataagcttaggagcttgggctgcaggtcgagggcactgggaggatgttgagtaagatggaaaactactgatgacccttgcagagacagagtattaggacatgtttgaacaggggccgggcgatcagcaggtagctctagaggatccccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 9 - DNA, 335 bp, Artificial Sequence
  Albumin promoter
ggcatgcttccatgccaaggcccacactgaaatgctcaaatgggagacaaagagattaagctcttatgtaaaatttgctgttttacataactttaatgaatggacaaagtcttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaatgatctacagttattggttaaagaagtatattagagcgagtctttctgcacacagatcacctttcctatcaacccc

SEQ ID NO: 10 - DNA, 23 bp, Artificial Sequence
  miR-142 target sequence
tccataaagtaggaaacactaca

SEQ ID NO: 11 - DNA, 22 bp, Artificial Sequence
  miR-155 target sequence
cccctatcacgattagcattaa

SEQ ID NO: 12 - DNA, 21 bp, Artificial Sequence
  miR-223 target sequence
ggggtatttgacaaactgaca

(19) United States
(12) Patent Application Publication
(10) Pub. No.: US 2024/0000892 A1
(43) Pub. Date: Jan. 4, 2024

(54) Agents and Methods for Increasing Liver Immune Response

(71) Applicant: Ospedale San Raffaele S.r.l., Milan (IT)

(72) Inventors: Luca Guidotti, Milan (IT); Matteo Iannacone, Milan (IT)

(21) Appl. No.: 18/038,140

(22) PCT Filed: Nov. 26, 2021
(86) PCT No.: PCT/EP2021/083225

§ 371 Date: May 22, 2023

(30) Foreign Application Priority Data
Nov. 26, 2020 (GB) 2018657.3

(51) Int. Cl. A61K 38/20; A61K 45/06; A61P 37/04

(52) U.S. Cl. CPC A61K 38/2013; A61K 38/2046; A61K 38/2086; A61K 45/06; A61P 37/04

(57) ABSTRACT

An agent that increases the number ofKupffer cells in a subject, or a nucleotide sequence encoding therefor, for use in a method of therapy by increasing liver immune response.

### FIELD OF THE INVENTION

The present invention relates to agents for use in a method of therapy by increasing liver immune response, for example for use in the treatment or prevention of liver infections or liver tumours. In particular, the invention relates to agents that increase the number of Kupffer cells, particularly the proportion of Type 2 Kupffer cells in relation to Type 1 Kupffer cells.

### BACKGROUND TO THE INVENTION

Hepatitis infections, such as hepatitis B virus (HBV) infection, remain a major public health issue worldwide. For example, it has been estimated that about 248 million individuals were positive for hepatitis B surface antigen, a marker of chronic HBV infection, globally in 2010.

Chronic HBV infection is typically acquired at birth or in early childhood, and is particularly prevalent in Asian and African countries where HBV is endemic. The risk of developing chronic infection after exposure drops from ca. 90% in neonates to 1-5% in healthy adults. However, 25% of people who acquire HBV as children will develop primary liver cancer or cirrhosis as adults.

HBV is a non-cytopathic virus that replicates exclusively in hepatocytes without inducing innate immune activation. The outcome of HBV infection is mainly determined by the kinetics, breadth, vigour and effector functions of HBV-specific CD8⁺ T cell responses. CD8⁺ T cell responses to pathogens that exclusively replicate in hepatocytes, such as HBV, are known to vary from severe dysfunction to full differentiation into effector cells endowed with antiviral potential.

CD8⁺ T cells have a critical role in eliminating intracellular pathogens and tumours. In order to exert their defensive function, naïve CD8⁺ T cells need to recognise antigen (Ag), become activated, proliferate and differentiate into effector cells. This process—known as “priming”—occurs preferentially in secondary lymphoid organs, where the specialised microenvironment favours the encounter between naïve CD8⁺ T cells and professional Ag-presenting cells. Indeed, naïve CD8⁺ T cells constantly recirculate between blood and secondary lymphoid organs, while they are prevented from interacting with epithelial cells of non-lymphoid organs by the endothelial barrier.

The liver is an exception to this: the unique anatomy, slow blood flow, presence of endothelial fenestrations and absence of a basement membrane allow CD8⁺ T cells to sense MHC-Ag complexes and other surface ligands on hepatocytes. While priming of CD8⁺ T cells in secondary lymphoid organs has been well characterised, the mechanisms and consequences of intrahepatic priming are less clear. In general, the liver is thought to be biased towards inducing a state of T cell unresponsiveness or dysfunction. This phenomenon underpins the acceptance of liver allografts across complete MHC mismatch barriers, the unresponsiveness toward antigens specifically expressed in hepatocytes, and the propensity of some hepatotropic viruses, such as HBV, to establish persistent infections.

Liver tolerance involves a complex array of coordinated events that ultimately hinder the effector functions of intrahepatic lymphocytes. The unique anatomy and haemodynamics of the fenestrated and basement membrane-less liver capillaries (i.e. sinusoids)—through which about one third of all blood cells transit slowly every minute—allow circulating, intravascular T cells to sense MHC-antigen (Ag) complexes displayed by the non-professional Ag-presenting hepatocytes. Hepatocellular priming of virus-specific naïve CD8⁺ T cells induces local activation and initial vigorous proliferation, but eventually leads to the development of dysfunctional cells devoid of cytotoxic and antiviral activity. The transcriptional signature of these cells is not obviously overlapping with that of other known dysfunctional CD8⁺ T cell states such as exhaustion and, accordingly, CD8⁺ T cells primed by hepatocytes are not readily responsive to in vivo anti-PD-L1 treatment. In vivo IL-2 administration overcomes this dysfunction, illustrating that efficient hepatocellular priming can occur under specific conditions.

While the tolerogenic property of the liver has long been known, the mechanisms underlying this phenomenon, particularly in the context of HBV pathogenesis, are incompletely understood.

Current treatment for chronic HBV infection mainly relies on direct acting antiviral (DAA) drugs (e.g. tenofovir, lamivudine, adefovir, entecavir or telbivudine), which suppress virus production, but do not eradicate HBV from the liver. Accordingly, this leads to a requirement for lifelong treatment. Alternatively, some patients receive a therapy based on pegylated interferon-α (PEG-IFN-α), which whilst having limited treatment duration, has greater adverse effects.

Accordingly, there remains a significant need for improved treatments liver diseases, such as liver infections and tumours, in particular chronic HBV infections.

### SUMMARY OF THE INVENTION

The inventors have surprisingly found that administration of GM-CSF inhibitors enables reinvigoration and restoration of effector responses in dysfunctional CD8⁺ T cells, such as against antigens specifically expressed in hepatocytes. In particular, the inventors found that GM-CSF inhibitors are able to increase effector responses against hepatotropic viruses, such as HBV. The inventors also found that GM-CSF inhibitors are able to increase effector responses in T cells from immune tolerant patients.

Moreover, the inventors' studies have revealed that local administration of GM-CSF inhibitors to the liver is able to increase the effector responses and overcome the tolerogenic potential of the hepatic microenvironment. While not wishing to be bound by theory, the inventors believe inhibition of GM-CSF increases the relative proportion of a subset of Kupffer cells (Type 2 Kupffer cells, KC2) in relation to a different subset (Type 1 Kupffer cells, KC1), and that Type 2 Kupffer cells may play an important role in T cell immunity in the liver.

The inventors also surprisingly found that the co-administration of agents that inhibit GM-CSF and interleukins that bind the IL2 receptor (IL-2R) have a synergistic effect in boosting T cell immunity in the liver. This is noteworthy considering that steady-state KC cross-presentation of HBV Ags is a remarkably inefficient process that cannot be increased by liver inflammation, hepatocellular death or by the administration of therapeutic monoclonal antibodies directed against HBsAg leading to the generation of circulating immune complexes.

In one aspect, the invention provides an agent that increases the number of Kupffer cells in a subject, or a nucleotide sequence encoding therefor, for use in a method of therapy by increasing liver immune response.

In preferred embodiments, the agent increases the number of Type 2 Kupffer cells (KC2). In some embodiments, the agent increases the proportion of Type 2 Kupffer cells (KC2) in relation to Type 1 Kupffer cells (KC1).

In another aspect the invention provides an agent that increases the proportion of Type 2 Kupffer cells (KC2) in relation to Type 1 Kupffer cells (KC1) in a subject, or a nucleotide sequence encoding therefor, for use in a method of therapy by increasing liver immune response.

In some embodiments, the agent is administered simultaneously, sequentially or separately in combination with an interleukin that binds to IL-2 receptor (IL-2R), or a nucleotide sequence encoding therefor.

In another aspect, the invention provides an interleukin that binds to IL-2 receptor (IL-2R), or a nucleotide sequence encoding therefor, for use in a method of therapy by increasing liver immune response, wherein the interleukin is administered simultaneously, sequentially, or separately in combination with an agent that increases the number of Kupffer cells in a subject, or a nucleotide sequence encoding therefor.

In preferred embodiments, the agent increases the number of Type 2 Kupffer cells (KC2). In some embodiments, the agent increases the proportion of Type 2 Kupffer cells (KC2) in relation to Type 1 Kupffer cells (KC1).

In another aspect, the invention provides an interleukin that binds to IL-2 receptor (IL-2R), or a nucleotide sequence encoding therefor, for use in a method of therapy by increasing liver immune response, wherein the interleukin is administered simultaneously, sequentially, or separately in combination with an agent that increases the proportion of Type 2 Kupffer cells (KC2) in relation to Type 1 Kupffer cells (KC1) in a subject, or a nucleotide sequence encoding therefor.

In preferred embodiments, the agent is a granulocyte-macrophage colony-stimulating factor (GM-CSF) inhibitor.

In another aspect, the invention provides a granulocyte-macrophage colony-stimulating factor (GM-CSF) inhibitor, or a nucleotide sequence encoding therefor, for use in a method of therapy by increasing liver immune response.

In another aspect, the invention provides an interleukin that binds to IL-2 receptor (IL-2R), or a nucleotide sequence encoding therefor, for use in a method of therapy by increasing liver immune response, wherein the interleukin is administered simultaneously, sequentially, or separately in combination with a granulocyte-macrophage colony-stimulating factor (GM-CSF) inhibitor, or a nucleotide sequence encoding therefor.

In some embodiments, the method of therapy is treatment or prevention of a liver infection. In some embodiments, the method of therapy is treatment or prevention of a primary liver tumour. In some embodiments, the method of therapy is treatment or prevention of a secondary liver tumour.

In another aspect, the invention provides an agent that increases the number of Kupffer cells in a subject, or a nucleotide sequence encoding therefor, for use in treatment or prevention of a liver infection. In preferred embodiments, the agent increases the number of Type 2 Kupffer cells (KC2). In some embodiments, the agent increases the proportion of Type 2 Kupffer cells (KC2) in relation to Type 1 Kupffer cells (KC1).

In another aspect the invention provides an agent that increases the proportion of Type 2 Kupffer cells (KC2) in relation to Type 1 Kupffer cells (KC1) in a subject, or a nucleotide sequence encoding therefor, for use in treatment or prevention of a liver infection. In another aspect, the invention provides a granulocyte-macrophage colony-stimulating factor (GM-CSF) inhibitor, or a nucleotide sequence encoding therefor, for use in treatment or prevention of a liver infection.

In another aspect, the invention provides an agent that increases the number of Kupffer cells in a subject, or a nucleotide sequence encoding therefor, for use in treatment or prevention of a primary or secondary liver tumour. In preferred embodiments, the agent increases the number of Type 2 Kupffer cells (KC2). In some embodiments, the agent increases the proportion of Type 2 Kupffer cells (KC2) in relation to Type 1 Kupffer cells (KC1).

In another aspect the invention provides an agent that increases the proportion of Type 2 Kupffer cells (KC2) in relation to Type 1 Kupffer cells (KC1) in a subject, or a nucleotide sequence encoding therefor, for use in treatment or prevention of a primary or secondary liver tumour. In another aspect, the invention provides a granulocyte-macrophage colony-stimulating factor (GM-CSF) inhibitor, or a nucleotide sequence encoding therefor, for use in treatment or prevention of a primary or secondary liver tumour.

In another aspect, the invention provides an interleukin that binds to IL-2 receptor (IL-2R), or a nucleotide sequence encoding therefor, for use in treatment or prevention of a liver infection, wherein the interleukin is administered simultaneously, sequentially, or separately in combination with an agent that increases the number of Kupffer cells in a subject, or a nucleotide sequence encoding therefor. In preferred embodiments, the agent increases the number of Type 2 Kupffer cells (KC2). In some embodiments, the agent increases the proportion of Type 2 Kupffer cells (KC2) in relation to Type 1 Kupffer cells (KC1).

In another aspect, the invention provides an interleukin that binds to IL-2 receptor (IL-2R), or a nucleotide sequence encoding therefor, for use in treatment or prevention of a liver infection, wherein the interleukin is administered simultaneously, sequentially, or separately in combination with an agent that increases the proportion of Type 2 Kupffer cells (KC2) in relation to Type 1 Kupffer cells (KC1) in a subject, or a nucleotide sequence encoding therefor.

In another aspect, the invention provides an interleukin that binds to IL-2 receptor (IL-2R), or a nucleotide sequence encoding therefor, for use in treatment or prevention of a primary or secondary liver tumour, wherein the interleukin is administered simultaneously, sequentially, or separately in combination with an agent that increases the number of Kupffer cells in a subject, or a nucleotide sequence encoding therefor. In preferred embodiments, the agent increases the number of Type 2 Kupffer cells (KC2). In some embodiments, the agent increases the proportion of Type 2 Kupffer cells (KC2) in relation to Type 1 Kupffer cells (KC1).

In another aspect, the invention provides an interleukin that binds to IL-2 receptor (IL-2R), or a nucleotide sequence encoding therefor, for use in treatment or prevention of a primary or secondary liver tumour, wherein the interleukin is administered simultaneously, sequentially, or separately in combination with an agent that increases the proportion of Type 2 Kupffer cells (KC2) in relation to Type 1 Kupffer cells (KC1) in a subject, or a nucleotide sequence encoding therefor.

In some embodiments, the liver infection is a viral liver infection.

In some embodiments, the liver infection is a Plasmodium infection, for example a Plasmodium falciparum, Plasmodium vivax, Plasmodium ovale, Plasmodium malariae or Plasmodium knowlesi infection. In some embodiments, the method of therapy is treatment or prevention of malaria.

In some embodiments, the primary liver tumour is a hepatocellular carcinoma.

In some embodiments, the secondary liver tumour is a metastasis.

In some embodiments, the liver infection is a hepatitis virus infection. In some embodiments, the liver infection is a chronic hepatitis virus infection.

In some embodiments, the liver infection is a hepatitis B virus (HBV) infection. In some embodiments, the liver infection is a hepatitis C virus (HCV) infection.

In some embodiments, the GM-CSF inhibitor decreases the activity of GM-CSF or GM-CSF Receptor (GM-CSF-R), preferably GM-CSF.

In some embodiments, the GM-CSF inhibitor is an antibody or a fragment thereof that binds GM-CSF or GM-CSF Receptor (GM-CSF-R), preferably GM-CSF. In some embodiments, the antibody or fragment thereof depletes GM-CSF or GM-CSF-R.

In some embodiments, the GM-CSF inhibitor is a GM-CSF Receptor (GM-CSF-R) antagonist.

In some embodiments, the antibody is a monoclonal antibody, a humanised antibody, a single-chain antibody or an antibody fragment.

In some embodiments, the use further comprises administration with a chemotherapeutic agent. In some embodiments, the antibody is conjugated to said chemotherapeutic agent.

In some embodiments, the GM-CSF inhibitor reduces expression of GM-CSF or GM-CSF Receptor (GM-CSF-R), preferably GM-CSF.

In some embodiments, the GM-CSF inhibitor is selected from a group consisting of an shRNA, siRNA, miRNA or antisense DNA/RNA.

In some embodiments, the interleukin is selected from the group consisting of IL-2, IL-7 or IL-15.

In preferred embodiments, the interleukin is IL-2. In some embodiments, the interleukin is IL-7. In some embodiments, the interleukin is IL-15.

In some embodiments, the agent, interleukin and/or nucleotide sequence(s) encoding therefor is adapted to be targeted to the liver.

In some embodiments, the agent, interleukin and/or nucleotide sequence(s) encoding therefor is adapted to be targeted to Type 2 Kupffer cells (KC2). In preferred embodiments, the interleukin and/or nucleotide sequence encoding therefor is adapted to be targeted to Type 2 Kupffer cells (KC2).

In some embodiments, the agent, interleukin and/or nucleotide sequence(s) encoding therefor is comprised in a nanoparticle. In preferred embodiments, the nanoparticle comprises a liver-specific ligand.

In some embodiments, the nanoparticle is a polymeric nanoparticle, inorganic nanoparticle or lipid nanoparticle.

In preferred embodiments, the nanoparticle is a liposome.

In some embodiments, the nucleotide sequence(s) encoding the agent and/or interleukin is in the form of one or more vectors. In preferred embodiments, the vector(s) is adapted for liver-specific expression of the nucleotide sequence(s).

In some embodiments, the agent, interleukin and/or nucleotide sequence(s) encoding therefor is adapted to be targeted to hepatocytes. In some embodiments, the agent, interleukin and/or nucleotide sequence(s) encoding therefor is adapted to be targeted to liver sinusoidal endothelial cells. In some embodiments, the agent, interleukin and/or nucleotide sequence(s) encoding therefor is adapted to be targeted to Kupffer cells.

In some embodiments, the nucleotide sequence encoding the agent is in the form of a vector. In some embodiments, the nucleotide sequence encoding the interleukin is in the form of a vector. In some embodiments, the nucleotide sequences encoding the agent and interleukin are comprised in a vector.

In some embodiments, the nucleotide sequence encoding the agent is in the form of a vector adapted for liver-specific expression of the nucleotide sequence. In some embodiments, the nucleotide sequence encoding the interleukin is in the form of a vector adapted for liver-specific expression of the nucleotide sequence. In some embodiments, the nucleotide sequences encoding the agent and interleukin are comprised in a vector adapted for liver-specific expression of the nucleotide sequences.

In some embodiments, the nucleotide sequence encoding the agent is operably linked an expression control sequence for liver-specific expression. In some embodiments, the nucleotide sequence encoding the interleukin is operably linked to an expression control sequences for liver-specific expression. In some embodiments, the nucleotide sequences encoding the agent and the interleukin are operably linked to one or more expression control sequences for liver-specific expression.

In some embodiments, the liver-specific expression is hepatocyte-specific expression. In some embodiments, the liver-specific expression is liver sinusoidal endothelial cell-specific expression. In some embodiments, the liver-specific expression is Kupffer cell-specific expression.

In some embodiments, the expression control sequence is a liver-specific promoter and/or enhancer.

In some embodiments, the nucleotide sequence encoding the agent is operably linked to one or more miR-142, miR-155 and/or miR-223 target sequences, preferably one or more miR-142 target sequences. In some embodiments, the nucleotide sequence the interleukin is operably linked to one or more miR-142, miR-155 and/or miR-223 target sequences, preferably one or more miR-142 target sequences. In some embodiments, the nucleotide sequences encoding the agent and interleukin are operably linked to one or more miR-142, miR-155 and/or miR-223 target sequences, preferably one or more miR-142 target sequences.

In some embodiments, the one or more vector(s) comprises two, three or four miR-142, miR-155 and/or miR-223 target sequences operably linked to the nucleotide sequence(s) encoding the agent and/or interleukin.

In some embodiments, the one or more vector(s) comprises a liver-specific promoter and/or enhancer operably linked to the nucleotide sequence(s) encoding the agent and/or interleukin.

In some embodiments, the liver-specific promoter and/or enhancer is an hepatocyte-specific promoter and/or enhancer.

In some embodiments, the hepatocyte-specific promoter is selected from the group consisting of an ET promoter, albumin promoter, transthyretin promoter, alpha1-antitrypsin promoter and apoE/alpha1-antitrypsin promoter. In preferred embodiments, the hepatocyte-specific promoter is an ET promoter.

In some embodiments, the one or more vector(s) comprises a liver sinusoidal endothelial cell-specific promoter and/or enhancer operably linked to the nucleotide sequence(s) encoding the agent and/or interleukin.

In some embodiments, the liver sinusoidal endothelial cell-specific promoter is selected from the group consisting of a vascular endothelial cadherin (VEC) promoter, intercellular adhesion molecule 2 (ICAM2) promoter, foetal liver kinase 1 (Flk1) promoter and Tie2 promoter.

In some embodiments, the one or more vector(s) comprises a Kupffer cell-specific promoter and/or enhancer operably linked to the nucleotide sequence(s) encoding the agent and/or interleukin.

In some embodiments, the Kupffer cell-specific promoter is a CD11b promoter.

In some embodiments, the one or more vector(s) comprises one or more liver- or hepatocyte-specific cis-acting regulator modules (CRMs, see Merlin, S. et al. (2019) Molecular Therapy: Methods & Clinical Development 12: 223-232), for example CRM8.

In some embodiments, the nucleotide sequence encoding the interleukin (preferably IL-2) is in the form of an mRNA and is comprised in a nanoparticle. Preferably, the nucleotide sequence encoding the interleukin is operably linked to one or more miRNA target sequence. In preferred embodiments, the nucleotide sequence encoding the interleukin is operably linked to one or more miR-142, miR-155 and/or miR-223 target sequence, preferably one or more miR-142 target sequence. In preferred embodiments, the nanoparticle comprises a liver-specific ligand. In some embodiments, the nanoparticle is a polymeric nanoparticle, inorganic nanoparticle or lipid nanoparticle. In preferred embodiments, the nanoparticle is a liposome.

In some embodiments, the vector is a viral vector. In some embodiments, the vector is an RNA vector.

In some embodiments, the vector is a retroviral, lentiviral, adenoviral, adeno-associated viral (AAV) or arenaviral vector. In some embodiments, the vector is a lentiviral vector. In some embodiments, the vector is a replication-deficient lymphocytic choriomeningitis viral vector.

In some embodiments, the vector is in the form of a viral vector particle.

In some embodiments, the viral vector particle comprises (e.g. overexpresses) CD47 (e.g. as described in U.S. Pat. No. 9,050,269). In some embodiments, the viral vector particle does not comprise or substantially does not comprise MHC-I, preferably surface-exposed MHC-I. Preferably, the viral vector particle is substantially devoid of surface-exposed MHC-I molecules. In some embodiments, the viral vector particle comprises (e.g. overexpresses) CD47 and does not comprise or substantially does not comprise MHC-I, preferably surface-exposed MHC-I.

In some embodiments, the viral vector comprises an envelope protein or capsid protein for liver cell-specific transduction. In some embodiments, the viral vector comprises an envelope protein or capsid protein for hepatocyte-specific transduction. In some embodiments, the viral vector comprises an envelope protein or capsid protein for liver sinusoidal endothelial cell-specific transduction. In some embodiments, the viral vector comprises an envelope protein or capsid protein for Kupffer cell-specific transduction.

In some embodiments, the viral vector (e.g. lentiviral vector) comprises a GP64 or hepatitis B virus envelope protein. GP64 or hepatitis B virus envelope proteins may give rise to hepatocyte-specific transduction.

In some embodiments, the vector is in the form a liposome, optionally wherein the vector is an RNA vector.

In some embodiments, the agent and/or interleukin, or nucleotide sequence(s) encoding therefor, is administered intravenously.

In some embodiments, the agent and/or interleukin, or nucleotide sequence(s) encoding therefor, is locally administered to a subject, optionally to a subject's liver.

In some embodiments, the agent and/or interleukin, or nucleotide sequence(s) encoding therefor, is administered as part of an adoptive T cell therapy.

In some embodiments, the agent and/or interleukin, or nucleotide sequence(s) encoding therefor, is administered simultaneously, separately or sequentially with a population of T cells. In some embodiments, the T cells express a chimeric antigen receptor (CAR) or a T cell receptor (TCR). In some embodiments, the CAR or TCR binds to a hepatitis virus antigen.

In another aspect, the invention provides a product comprising: (a) an agent that increases the number of Kupffer cells in a subject, or a nucleotide sequence encoding therefor; and (b) an interleukin that binds to IL-2 receptor (IL-2R), or nucleotide sequence encoding therefor, optionally wherein the product is a kit or a composition.

In another aspect, the invention provides a product comprising: (a) an agent that increases the number of Kupffer cells in a subject, or a nucleotide sequence encoding therefor; and (b) a population of T cells, optionally wherein the T cells express a chimeric antigen receptor (CAR) or a T cell receptor (TCR), optionally wherein the product is a kit or a composition.

In another aspect, the invention provides a product comprising: (a) an agent that increases the proportion of Type 2 Kupffer cells (KC2) in relation to Type 1 Kupffer cells (KC1) in a subject, or a nucleotide sequence encoding therefor; and (b) an interleukin that binds to IL-2 receptor (IL-2R), or nucleotide sequence encoding therefor, optionally wherein the product is a kit or a composition.

In another aspect, the invention provides a product comprising: (a) an agent that increases the proportion of Type 2 Kupffer cells (KC2) in relation to Type 1 Kupffer cells (KC1) in a subject, or a nucleotide sequence encoding therefor; and (b) a population of T cells, optionally wherein the T cells express a chimeric antigen receptor (CAR) or a T cell receptor (TCR), optionally wherein the product is a kit or a composition.

In another aspect, the invention provides a product comprising: (a) a granulocyte-macrophage colony-stimulating factor (GM-CSF) inhibitor, or a nucleotide sequence encoding therefor; and (b) an interleukin that binds to IL-2 receptor (IL-2R), or nucleotide sequence encoding therefor, optionally wherein the product is a kit or a composition.

In another aspect, the invention provides a product comprising: (a) a granulocyte-macrophage colony-stimulating factor (GM-CSF) inhibitor, or a nucleotide sequence encoding therefor; and (b) a population of T cells, optionally wherein the T cells express a chimeric antigen receptor (CAR) or a T cell receptor (TCR), optionally wherein the product is a kit or a composition.

In some embodiments, the product is a pharmaceutical composition further comprising a pharmaceutically-acceptable carrier, diluent or excipient.

In some embodiments, the product further comprises a population of T cells, optionally wherein the T cells express a chimeric antigen receptor (CAR) or a T cell Receptor (TCR).

In some embodiments, the pharmaceutical composition further comprises a population of T cells, optionally wherein the T cells express a chimeric antigen receptor (CAR) or a T cell Receptor (TCR).

In some embodiments, the CAR or TCR binds to a hepatitis virus antigen. In some embodiments, the hepatitis virus antigen is selected from the group consisting of hepatitis B virus large envelope protein; hepatitis B virus middle envelope protein; hepatitis B virus small envelope protein; hepatitis B virus core protein; and hepatitis B virus polymerase.

In another aspect, the invention provides a method of treatment comprising administering an agent that increases the number of Kupffer cells in a subject, or a nucleotide sequence encoding therefor, to a subject in need thereof, wherein liver immune response is increased in the subject.

In another aspect the invention provides a method of treatment comprising administering an agent that increases the proportion of Type 2 Kupffer cells (KC2) in relation to Type 1 Kupffer cells (KC1) in a subject, or a nucleotide sequence encoding therefor, to a subject in need thereof, wherein liver immune response is increased in the subject.

In another aspect, the invention provides a method of treating a liver infection, a primary liver tumour or a secondary liver tumour comprising administering an agent that increases the number of Kupffer cells in a subject, or a nucleotide sequence encoding therefor, to a subject in need thereof.

In another aspect the invention provides a method of treating a liver infection, a primary liver tumour or a secondary liver tumour comprising administering an agent that increases the proportion of Type 2 Kupffer cells (KC2) in relation to Type 1 Kupffer cells (KC1) in a subject, or a nucleotide sequence encoding therefor, to a subject in need thereof.

In some embodiments, the agent is administered simultaneously, sequentially or separately in combination with an interleukin that binds to IL-2 receptor (IL-2R), or a nucleotide sequence encoding therefor.

In preferred embodiments, the agent is a granulocyte-macrophage colony-stimulating factor (GM-CSF) inhibitor.

In another aspect, the invention provides a method of treatment comprising administering a granulocyte-macrophage colony-stimulating factor (GM-CSF) inhibitor, or a nucleotide sequence encoding therefor, to a subject in need thereof, wherein liver immune response is increased in the subject.

In some embodiments, the method of treatment is treatment of a liver infection, a primary liver tumour or a secondary liver tumour.

In another aspect, the invention provides a method of treating a liver infection, a primary liver tumour or a secondary liver tumour comprising administering a granulocyte-macrophage colony-stimulating factor (GM-CSF) inhibitor, or a nucleotide sequence encoding therefor, to a subject in need thereof, wherein liver immune response is increased in the subject.

### DESCRIPTION OF THE DRAWINGS

FIG. 1. KCs are required for optimal in vivo reinvigoration of intrahepatically-primed T cells by II-2.

(A) Schematic representation of the experimental setup. 5×10⁶ Cor93 and Env28 T_{N} were transferred into C57BL/6×Balb/c F1 (WT) or MUP-core×Balb/c F1 (MUP-core) recipients. When indicated, mice were injected with 2.5×10⁵ infectious units of non-replicating rLCMV-core/env 4 h prior to T_{N} transfer. Selected MUP-core mice received clodronate liposomes (CLL) and/or IL-2/anti-IL-2 complexes (IL-2c) at the indicated timepoints. Livers were collected and analyzed five days after T_{N} transfer. (B) Representative confocal immunofluorescence micrographs of liver sections from the indicated mice 48 h after CLL treatment. KCs were identified as F4/80⁺ cells and are depicted in red. Sinusoids were identified as Lyve-1⁺ cells and are depicted in grey. Scale bars represent 100 μm. (C-D) Representative flow cytometry plot (C) and absolute numbers (D) of KCs from the indicated mice 48 h after CLL treatment. KCs were identified as live, CD45⁺, TIM4⁺, F4/80⁺ cells. n=3 *p value<0.05, one tailed Mann-Whitney U-test. (E) Absolute numbers of dendritic cells (DCs, identified as live, MHC-II^{high}, CD11c⁺ cells) from the indicated mice 48 h after CLL treatment. n=3. (F-G) Total numbers (F) and numbers of IFN-γ-producing (G) Cor93 and Env28 T cells in the livers of indicated mice. n=4. *p value<0.05, ** p value<0.01, *** p value<0.001, one-way Brown-Forsythe and Welch ANOVA test with Dunnett correction for multiple comparison. Each group was compared to control. Normal distribution was verified by Shapiro-Wilk test. (H) Representative confocal immunofluorescence micrographs of liver sections from the indicated mice five days after T_{N} transfer. Cor93 T cells were identified as GFP⁺ cells and are depicted in green. Env28 T cells were identified as DsRed⁺ cells and are depicted in red. Sinusoids were identified as Lyve-1⁺ cells and are depicted in grey. Scale bars represent 100 μm. (I) Schematic representation of the experimental setup. MUP-core mice were lethally irradiated and reconstituted with CD11c^{DTR} bone marrow (BM). Eight weeks after BM reconstitution, 1×10⁶ Cor93 T_{N} were transferred. Indicated mice were treated with diphtheria toxin (DT) every 48 h starting from three days before T cell injection. Indicated mice received IL-2c one day after Cor93 T cell transfer. Livers were collected and analyzed five days after T_{N} transfer. (J-K) Representative flow cytometry plot (J) and absolute numbers (K) of DCs (identified as live, MHC-II^{high}, CD11c⁺ cells) from the indicated mice at the time of Cor93 T cell transfer. (PBS, n=3; DT n=4) *p value<0.05, one tailed Mann-Whitney U-test. (L) Representative confocal immunofluorescence micrographs of liver sections from the indicated mice 48 h after DT treatment. KCs were identified as F4/80⁺ cells and are depicted in red. Sinusoids were identified as Lyve-1⁺ cells and are depicted in grey. Scale bars represent 50 μm. (M-N) Representative flow cytometry plot (M) and absolute numbers (N) of KCs (identified as live, CD45⁺, TIM4⁺, F4/80⁺ cells) from the indicated mice at the time of Cor93 T cell transfer. (PBS, n=3; DT n=4) (O-P) Total numbers (O) and numbers of IFN-γ-producing (P) Cor93 T cells in the livers of the indicated mice. n=5. (Q) Representative confocal immunofluorescence micrographs of liver sections from the indicated mice five days after T_{N} transfer. Cor93 T cells were identified as CD45.1⁺ cells and are depicted in green. Sinusoids were identified as Lyve-1⁺ cells and are depicted in grey. Scale bars represent 100 μm.

Data are representative of at least 3 independent experiments.

FIG. 2. KCs respond to IL-2 and cross-present hepatocellular antigens. (A) Representative flow cytometry plots of CD25 (left panel), CD122 (middle panel), and CD132 (right panel) expression on CD45⁺ (blue) and F4/80⁺ (red) cell populations in the livers of C57BL/6 mice. Isotype control is depicted in gray. (B) Mean Fluorescent Intensity (MFI) of CD25 (left), CD122 (middle), CD132 (right) expression on live CD45⁺ (blue) and F4/80⁺ (red) cells in the livers of C57BL/6 mice. (C) Schematic representation of the experimental setup. Liver non-parenchymal cells (LNPCs) were isolated from C57BL/6 mice and incubated in vitro with increasing doses of rIL-2. After 15 minutes pSTAT5 signal was analyzed on CD45⁺ F4/80⁺ TIM4⁺ cells (KCs) or CD31⁺ CD45⁻ cells (LSECs) by flow cytometry (representative plot at the bottom). (D) Fold change of STAT5 phosphorylation upon treatment with the indicated concentrations of rIL-2 in KCs (red dots) or LSECs (blue dots). *** p value<0.001, two-way ANOVA with Geisser-Greenhouse's correction. Significance indicates time×column factor. (E) Western blot analysis of STAT5/pSTAT5 in adherent KCs isolated from C57BLJ6 WT mice and incubated in vitro with IL-2c or PBS. (F) Schematic representation of the experimental setup. C57BLJ6 mice were treated in vivo with PBS or IL-2c. 48 hrs after treatment, liver non-parenchymal cells (LNPCs) were isolated and RNA-seq was performed on FACS-sorted KCs. (G) KC sorting strategy. KCs were identified as live, CD45⁺, Lineage⁻ (CD3, CD19, Ly6G, CD49b), F4/80⁺, CD64⁺, TIM4⁺ cells. (H) Clustering of top significant (EnrichR Combined Score>100, FDR<0.05) Gene Ontology Biological Processes and KEGG pathways of processes up-regulated in KCs upon in vivo IL-2c treatment. The thermal scale represents the Jaccard Similarity Coefficient between every gene set pair (blue representing a 0 Similarity Coefficient, red a 1 Similarity Coefficient). (I) Volcano plot of RNA-Seq results. The X-axis represents the Log 2 Fold-Change of Differentially Expressed Genes (DEG) upon IL-2c treatment, the Y-axis the −Log 10(FDR). Only DEGs with a FDR<0.05 were considered. Genes belonging to specific biological process are highlighted in different colors (see also FIG. 9A-E). (J) Radar plot of different biological processes. Each dimension of the radar plot is represented as the mean of the Transcripts Per kilobase Million (TPM) of selected genes (see also FIG. 9A-E), in PBS (blue) and IL-2c treated (red) samples. Values range from 0 to 350 TPM. (K) Heatmap of genes linked to antigen presentation that were upregulated in KCs upon IL-2c treatment. Values are in Z-score, calculated from scaling by row the Log 2(TPM) values. (L) MFI of H2-Kb, CD40 and CD80 expression on KCs (defined as live, CD45⁺, TIM4⁺, F4/80⁺ cells) 48h after PBS or IL-2c treatment in vivo. * p value<0.05, one tailed Mann-Whitney U-test. (M) Schematic representation of the experimental setup. HBV replication-competent transgenic mice (HBV Tg) were treated in vivo with PBS or IL-2c. After 48 h liver non-parenchymal cells (LNPCs) were isolated, KCs were seeded for 2 h and co-cultured with in vitro-differentiated Cor93 effector T cells (Cor93 T_{E}). After 4 h, T cells were harvested and analyzed by flow cytometry. (N-O) Representative flow cytometry plot (N) and percentage (O) of IFN-g producing Cor93 T_{E} cells in the indicated conditions. ** p value<0.01, one tailed Mann-Whitney U-test. (P) Schematic representation of the experimental setup. C57BL/6 WT mice were treated in vivo with PBS or IL-2c. After 48 h LNPCs were isolated, and KCs were purified by immunomagnetic separation. Purified KCs were co-cultured with CellTrace™ violet (CTV)-labelled Cor93 T_{N}. Serum from HBV replication-competent transgenic mice (containing the indicated concentrations of HBeAg) was added to the wells (note that HBeAg contains the Cor93 determinant). After 4 days, Cor93 T cells were harvested and analyzed by flow cytometry. (Q-R) Representative flow cytometry plots (Q) and percentages (R) of proliferating Cor93 T cells at the indicated conditions. *p value<0.05, ** p value<0.01, one-way Brown-Forsythe and Welch ANOVA test with Dunnett correction for multiple comparison. Each group was compared to every other group within the same antigen dose. Normal distribution was verified by Shapiro-Wilk test. (S) Schematic representation of the experimental setup. MUP-core mice were lethally irradiated and reconstituted with WT or TAP1^{-/-} bone marrow (BM). Eight weeks after BM reconstitution mice received two injection of clodronate liposomes (CLL) to remove residual radio-resistant KCs. Two weeks after the last dose of CLL, 5×10⁶ Cor93 T_{N} were transferred. Indicated mice received IL-2c one day after Cor93 T cell transfer. Livers were collected and analyzed five days after Cor93 T_{N} transfer. (T-U) Total numbers (T) and numbers of IFN-γ-producing (U) Cor93 T cells in the livers of the indicated mice. ** p value<0.01, *** p value<0.001, two-way ANOVA with Sidak's multiple comparison test.

Data are representative of at least 3 independent experiments.

FIG. 3. Identification of a KC subset with enriched II-2 sensing machinery.

(A) Representative flow cytometry plot of KC1/KC2 gating strategy. KC1 are defined as ESAM⁻ CD206⁻ KCs. KC2 are defined as ESAM⁺ CD206⁺ KCs. (B) Relative representation of KC1 and KC2 in the liver of C57BLJ6 mice. (C) Representative confocal immunofluorescence micrographs of liver sections from C57BL/6 mice. Sinusoids were identified as CD38⁺ cells and are depicted in white. CD206⁺ cells are depicted in red, F4/80⁺ cells in green. Scale bars represent 50 μm or 10 μm. (D) GSEA relative to the HALLMARK_IL2_STAT5_SIGNALING Gene Set contained in MSigDB, version 6. Genes were pre-ranked based on the Log 2 Fold Change between KC2 and KC1. (E) Heatmap representing the relative expression of the IL-2 receptor signaling components in KC1 and KC2 isolated from C57BL/6 mice. Values in log 2TPM were scaled by row across samples (Z-score). (F-G) Representative flow cytometry plots (F) and MFI (G) of CD25, CD122 and CD132 expression in KC1, KC2 and LSEC (defined as live, CD45⁻, CD31⁺ cells) in C57BLJ6 mice. * p value<0.05, ** p value<0.01, two-way ANOVA with Sidak's multiple comparison test. (H-J) MFI of H2-Kb (H), CD40 (I) and CD80 (J) expression on KC1 (blue) and KC2 (red) 48 h after PBS or IL-2c treatment in vivo. * p value<0.05, ** p value<0.01, two-way ANOVA with Sidak's multiple comparison test. Test is performed comparing PBS vs IL-2c treatment and KC1 vs KC2. (K) Schematic representation of the experimental setup. HBV Tg mice were injected with 1×10⁶ Cor93 T_{N} cells. Mice were treated with PBS or IL-2c one day after Cor93 T_{N} transfer. Livers were collected and analyzed five days after T_{N} transfer. Representative flow cytometry plots (bottom) of KC1 and KC2 in the livers upon PBS (left) or IL-2c (right) treatment. n=4. (L-N) Ratio between KC1 and KC2 (L) and absolute numbers of KC1 (M) and KC2 (N) in the liver of PBS (blue) or IL-2c (red) treated mice. *p value<0.05, one tailed Mann-Whitney U-test.

Data are representative of at least 3 independent experiments.

FIG. 4. KC2 are required for the optimal restoration of intrahepatically-primed, dysfunctional CD8⁺ T cells by IL-2.

(A) Schematic representation of the experimental setup. MUP-core mice were lethally irradiated and reconstituted with Cdh5^{CreERT2}; Rosa26^{iDTR} bone marrow (BM). Four weeks later mice received two injections of clodronate liposomes (CLL) to remove residual radio-resistant KCs. Nine weeks after BM reconstitution, mice were treated once with 5 mg of Tamoxifen by oral gavage. Mice were treated with diphtheria toxin (DT) every 48 h starting three days before Cor93 T_{N} injection (1×10⁶ cells/mouse). Indicated mice received IL-2c one day after Cor93 T_{N} transfer. Livers were collected and analyzed five days after Cor93 T_{N} transfer. (B) Representative flow cytometry plots of KC1 and KC2 populations gated on total KCs (live, CD45⁺, TIM4⁺, F4/80⁺ cells) in the liver of the indicated mice at the time of T_{N} injection. (C-D) Total numbers (C) and numbers (D) of IFN-γ-producing Cor93 T cells in the livers of the indicated mice. PBS, n=5; DT, n=4. *p value<0.05, two tailed Mann-Whitney U-test. (E) Levels of ALT in the serum of the indicated mice at the indicated timepoints. PBS, n=5; DT, n=5. *** p value<0.001, two-way ANOVA with Sidak's multiple comparison test. (F) Representative confocal immunofluorescence micrographs of liver sections from the indicated mice five days after Cor93 T_{N} transfer. Cor93 T cells were identified as CD45.1⁺ cells and are depicted in green. Sinusoids were identified as CD38⁺ cells and are depicted in gray. Scale bars represent 100 μm. (G) Schematic representation of the experimental setup. HBV Tg mice were injected with 1×10⁶ Cor93 T_{N} cells. Mice were treated with anti-GM-CSF depleting antibody every 48 h starting from one day before T cell transfer. Mice were treated with PBS or IL-2c one day after T_{N} cell transfer. Livers were collected and analyzed five days after T_{N} transfer. PBS, n=4; IL-2c, n=3; IL-2c+anti-GM-CSF, n=4. (H) Representative flow cytometry plots of KC1 and KC2 population in the liver of the indicated mice at the time of T_{N} injection. (I-J) Total numbers (I) and numbers of IFN-γ-producing (J) Cor93 T cells in the livers of the indicated mice. (K) Levels of ALT in the serum of the indicated mice. * p value<0.05, ** p value<0.01, two-tailed Mann-Whitney U-test. (L) Representative confocal immunofluorescence micrographs of liver sections from the indicated mice five days after T_{N} transfer. Cor93 T cells were identified as CD45.1⁺ cells and are depicted in green. Sinusoids were identified as CD38⁺ cells and are depicted in gray. Scale bars represent 100 μm.

Data are representative of at least 3 independent experiments.

FIG. 5. Neutrophils and monocytes are dispensable for T cell reinvigoration by IL-2.

(A) Schematic representation of the experimental setup. 1×10⁶ Cor93 T_{N} were transferred into HBV transgenic (HBV Tg) recipients. Mice were injected with anti-Ly6G depleting antibody or the isotype control one day before and one day after T cell injection. Indicated mice received IL-2c one day after Cor93 T_{N} transfer. Livers were collected and analyzed five days after T cell transfer. (B-C) Numbers of neutrophils (B) and monocytes (C) in the blood in the indicated mice at the time of Cor93 T_{N} injection (Isotype control n=7, anti-Ly6G n=6). **p value<0.01, one tailed Mann-Whitney U-test. (D-E) Total numbers (D) and numbers of IFN-γ-producing (E) Cor93 T cells in the livers of the indicated mice (PBS: isotype control n=3, anti-Ly6G n=4; IL-2c: isotype control n=3, anti-Ly6G n=3). (F) Schematic representation of the experimental setup. 1×10⁶ Cor93 T_{N} were transferred into HBV Tg recipients. Mice were injected with anti-Gr1 depleting antibody or isotype control every 48 h starting 3 days before T cell injection. Indicated mice received IL-2c one day after Cor93 T_{N} cell transfer. Livers were collected and analyzed five days after T cell transfer. (G-H) Numbers of neutrophils (G) and monocytes (H) in the blood of the indicated mice at the time of T cell injection (Isotype control n=8, anti-Gr1 n=8). *** p value<0.001, one-tailed Mann-Whitney U-test. (1-J) Total numbers (I) and numbers of IFN-γ-producing (J) Cor93 T cells in the livers of the indicated mice (PBS: isotype control n=3, anti-Gr1 n=4; IL-2c: isotype control n=3 anti-Gr1, n=3).

Data are representative of at least 3 independent experiments.

FIG. 6. pSTAT5 expression in Tregs upon II-2 treatment.

(A) Schematic representation of the experimental setup. Splenocytes were isolated from C57BL/6 mice and incubated in vitro with increasing concentrations of rIL-2. After fifteen minutes pSTAT5 signal was analyzed on Tregs (identified as live, CD45⁺, CD4⁺, Foxp3⁺ cells) by flow cytometry. (B) Representative flow cytometry plot of pSTAT5 expression in Tregs from mice treated with 1 ng/ml of rIL-2. (C) Levels of phosphorylated STAT5 in Tregs expressed as pSTAT5 (MFI) fold change over PBS. ***p value<0.001, one-way Brown-Forsythe and Welch ANOVA test with Dunnett correction for multiple comparison. Each group was compared to the untreated condition.

Data are representative of at least 3 independent experiments.

FIG. 7. Gene expression profile in KCs upon in vivo IL-2c treatment.

(A) Principal component analysis (PCA) visualization of gene expression data from the indicated mice. The percentage of variance explained by PC1 and PC2 is 76% and 10%, respectively. (B) Heatmap of differentially expressed genes (FDR<0.05) upon IL-2c treatment in KCs. 1515 genes were up- and 2558 genes were down-regulated. log₂ TPM values were scaled by row across samples (Z-score) and hierarchical clustering was applied as clustering method.

FIG. 8. Regulated processes in KCs upon in vivo IL-2c treatment.

(A) Clustering of top significant (EnrichR Combined Score>100, FDR<0.05) Gene Ontology Biological Processes and Kyoto Encyclopedia of Genes and Genomes (KEGG) pathways of biological processes up-regulated in KCs upon in vivo IL-2c treatment. The thermal scale represents the Jaccard Similarity Coefficient between every gene set pair (blue representing a 0 Similarity Coefficient, red a 1 Similarity Coefficient). (B) Clustering of top significant (EnrichR Combined Score>30, FDR<0.05) Gene Ontology Biological Processes and KEGG pathways of biological processes down-regulated in KCs upon in vivo IL-2c treatment. The thermal scale represents the Jaccard Similarity Coefficient between every gene set pair (blue representing a 0 Similarity Coefficient, red a 1 Similarity Coefficient).

FIG. 9: Genes associated to cross-presentation are upregulated in KC's upon in vivo II-2c treatment

(A-E) Schematic representation (A) and expression heatmap (B-E) of selected genes belonging to biological processes implicated in antigen cross-presentation upregulated in KCs after IL-2c treatment. Values are in Z-score, calculated from scaling by row the Log₂ (TPM) values. (F) Cytoscape network of top significant (EnrichR Combined Score>100, FDR<0.05) Gene Ontology Biological Processes and KEGG pathways of up-regulated processes. Red dots indicate enriched terms, green dots indicate the relative genes found enriched.

FIG. 10. KC enrichment upon immunomagnetic separation.

(A) Representative flow cytometry plots of KCs (F4/80⁺ cells) in liver non parenchymal cells (LNPCs) before (left panel) and after (right panel) positive immunomagnetic separation. Numbers represent the percentage of cells within the indicated gate. (B) Representative flow cytometry plots of DCs (CD11c⁺ MHC-II^{high}) in LNPCs before (left panel) and after (right panel) immunomagnetic separation.

Data are representative of at least 3 independent experiments.

FIG. 11. Numbers and MHC-I expression in KCs from TAP1^{-/-} mice.

(A-B) Representative histograms (A) and MFI (B) of H2-Kb expression on KCs (identified as live, CD45⁺, F4/80⁺, TIM4⁺ cells) isolated from C57BL/6 (WT, blue line, n=3) or TAP1^{-/-} (red line, n=4) mice. * p value<0.01, two tailed Mann-Whitney U-test. (C) Percentage of KCs among CD45⁺ LNPCs in the indicated mice.

Data are representative of at least 3 independent experiments.

FIG. 12. Gene expression profile of KC1 and KC2.

Heatmap of differentially expressed genes (FDR<0.05) between KC1 and KC2. 3424 genes were hyper- and 4153 genes were hypo-expressed in KC2 compared to KC1. Values are in Z-score, calculated from scaling by row the Log₂(TPM) values and hierarchical clustering was applied as clustering method.

FIG. 13. IL-2c treatment alone or liver inflammation have no impact on KC1/KC2 ratio.

(A) Schematic representation of the experimental setup. HBV Tg mice were treated with PBS (n=4) or IL-2c (n=3) and livers were collected and analyzed four days after treatment. (B) Levels of ALT in the serum of the indicated mice at the indicated timepoints. (C) Numbers of KCs (identified as live, CD45⁺, F4/80⁺, TIM4⁺ cells) per gram of liver in the indicated mice. (D) Representative flow cytometry plots of KC1 (CD206⁻ ESAM⁻) and KC2 (CD206⁺ ESAM⁺) in the indicated mice. Numbers indicate percentages within the indicated gate. (E) KC1/KC2 ratio in the indicated mice. (F) Schematic representation of the experimental setup. MUP-core mice were injected with PBS or in vitro-differentiated effector Cor93 T cells (Cor93 TE, n=3). Livers were collected and analyzed one day after T cell transfer. (G) Levels of ALT in the serum of indicated mice at the indicated timepoints. (H) Numbers of KCs per gram of liver in the indicated mice. ***p value<0.001, two-way ANOVA with Sidak's multiple comparison test. (I) Representative flow cytometry plots of KC1 (CD206⁻ ESAM⁻) and KC2 (CD206⁺ ESAM⁺) in the indicated mice. (J) KC1/KC2 ratio in the indicated mice. Numbers indicate percentages within the indicated gate.

Data are representative of at least 3 independent experiments.

FIG. 14. LSECs and KC2, but not KC1, express Chd5.

(A) Schematic representation of the experimental setup. Cdh5^{CreERT2}; Rosa26^{tdTomato} mice were treated with tamoxifen and livers were collected and analyzed 7 days after treatment (n=3). (B) Gating strategy for KC1, KC2 and LSECs. (C-D) Representative histograms (C) and percentage (D) of tdTomato expression on KC1 (blue) and KC2 (red) and LSECs (green).

Data are representative of at least 3 independent experiments.

FIG. 15: GM-CSF blockade increases KC2.

(A) Numbers of KCs in the liver of the indicated mice. KCs were identified as live, CD45⁺, F4/80⁺, Tim4⁺ cells. (B) KC1/KC2 ratio in the indicated mice. KC1 were identified as CD206⁻ ESAM⁻ KCs; KC2 were identified as CD206⁺ ESAM⁺ KCs.

FIG. 16: KC1 and KC2 markers (murine and human).

(A) Schematic representation of flow cytometry plot of KC1/KC2/LSEC gating strategy. KC1 are defined as ESAM⁻ CD206⁻ KCs. KC2 are defined as ESAM⁺ CD206⁺ KCs. (B) List of cell markers for human and murine KC1 and KC2 cells.

FIG. 17: NK cells depletion improves CD8⁺ T cell activity.

A) Schematic representation of the experimental set up. HBV-Tg mice were injected with the a-NK1.1 depleting antibody (a-NK1.1, n=8, red dots) or PBS (n=8, blue dots) prior to receiving 1×10⁶ HBcAg-specific naïve CD8⁺ T cells (Cor93 T_{N}) followed, 24 hours later, by IL-2/anti-IL-2 complexes (IL-2c, empty dots, n=8) or PBS (full dots, n=8). Livers were collected and analysed 5 days after T cell transfer. B) Absolute number of Group 1 ILCs (NK cells and ILC1s) obtained from the liver of the indicated mice is shown. C-D) Absolute number of (C) Cor93 T_{N} and of (D) IFN-γ producing Cor93 T_{N} in the liver of the indicated mice. E) Serum transaminase activity (ALT, U/L) in HBV Tg mice after Cor93 T_{N} injection. * p-value<0.05, ** p value<0.01,*** p-value<0.001, Two-way Anova.

FIG. 18: Effect of OX40-OX40L axis perturbation on naive HBV-specific CD8 T⁺ cells undergoing intrahepatic priming.

(A) Schematic representation of the experimental setup. 10⁶ Cor93 T naive (Cor93 T_{N}) were transferred to MUP-core recipients and recovered from the liver after 5 days. Where indicated, mice were injected with anti-OX40 agonist antibody or with anti-OX40L blocking antibody immediately after Cor93 T^{N} cell transfer and every other day (Publicover J. et al., Sci TranslMed. 2018). (B) Soluble ALT levels detected in the sera of indicated mice. (C) Absolute number of intrahepatic Cor93 T cells recovered from the livers of indicated mice at day 5 after adoptive cell transfer. (D) Quantification of intrahepatic INFγ-producing Cor93 T cells stimulated ex vivo with cognate cor₉₃₋₁₀₀ peptide. (E) Confocal immunofluorescence and (F) H&E micrograph of liver sections from indicated mice five days after Cor93 T_{N} cell transfer (scale bar 150 μm and 100 μm respectively).

### DETAILED DESCRIPTION OF THE INVENTION

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

In one aspect, the invention provides an agent that increases the number of Kupffer cells in a subject, or a nucleotide sequence encoding therefor, for use in a method of therapy by increasing liver immune response. In another aspect the invention provides an agent that increases the proportion of Type 2 Kupffer cells (KC2) in relation to Type 1 Kupffer cells (KC1) in a subject, or a nucleotide sequence encoding therefor, for use in a method of therapy by increasing liver immune response.

In another aspect, the invention provides an interleukin that binds to IL-2 receptor (IL-2R), or a nucleotide sequence encoding therefor, for use in a method of therapy by increasing liver immune response, wherein the interleukin is administered simultaneously, sequentially, or separately in combination with an agent that increases the number of Kupffer cells in a subject, or a nucleotide sequence encoding therefor. In another aspect, the invention provides an interleukin that binds to IL-2 receptor (IL-2R), or a nucleotide sequence encoding therefor, for use in a method of therapy by increasing liver immune response, wherein the interleukin is administered simultaneously, sequentially, or separately in combination with an agent that increases the proportion of Type 2 Kupffer cells (KC2) in relation to Type 1 Kupffer cells (KC1) in a subject, or a nucleotide sequence encoding therefor.

The increase in the number of Kupffer cells (e.g. increase in the number of Type 2 Kupffer cells) may be, for example, an increase of at least 10%, 20%, 30%, 40%, 50%, 60%, 70%, 80%, 90%, 100%, 150%, 200%, 250% or 500% following administration of the agent compared to the number of Kupffer cells in an untreated subject under substantially identical conditions.

The increase in the proportion of Type 2 Kupffer cells (KC2) in relation to Type 1 Kupffer cells (KC1) may be, for example, an increase of at least 10%, 20%, 30%, 40%, 50%, 60%, 70%, 80%, 90%, 100%, 150%, 200%, 250% or 500%, preferably at least 50%, of the relative proportion of KC2 following administration of the agent compared to the proportion in an untreated subject under substantially identical conditions.

The agent of the invention may increase the number of Type 2 Kupffer cells (KC2) while the number of Type 1 Kupffer cells (KC1) remains substantially constant. In some embodiments, in an increase in the proportion of Type 2 Kupffer cells (KC2) in relation to Type 1 Kupffer cells (KC1), the number of Type 1 Kupffer cells remain substantially constant. In some embodiments, in an increase in the proportion of Type 2 Kupffer cells (KC2) in relation to Type 1 Kupffer cells (KC1), the number of Type 1 Kupffer cells increases (and the number of Type 2 Kupffer cells increases by a greater amount than the increase in the number of Type 1 Kupffer cells).

Kupffer cells, Type 2 Kupffer cells and Type 1 Kupffer cells may be readily identified and quantified using methods known in the art. For example, flow cytometry (e.g. as disclosed herein) using suitable cell markers may enable identification and quantification of the number of cells in particular cell populations, such as those isolated from a subject or animal model.

In another aspect, the invention provides a granulocyte-macrophage colony-stimulating factor (GM-CSF) inhibitor, or a nucleotide sequence encoding therefor, for use in a method of therapy by increasing liver immune response.

The term “increasing liver immune response” as used herein may refer to increasing T cell immunity in the liver. In general, the liver is understood to be biased towards inducing a state of T cell unresponsiveness or dysfunction, in particular unresponsiveness towards antigens specifically expressed in hepatocytes (for example, leading to a propensity of some hepatotropic viruses, such as HBV, to establish persistent infections). In some embodiments, increasing liver immune response is increasing T cell effector responses, preferably CD8+ T cells (e.g. in dysfunctional CD8+ T cells), such as against antigens specifically expressed in hepatocytes. In some embodiments, the effector responses are against hepatotropic viruses, such as hepatitis virus, preferably HBV. In some embodiments, increasing liver immune response is increasing T cell antiviral activity, preferably CD8+ T cell antiviral activity. In some embodiments, increasing liver immune response is increasing CD8+ T cell effector differentiation in the liver

The increasing liver immune response may, for example, improve methods of treatment, such as treatment or prevention of a liver infection, primary liver tumour or secondary liver tumour.

### Liver Infections

In some embodiments, the liver infection is a virus infection.

In some embodiments, the liver infection is a hepatitis virus infection. In some embodiments, the liver infection is a chronic hepatitis virus infection.

In some embodiments, the liver infection is a hepatitis B virus (HBV) infection. In some embodiments, the liver infection is a hepatitis C virus (HCV) infection.

In some embodiments, the liver infection is a Plasmodium infection, for example a Plasmodium falciparum, Plasmodium vivax, Plasmodium ovale, Plasmodium malariae or Plasmodium knowlesi infection. In some embodiments, the method of therapy is treatment or prevention of malaria.

### Hepatitis

The invention relates to agents for use in the treatment and prevention of liver infections, in particular viral liver infections, such as hepatitis infections.

Hepatitis infections, such as hepatitis B virus (HBV) infection, remain a major public health issue worldwide. For example, it has been estimated that about 248 million individuals were positive for hepatitis B surface antigen, a marker of chronic HBV infection, globally in 2010.

HBV is a non-cytopathic virus that replicates exclusively in hepatocytes without inducing innate immune activation.

Chronic HBV infection is typically acquired at birth or in early childhood, and is particularly prevalent in Asian and African countries where HBV is endemic. The risk of developing chronic infection after exposure drops from ca. 90% in neonates to 1-5% in healthy adults. However, 25% of people who acquire HBV as children will develop primary liver cancer or cirrhosis as adults.

Chronic infections acquired perinatally or in early childhood go through several prolonged and progressive disease phases, including an initial “immune tolerant” phase (characterised by high viremia, normal ALT values and no liver inflammation) that is often followed by an “immune active” phase (in which viremia is lower, ALT values are higher and liver inflammation is present) (Kennedy, P. et al. (2017) Viruses 9: 96; and EASL (2017) Journal of Hepatology 67: 370-398). HBV-specific CD8+ T cells in young immune tolerant chronic HBV patients are considered akin to Ag-specific exhausted T cells that characterise the immune active phase (Fisicaro, P. et al. (2017) Nature Medicine 23: 327-336), as well as to other infection- or cancer-related conditions of immune dysfunction.

HBV and HCV infections can both give rise to hepatocellular carcinomas.

### Kupffer Cells

Kupffer cells (KCs; also known as stellate macrophages and Kupffer-Browicz cells), are highly abundant, intravascular, liver-resident macrophages long known for their scavenger and phagocytic functions. KCs express the complement receptor of the immunoglobulin family CRIg, a critical component of the innate immune system involved in complement clearance of pathogens. KCs are localised in the hepatic sinusoid and can phagocytize pathogens entering from the portal or arterial circulation. KCs also act against particulates and immunoreactive material from the gastrointestinal tract via the portal circulation. KCs are also able to present antigens to CD8⁺ T cells and promote either T cell tolerance or full effector differentiation.

Kupffer cells may express one or more of the markers CD45, F4/80 and/or TIM4. In some embodiments, Kupffer cells are CD45⁺ F4/80⁺ TIM4⁺.

Subsets of Kupffer cells are disclosed herein, which may be referred to herein as Type 1 Kupffer cells (KC1) and Type 2 Kupffer cells (KC2).

Type 1 Kupffer cells (KC1) may lack expression of one or more of the markers ESAM and/or CD206. In some embodiments, Type 1 Kupffer cells (KC1) are ESAM⁻ CD206⁻.

Type 2 Kupffer cells (KC2) may express one or more of the markers ESAM and/or CD206. In some embodiments, Type 2 Kupffer cells (KC2) are ESAM⁺ CD206⁺.

Further cell markers for Type 1 (KC1) and Type 2 (KC2) Kupffer cells are shown in FIG. 16.

Human Type 1 Kupffer cells (KC1) may express one or more of the markers Clec12a, Cd300e, Cd52, S100A8 and/or S100A9. In some embodiments, human Type 1 Kupffer cells (KC1) are Clec12a⁺ Cd300e⁺ Cd52⁺ S100A8⁺ S100A9⁺.

Human Type 2 Kupffer cells (KC2) may express one or more of the markers Slc40a1, Fabp5, Mrc1, Folr2, Lyve1, Vsig4, Cd84, Mertk, Cd72 and/or Cd81. In some embodiments, human Type 2 Kupffer cells (KC2) are Slc40a1⁺ Fabp5⁺ Mrc1⁺ Folr2⁺ Lyve1⁺ Vsig4⁺ Cd84⁺ Mertk⁺ Cd72⁺ Cd81⁺.

As disclosed herein, Type 2 Kupffer cells may be enriched for IL-2 signalling components, particularly the three subunits of the IL-2 receptor (IL-2R), namely CD25, CD122 and CD132. Type 2 Kupffer cells may express one or more of the markers CD25, CD122 and/or CD132. In some embodiments, Type 2 Kupffer cells are CD25⁺ CD122⁺ CD132⁺.

In another aspect, the invention provides an isolated Type 2 Kupffer cell. In another aspect, the invention provides an isolated population of Type 2 Kupffer cells.

In another aspect, the invention provides an isolated Type 1 Kupffer cell. In another aspect, the invention provides an isolated population of Type 1 Kupffer cells.

### GM-CSF

In preferred embodiments, the agent of the invention that increases the number of Kupffer cells in a subject (preferably increases the number of Type 2 Kupffer cells (KC2) in a subject) is a granulocyte-macrophage colony-stimulating factor (GM-CSF) inhibitor.

In preferred embodiments, the agent of the invention that increases the proportion of Type 2 Kupffer cells (KC2) in relation to Type 1 Kupffer cells (KC1) in a subject is a granulocyte-macrophage colony-stimulating factor (GM-CSF) inhibitor.

The cytokine Granulocyte-Macrophage Colony-Stimulating Factor (GM-CSF) is produced by many cells such as macrophages, T cells, mast cells, natural killer cells, endothelial cells and fibroblasts and is generally perceived as a pro-inflammatory cytokine. It is also known to regulate macrophage differentiation.

GM-CSF is a monomeric glycoprotein that stimulates stem cells to produce granulocytes (neutrophils, eosinophils and basophils) and monocytes. GM-CSF can also enhance neutrophil migration and alter receptors that are expressed on the surface of mature cells of the immune system.

GM-CSF signals are mediated by the GM-CSF receptor (GM-CSF-R) consisting of specific ligand-binding alpha-chain (GM-CSF-Ra) and signal-transducing beta-chain (GM-CSF-Rb).

In some embodiments, the GM-CSF inhibitor decreases the activity of GM-CSF or GM-CSF Receptor (GM-CSF-R), preferably GM-CSF.

In some embodiments, the activity may decreased by at least 10%, at least 20%, at least 30%, at least 40%, at least 50%, at least 60%, at least 70%, at least 80%, at least 90%, at least 95% or 100% when compared to the activity in the absence of the inhibitor.

Methods for determining GM-CSF activity are well known in the art. For example, a reporter cell line (such as iLite® GM-CSF Assay Cells) may be used that is responsive to GM-CSF through expression of the reporter (e.g. Firefly Luciferase). Normalisation of cell counts and other effects may be achieved using a second reporter under the control of a constitutive promotor.

In some embodiments, the GM-CSF inhibitor is an antibody or a fragment thereof that binds GM-CSF or GM-CSF Receptor (GM-CSF-R), preferably GM-CSF. In some embodiments, the antibody or fragment thereof depletes GM-CSF or GM-CSF-R.

In some embodiments, the GM-CSF inhibitor is a GM-CSF Receptor (GM-CSF-R) antagonist.

The term “antibody” refers to complete antibodies or antibody fragments capable of binding to a selected target, and including Fv, ScFv, F(ab′) and F(ab′)2, monoclonal and polyclonal antibodies, engineered antibodies including chimeric, CDR-grafted and humanised antibodies, and artificially selected antibodies produced using phage display or alternative techniques.

Antibodies that specifically bind a target antigen can be prepared using methods well known in the art. Such methods include phage display, methods to generate human or humanised antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

In addition, alternatives to classical antibodies may also be used in the invention, for example “avibodies”, “avimers”, “anticalins”, “nanobodies” and “DARPins”.

Suitable anti-GM-CSF antibodies are disclosed in Bonaventura, A. et al. (2020) Front. Immunol. 11: 1625.

In some embodiments, the GM-CSF inhibitor is selected from the group consisting of Gimsilumab, Otilimab, Namilumab and Lenzilumab.

For example, Otilimab is a fully human, monoclonal antibody that specifically binds to and neutralises GM-CSF. Many such antibodies have been studied in the treatment of RA.

Inhibitors of GM-CSF also include antibodies that bind GM-CSF-R so that it cannot interact with GM-CSF.

In some embodiments, the GM-CSF inhibitor is Mavtrilimumab or CSL311.

Mavtrilimumab, a high affinity, monoclonal IgG4 antibody against GM-CSF-Ra, originally studied in RA for safety and efficacy and later investigated in COVID-19 patients. CSL311 is an example of a monoclonal antibody targeting GM-CSF-Rb.

In some embodiments, the GM-CSF inhibitor reduces expression of GM-CSF or GM-CSF Receptor (GM-CSF-R), preferably GM-CSF.

Measurement of the level or amount of a gene product may be carried out by any suitable method, for example including comparison of mRNA transcript levels, protein or peptide levels, between a treated cell and comparable cell which has not been treated according to the present invention.

The term “treated cell” as used herein may refer to a cell that has been modified according to the present invention, e.g. to modulate the expression or activity of GM-CSF and/or GM-CSF-R protein, or to modify the nucleic acid sequence of at least one gene encoding GM-CSF and/or GM-CSF-R.

The expression of specific genes encoding GM-CSF and/or GM-CSF-R can be measured by measuring transcription and/or translation of the gene. Methods for measuring transcription are well known in the art and include, for example, northern blot, RNA-Seq, in situ hybridization, DNA microarrays and RT-PCR. Alternatively, the expression of a gene may be measured by measuring the level of the gene product, for example the protein encoded by said gene.

In some embodiments, the expression may decreased by at least 10%, at least 20%, at least 30%, at least 40%, at least 50%, at least 60%, at least 70%, at least 80%, at least 90%, at least 95% or 100% when compared to the expression in the absence of the inhibitor.

By way of example, the GM-CSF and/or GM-CSF-R inhibitor may be a small molecule inhibitor or a regulatory RNA. Regulatory RNAs are non-coding RNA molecules that play a role in cellular processes such as activation or inhibition processes. The regulatory RNAs may be a small inhibitory RNA (siRNA), a small hairpin RNA (shRNA), a micro RNA (miRNA) and/or their precursors, an antisense nucleic acid. Other regulatory RNAs are described in Morris, K. V. and Mattick, J. S., 2014. Nature Reviews Genetics, 15(6), pp. 423-437.

Inhibition (e.g. of the GM-CSF and/or GM-CSF-R) may be achieved using post-transcriptional gene silencing (PTGS). Post-transcriptional gene silencing mediated by double-stranded RNA (dsRNA) is a conserved cellular defence mechanism for controlling the expression of foreign genes. It is thought that the random integration of elements such as transposons or viruses causes the expression of dsRNA which activates sequence-specific degradation of homologous single-stranded mRNA or viral genomic RNA. The silencing effect is known as RNA interference (RNAi) (Ralph et al. (2005) Nat. Medicine 11: 429-433). The mechanism of RNAi involves the processing of long dsRNAs into duplexes of about 21-25 nucleotide (nt) RNAs. These products are called small interfering or silencing RNAs (siRNAs) which are the sequence-specific mediators of mRNA degradation. In differentiated mammalian cells, dsRNA>30 bp has been found to activate the interferon response leading to shut-down of protein synthesis and non-specific mRNA degradation (Stark et al. (1998) Ann. Rev. Biochem. 67: 227-64). However, this response can be bypassed by using 21 nt siRNA duplexes (Elbashir et al. (2001) EMBO J. 20: 6877-88; Hutvagner et al. (2001) Science 293: 834-8) allowing gene function to be analysed in cultured mammalian cells.

shRNAs consist of short inverted RNA repeats separated by a small loop sequence. These are rapidly processed by the cellular machinery into 19-22 nt siRNAs, thereby suppressing the target gene expression.

Micro-RNAs (miRNAs) are small (22-25 nucleotides in length) noncoding RNAs that can effectively reduce the translation of target mRNAs by binding to their 3′ untranslated region (UTR). Micro-RNAs are a very large group of small RNAs produced naturally in organisms, at least some of which regulate the expression of target genes. Founding members of the micro-RNA family are let-7 and lin-4. The let-7 gene encodes a small, highly conserved RNA species that regulates the expression of endogenous protein-coding genes during worm development. The active RNA species is transcribed initially as an ˜70 nt precursor, which is post-transcriptionally processed into a mature ˜21 nt form. Both let-7 and lin-4 are transcribed as hairpin RNA precursors which are processed to their mature forms by Dicer enzyme.

The antisense concept is to selectively bind short, possibly modified, DNA or RNA molecules to messenger RNA in cells and prevent the synthesis of the encoded protein.

Methods for the design of siRNAs, shRNAs, miRNAs and antisense DNAs/RNAs to modulate the expression of a target protein, and methods for the delivery of these agents to a cell of interest are well known in the art.

### Interleukin

Interleukins (ILs) are a group of cytokines, the majority of which are made by helper CD4 T cells, as well as monocytes, macrophages and endothelial cells. They function in promoting the development and differentiation of T and B lymphocytes, and hematopoietic cells.

In some embodiments, the interleukin is selected from the group consisting of IL-2, IL-7 or IL-15, preferably the interleukin is IL-2.

### Interleukin-2 (IL-2)

Interleukin-2 (IL-2) plays a role in the regulation of the activities of white blood cells that are responsible for immunity. IL-2 is part of the natural response to microbial infection, and is involved in the discrimination between “self” and “non-self”. IL-2 mediates its effects by binding to IL-2 receptors, which are expressed by lymphocytes. Sources of IL-2 include activated CD4+ T cells, activated CD8+ T cells, NK cells, dendritic cells and macrophages.

In preferred embodiments, the IL-2 is human IL-2.

An example IL-2 sequence is:

(SEQ ID NO: 1)
MYRMQLLSCIALSLALVTNSAPTSSSTKKTQLQLEHLLLDLQMILNGI
NNYKNPKLTRMLTFKFYMPKKATELKHLQCLEEELKPLEEVLNLAQSK
NFHLRPRDLISNINVIVLELKGSETTFMCEYADETATIVEFLNRWITF
CQSIISTLT

An example nucleotide sequence encoding IL-2 is:

(SEQ ID NO: 2)
ATGTACAGGATGCAACTCCTGTCTTGCATTGCACTAAGTCTTGCACTT
GTCACAAACAGTGCACCTACTTCAAGTTCTACAAAGAAAACACAGCTA
CAACTGGAGCATTTACTGCTGGATTTACAGATGATTTTGAATGGAATT
AATAATTACAAGAATCCCAAACTCACCAGGATGCTCACATTTAAGTTT
TACATGCCCAAGAAGGCCACAGAACTGAAACATCTTCAGTGTCTAGAA
GAAGAACTCAAACCTCTGGAGGAAGTGCTAAATTTAGCTCAAAGCAAA
AACTTTCACTTAAGACCCAGGGACTTAATCAGCAATATCAACGTAATA
GTTCTGGAACTAAAGGGATCTGAAACAACATTCATGTGTGAATATGCT
GATGAGACAGCAACCATTGTAGAATTTCTGAACAGATGGATTACCTTT
TGTCAAAGCATCATCTCAACACTGACTTGA

In some embodiments, the IL-2 is encoded by a nucleotide sequence that has at least 70%, 80%, 90%, 95%, 96%, 97%, 98%, 99% or 100% identity to SEQ ID NO: 2, preferably wherein the protein encoded by the nucleotide sequence substantially retains the natural function of the protein represented by SEQ ID NO: 1.

In some embodiments, the IL-2 is encoded by a nucleotide sequence that encodes an amino acid sequence that has at least 70%, 80%, 90%, 95%, 96%, 97%, 98%, 99% or 100% identity to SEQ ID NO: 1, preferably wherein the amino acid sequence substantially retains the natural function of the protein represented by SEQ ID NO: 1.

In some embodiments, the IL-2 comprises or consists of an amino acid sequence that has at least 70%, 80%, 90%, 95%, 96%, 97%, 98%, 99% or 100% identity to SEQ ID NO: 1, preferably wherein the amino acid sequence substantially retains the natural function of the protein represented by SEQ ID NO: 1.

### Interleukin-7 (IL-7)

Interleukin-7 (IL-7) is a hematopoietic growth factor that may be secreted by stromal cells in the bone marrow and thymus. IL-7 may also be produced by keratinocytes, dendritic cells, hepatocytes, neurons and epithelial cells, but is typically not produced by normal lymphocytes.

In preferred embodiments, the IL-7 is human IL-7.

An example IL-7 sequence is:

(SEQ ID NO: 3)
MFHVSFRYIFGLPPLILVLLPVASSDCDIEGKDGKQYESVLMVSIDQL
LDSMKEIGSNCLNNEFNFFKRHICDANKEGMFLFRAARKLRQFLKMNS
TGDFDLHLLKVSEGTTILLNCTGQVKGRKPAALGEAQPTKSLEENKSL
KEQKKLNDLCFLKRLLQEIKTCWNKILMGTKEH

An example nucleotide sequence encoding IL-7 is:

(SEQ ID NO: 4)
ATGTTCCATGTTTCTTTTAGGTATATCTTTGGACTTCCTCCCCTGATC
CTTGTTCTGTTGCCAGTAGCATCATCTGATTGTGATATTGAAGGTAAA
GATGGCAAACAATATGAGAGTGTTCTAATGGTCAGCATCGATCAATTA
TTGGACAGCATGAAAGAAATTGGTAGCAATTGCCTGAATAATGAATTT
AACTTTTTTAAAAGACATATCTGTGATGCTAATAAGGAAGGTATGTTT
TTATTCCGTGCTGCTCGCAAGTTGAGGCAATTTCTTAAAATGAATAGC
ACTGGTGATTTTGATCTCCACTTATTAAAAGTTTCAGAAGGCACAACA
ATACTGTTGAACTGCACTGGCCAGGTTAAAGGAAGAAAACCAGCTGCC
CTGGGTGAAGCCCAACCAACAAAGAGTTTGGAAGAAAATAAATCTTTA
AAGGAACAGAAAAAACTGAATGACTTGTGTTTCCTAAAGAGACTATTA
CAAGAGATAAAAACTTGTTGGAATAAAATTTTGATGGGCACTAAAGAA
CACTGA

In some embodiments, the IL-7 is encoded by a nucleotide sequence that has at least 70%, 80%, 90%, 95%, 96%, 97%, 98%, 99% or 100% identity to SEQ ID NO: 4, preferably wherein the protein encoded by the nucleotide sequence substantially retains the natural function of the protein represented by SEQ ID NO: 3.

In some embodiments, the IL-7 is encoded by a nucleotide sequence that encodes an amino acid sequence that has at least 70%, 80%, 90%, 95%, 96%, 97%, 98%, 99% or 100% identity to SEQ ID NO: 3, preferably wherein the amino acid sequence substantially retains the natural function of the protein represented by SEQ ID NO: 3.

In some embodiments, the IL-7 comprises or consists of an amino acid sequence that has at least 70%, 80%, 90%, 95%, 96%, 97%, 98%, 99% or 100% identity to SEQ ID NO: 3, preferably wherein the amino acid sequence substantially retains the natural function of the protein represented by SEQ ID NO: 3.

### Interleukin-15 (IL-15)

Interleukin-15 (IL-15) has structural similarity to IL-2. IL-15 is secreted by mononuclear phagocytes following viral infection. It induces proliferation of natural killer cells.

In preferred embodiments, the IL-15 is human IL-15.

An example IL-15 sequence is:

(SEQ ID NO: 5)
MRISKPHLRSISIQCYLCLLLNSHFLTEAGIHVFILGCFSAGLPKTEA
NWVNVISDLKKIEDLIQSMHIDATLYTESDVHPSCKVTAMKCFLLELQ
VISLESGDASIHDTVENLIILANNSLSSNGNVTESGCKECEELEEKNI
KEFLQSFVHIVQMFINTS

An example nucleotide sequence encoding IL-15 is:

(SEQ ID NO: 6)
ATGAGAATTTCGAAACCACATTTGAGAAGTATTTCCATCCAGTGCTAC
TTGTGTTTACTTCTAAACAGTCATTTTCTAACTGAAGCTGGCATTCAT
GTCTTCATTTTGGGCTGTTTCAGTGCAGGGCTTCCTAAAACAGAAGCC
AACTGGGTGAATGTAATAAGTGATTTGAAAAAAATTGAAGATCTTATT
CAATCTATGCATATTGATGCTACTTTATATACGGAAAGTGATGTTCAC
CCCAGTTGCAAAGTAACAGCAATGAAGTGCTTTCTCTTGGAGTTACAA
GTTATTTCACTTGAGTCCGGAGATGCAAGTATTCATGATACAGTAGAA
AATCTGATCATCCTAGCAAACAACAGTTTGTCTTCTAATGGGAATGTA
ACAGAATCTGGATGCAAAGAATGTGAGGAACTGGAGGAAAAAAATATT
AAAGAATTTTTGCAGAGTTTTGTACATATTGTCCAAATGTTCATCAAC
ACTTCTTGA

In some embodiments, the IL-15 is encoded by a nucleotide sequence that has at least 70%, 80%, 90%, 95%, 96%, 97%, 98%, 99% or 100% identity to SEQ ID NO: 6, preferably wherein the protein encoded by the nucleotide sequence substantially retains the natural function of the protein represented by SEQ ID NO: 5.

In some embodiments, the IL-15 is encoded by a nucleotide sequence that encodes an amino acid sequence that has at least 70%, 80%, 90%, 95%, 96%, 97%, 98%, 99% or 100% identity to SEQ ID NO: 5, preferably wherein the amino acid sequence substantially retains the natural function of the protein represented by SEQ ID NO: 5.

In some embodiments, the IL-15 comprises or consists of an amino acid sequence that has at least 70%, 80%, 90%, 95%, 96%, 97%, 98%, 99% or 100% identity to SEQ ID NO: 5, preferably wherein the amino acid sequence substantially retains the natural function of the protein represented by SEQ ID NO: 5.

### Interleukin-2 Receptor

The IL-2 receptor consists of a heterocomplex of up to three subunits: a (CD25), b (CD122) and the common g chain (CD132). Although each receptor subunit can independently bind IL-2 with a low affinity (K_{d}: ˜10⁻⁸-10⁻⁷ M), only the intermediate-affinity bg dimeric (K_{d}: ˜10⁻⁹ M) and the high-affinity abg trimeric (K_{d}: ˜10⁻¹¹ M) receptors mediate intracellular signal transduction. In addition to T cells and NK cells, myeloid cells have been reported to express the intermediate-affinity bg receptor, with some DC subtypes displaying the three subunits of the IL-2 receptor.

### NK Cell Depletion

In another aspect, the invention provides an agent that depletes NK cells in a subject, or a nucleotide sequence encoding therefor, for use in a method of therapy by increasing liver immune response.

In some embodiments, the method of therapy is treatment or prevention of a liver infection. In some embodiments, the method of therapy is treatment or prevention of a primary liver tumour. In some embodiments, the method of therapy is treatment or prevention of a secondary liver tumour.

In another aspect, the invention provides an agent that depletes NK cells in a subject, or a nucleotide sequence encoding therefor, for use in treatment or prevention of a liver infection. In another aspect, the invention provides an agent that depletes NK cells in a subject, or a nucleotide sequence encoding therefor, for use in treatment or prevention of a primary or secondary liver tumour.

In some embodiments, the agent that depletes NK cells is administered simultaneously, sequentially or separately in combination with (a) an agent that increases the number of Kupffer cells in a subject, or a nucleotide sequence encoding therefor; and/or (b) an interleukin that binds to IL-2 receptor (IL-2R), or a nucleotide sequence encoding therefor.

In some embodiments, the agent that depletes NK cells is administered simultaneously, sequentially or separately in combination with (a) an agent that increases the proportion of Type 2 Kupffer cells (KC2) in relation to Type 1 Kupffer cells (KC1) in a subject, or a nucleotide sequence encoding therefor; and/or (b) an interleukin that binds to IL-2 receptor (IL-2R), or a nucleotide sequence encoding therefor.

In some embodiments, the agent that depletes NK cells is an anti-NK1.1 antibody. NK1.1 is also known as CD161b/CD161c, KLRB1, NKR-P1A and Ly-55. Example anti-NK1.1 antibodies are known in the art and include clone PK136 (Bioxcell).

In some embodiments, the agent that depletes NK cells is administered simultaneously, sequentially or separately in combination with an agent that inhibits OX40.

### OX40 Inhibition

In another aspect, the invention provides an agent that inhibits OX40, or a nucleotide sequence encoding therefor, for use in a method of therapy by increasing liver immune response.

In some embodiments, the method of therapy is treatment or prevention of a liver infection. In some embodiments, the method of therapy is treatment or prevention of a primary liver tumour. In some embodiments, the method of therapy is treatment or prevention of a secondary liver tumour.

In another aspect, the invention provides an agent that inhibits OX40, or a nucleotide sequence encoding therefor, for use in treatment or prevention of a liver infection. In another aspect, the invention provides an agent that inhibits OX40, or a nucleotide sequence encoding therefor, for use in treatment or prevention of a primary or secondary liver tumour.

In some embodiments, the agent that inhibits OX40 is administered simultaneously, sequentially or separately in combination with (a) an agent that increases the number of Kupffer cells in a subject, or a nucleotide sequence encoding therefor; and/or (b) an interleukin that binds to IL-2 receptor (IL-2R), or a nucleotide sequence encoding therefor.

In some embodiments, the agent that inhibits OX40 is administered simultaneously, sequentially or separately in combination with (a) an agent that increases the proportion of Type 2 Kupffer cells (KC2) in relation to Type 1 Kupffer cells (KC1) in a subject, or a nucleotide sequence encoding therefor; and/or (b) an interleukin that binds to IL-2 receptor (IL-2R), or a nucleotide sequence encoding therefor.

In some embodiments, the agent that inhibits OX40 is an OX40 agonist. The OX40 agonist may be an anti-OX40 antibody. Example anti-OX40 antibodies are known in the art and include clone 0X-86 (BioXcell).

In some embodiments, the agent that inhibits OX40 is an OX40L antagonist. The OX40L antagonist may be an anti-OX40L antibody. Example anti-OX40L antibodies are known in the art and include clone RM134L (BioXcell).

In some embodiments, the agent that inhibits OX40 is administered simultaneously, sequentially or separately in combination with an agent that depletes NK cells.

### Expression Control Sequences

The nucleotide sequence and vector of the invention may include elements allowing for the expression of the nucleotide sequence(s) encoding the agent and/or interleukin. These may be referred to as expression control sequences. Thus, the nucleotide sequence and vector may comprise one or more expression control sequences (e.g. comprising a promoter sequence) operably linked to the nucleotide sequence(s) encoding the agent and/or interleukin.

By “operably linked”, it is to be understood that the individual components are linked together in a manner which enables them to carry out their function substantially unhindered (e.g. a promoter may be operably linked to a nucleotide of interest to promote expression of the nucleotide of interest in a cell).

### Promoters and Enhancers

Any suitable promoter and/or enhancer may be used, the selection of which may be readily made by the skilled person. The promoter sequence may be constitutively active (i.e. operational in any host cell background), or alternatively may be active only in a specific host cell environment, thus allowing for targeted expression of the nucleotide of interest (e.g. the agent and/or interleukin) in a particular cell type (e.g. a tissue-specific promoter). The promoter may show inducible expression in response to presence of another factor, for example a factor present in a host cell. In any event, where the nucleotide sequence or vector is administered for therapy, it is preferred that the promoter should be functional in the target cell background.

Preferably, the expression control sequences enable liver-specific expression of the agent and/or interleukin, for example confined only to liver cells, such as hepatocytes. Examples of liver-specific promoters include the hepatocyte-specific promoters, liver sinusoidal endothelial cell-specific promoters and Kupffer cell-specific promoters disclosed herein (e.g. selected from the group consisting of an ET promoter, albumin promoter, transthyretin promoter, alpha1-antitrypsin promoter, apoE/alpha1-antitrypsin promoter, vascular endothelial cadherin (VEC) promoter, intercellular adhesion molecule 2 (ICAM2) promoter, foetal liver kinase 1 (Flk1) promoter, Tie2 promoter and CD11b promoter).

In some embodiments, the nucleotide sequence or vector comprises a hepatocyte-specific promoter and/or enhancer operably linked to the nucleotide sequence encoding the agent and/or interleukin.

In some embodiments, the hepatocyte-specific promoter is selected from the group consisting of an ET promoter, albumin promoter, transthyretin promoter, alpha1-antitrypsin promoter and apoE/alpha1-antitrypsin promoter.

The hepatocyte-specific Enhanced Transthyretin (ET) promoter is described in Vigna, E. et al. (2005) Mol. Ther. 11: 763-775, and is composed of synthetic hepatocyte-specific enhancers and transthyretin promoter.

In preferred embodiments, the promoter is an ET promoter.

An example ET promoter sequence is:

(SEQ ID NO: 8)
CGCGAGTTAATAATTACCAGCGCGGGCCAAATAAATAATCCGCGAGGG
GCAGGTGACGTTTGCCCAGCGCGCGCTGGTAATTATTAACCTCGCGAA
TATTGATTCGAGGCCGCGATTGCCGCAATCGCGAGGGGCAGGTGACCT
TTGCCCAGCGCGCGTTCGCCCCGCCCCGGACGGTATCGATAAGCTTAG
GAGCTTGGGCTGCAGGTCGAGGGCACTGGGAGGATGTTGAGTAAGATG
GAAAACTACTGATGACCCTTGCAGAGACAGAGTATTAGGACATGTTTG
AACAGGGGCCGGGCGATCAGCAGGTAGCTCTAGAGGATCCCCGTCTGT
CTGCACATTTCGTAGAGCGAGTGTTCCGATACTCTAATCTCCCTAGGC
AAGGTTCATATTTGTGTAGGTTACTTATTCTCCTTTTGTTGACTAAGT
CAATAATCAGAATCAGCAGGTTTGGAGTCAGCTTGGCAGGGATCAGCA
GCCTGGGTTGGAAGGAGGGGGTATAAAAGCCCCTTCACCAGGAGAAGC
CGTCACACAGATCCACAAGCTCCTG

In some embodiments, the nucleotide sequence or vector comprises a promoter with a nucleotide sequence that has at least 75%, 80%, 85% 90%, 95%, 96%, 97%, 98% or 99% identity to SEQ ID NO: 8 operably linked to the nucleotide sequence encoding the agent and/or interleukin. Preferably, wherein the promoter substantially retains the functional activity of the promoter represented by SEQ ID NO: 8.

In other embodiments, the nucleotide sequence or vector comprises a promoter with the nucleotide sequence of SEQ ID NO: 8 operably linked to the nucleotide sequence encoding the agent and/or interleukin.

The albumin promoter is described in Follenzi, A. et al (2004) Blood 103: 3700-3709.

An example albumin promoter sequence is:

(SEQ ID NO: 9)
GGCATGCTTCCATGCCAAGGCCCACACTGAAATGCTCAAATGGGAGAC
AAAGAGATTAAGCTCTTATGTAAAATTTGCTGTTTTACATAACTTTAA
TGAATGGACAAAGTCTTGTGCATGGGGGTGGGGGTGGGGTTAGAGGGG
AACAGCTCCAGATGGCAAACATACGCAAGGGATTTAGTCAAACAACTT
TTTGGCAAAGATGGTATGATTTTGTAATGGGGTAGGAACCAATGAAAT
GCGAGGTAAGTATGGTTAATGATCTACAGTTATTGGTTAAAGAAGTAT
ATTAGAGCGAGTCTTTCTGCACACAGATCACCTTTCCTATCAACCCC

In some embodiments, the nucleotide sequence or vector comprises a promoter with a nucleotide sequence that has at least 75%, 80%, 85% 90%, 95%, 96%, 97%, 98% or 99% identity to SEQ ID NO: 9 operably linked to the nucleotide sequence encoding the agent or interleukin. Preferably, wherein the promoter substantially retains the functional activity of the promoter represented by SEQ ID NO: 9.

In other embodiments, the nucleotide sequence or vector comprises a promoter with the nucleotide sequence of SEQ ID NO: 9 operably linked to the nucleotide sequence encoding the agent and/or interleukin.

An alpha1-antitrypsin (AAT) promoter is described in Hafenrichter, D. G. et al. (1994) Blood 84: 3394-3404 and W02016146757, and an apoE/alpha1-antitrypsin promoter is described in Miao, C. H. et al. (2000) Mol Ther 1: 522-532 and WO2001098482.

Other suitable promoters, which are not liver specific, include the PGK promoter.

### MicroRNA (miRNA) Target Sequences

The nucleotide sequence or vector of the invention may comprise elements which prevent or reduce the expression of the encoded transgene, for example in certain tissues. Such elements could be recognition sequences which bind or interact with modulators. The modulators could be endogenous modulators present in a cell. Alternatively, the modulators could be exogenous molecules which are introduced into the cell. Preferably, the modulators are microRNAs.

MicroRNA genes are scattered across all human chromosomes, except for the Y chromosome. They can be either located in non-coding regions of the genome or within introns of protein-coding genes. Around 50% of miRNAs appear in clusters which are transcribed as polycistronic primary transcripts. Similar to protein-coding genes, miRNAs are usually transcribed from polymerase-II promoters, generating a so-called primary miRNA transcript (pri-miRNA). This pri-miRNA is then processed through a series of endonucleolytic cleavage steps, performed by two enzymes belonging to the RNAse Type III family, Drosha and Dicer. From the pri-miRNA, a stem loop of about 60 nucleotides in length, called miRNA precursor (pre-miRNA), is excised by a specific nuclear complex, composed of Drosha and DiGeorge syndrome critical region gene (DGCR8), which crops both strands near the base of the primary stem loop and leaves a 5′ phosphate and a 2 bp long, 3′ overhang. The pre-miRNA is then actively transported from the nucleus to the cytoplasm by RAN-GTP and Exportin. Then, Dicer performs a double strand cut at the end of the stem loop not defined by the Drosha cut, generating a 19-24 bp duplex, which is composed of the mature miRNA and the opposite strand of the duplex, called miRNA*. In agreement with the thermodynamic asymmetry rule, only one strand of the duplex is selectively loaded into the RNA-induced silencing complex (RISC), and accumulates as the mature microRNA. This strand is usually the one whose 5′ end is less tightly paired to its complement, as was demonstrated by single-nucleotide mismatches introduced into the 5′ end of each strand of siRNA duplexes. However, there are some miRNAs that support accumulation of both duplex strands to similar extent.

MicroRNAs trigger RNAi, very much like small interfering RNAs (siRNA) which are extensively used for experimental gene knockdown. The main difference between miRNA and siRNA is their biogenesis. Once loaded into RISC, the guide strand of the small RNA molecule interacts with mRNA target sequences preferentially found in the 3′ untranslated region (3′UTR) of protein-coding genes. It has been shown that nucleotides 2-8 counted from the 5′ end of the miRNA, the so-called seed sequence, are essential for triggering RNAi. If the whole guide strand sequence is perfectly complementary to the mRNA target, as is usually the case for siRNAs and plant miRNAs, the mRNA is endonucleolytically cleaved by involvement of the Argonaute (Ago) protein, also called “slicer” of the small RNA duplex into the RNA-induced silencing complex (RISC). DGRC (DiGeorge syndrome critical region gene 8) and TRBP (TAR (HIV) RNA binding protein 2) are double-stranded RNA-binding proteins that facilitate mature miRNA biogenesis by Drosha and Dicer RNase III enzymes, respectively. The guide strand of the miRNA duplex gets incorporated into the effector complex RISC, which recognises specific targets through imperfect base-pairing and induces post-transcriptional gene silencing. Several mechanisms have been proposed for this mode of regulation: miRNAs can induce the repression of translation initiation, mark target mRNAs for degradation by deadenylation, or sequester targets into the cytoplasmic P-body.

On the other hand, if only the seed is perfectly complementary to the target mRNA but the remaining bases show incomplete pairing, RNAi acts through multiple mechanisms leading to translational repression. Eukaryotic mRNA degradation mainly occurs through the shortening of the polyA tail at the 3′ end of the mRNA, and de-capping at the 5′ end, followed by 5′-3′ exonuclease digestion and accumulation of the miRNA in discrete cytoplasmic areas, the so called P-bodies, enriched in components of the mRNA decay pathway.

According to the present invention, expression of the agent and/or interleukin, such as IL-2, may be regulated by endogenous miRNAs using corresponding miRNA target sequences. Using this method, a miRNA endogenously expressed in a cell prevents or reduces transgene expression in that cell by binding to its corresponding miRNA target sequence positioned in the vector or polynucleotide (Brown, B. D. et al. (2007) Nat Biotechnol 25: 1457-1467).

miRNA target sequences that are useful in the present invention include miRNA target sequences which are expressed in haematopoietic cells.

Preferably, the target sequence is the target of an miRNA selected from the group consisting of miR-142, miR-155 and miR-223.

In some embodiments, the nucleotide sequence encoding the agent and/or interleukin is operably linked to one or more miR-142, miR-155 and/or miR-223 target sequences. In preferred embodiments, the nucleotide sequence is operably linked to one or more miR-142 target sequences.

An example miR-142 target sequence is:

(SEQ ID NO: 10)
TCCATAAAGTAGGAAACACTACA

An example miR-155 target sequence is:

(SEQ ID NO: 11)
CCCCTATCACGATTAGCATTAA

An example miR-223 target sequence is:

(SEQ ID NO: 12)
GGGGTATTTGACAAACTGACA

More than one copy of an miRNA target sequence included in the vector may increase the effectiveness of the system. Also it is envisaged that different miRNA target sequences could be included. For example, vectors which express more than one transgene may have the transgene under control of more than one miRNA target sequence, which may or may not be different. The miRNA target sequences may be in tandem, but other arrangements are envisaged. Preferably, the nucleotide sequence or vector comprises 1, 2, 3, 4, 5, 6, 7 or 8 copies of the same or different miRNA target sequence. In preferred embodiments, the nucleotide sequence or vector comprises 4 miR-142 target sequences.

In some embodiments, the target sequence is fully or partially complementary to the miRNA. The term “fully complementary”, as used herein, may mean that the target sequence has a nucleic acid sequence which is 100% complementary to the sequence of the miRNA which recognises it. The term “partially complementary”, as used herein, may mean that the target sequence is only in part complementary to the sequence of the miRNA which recognises it, whereby the partially complementary sequence is still recognised by the miRNA. In other words, a partially complementary target sequence in the context of the present invention is effective in recognising the corresponding miRNA and effecting prevention or reduction of transgene expression in cells expressing that miRNA.

Copies of miRNA target sequences may be separated by a spacer sequence. The spacer sequence may comprise, for example, at least one, at least two, at least three, at least four or at least five nucleotide bases.

### Further Regulatory Elements

The nucleotide sequence or vector of the invention may also comprise one or more additional regulatory sequences with may act pre- or post-transcriptionally. The regulatory sequence may be part of the native transgene locus or may be a heterologous regulatory sequence. The nucleotide sequence or vector of the invention may comprise portions of the or 3′-UTR from the native transgene transcript.

Regulatory sequences are any sequences which facilitate expression of the transgene, i.e. act to increase expression of a transcript, improve nuclear export of mRNA or enhance its stability. Such regulatory sequences include for example post-transcriptional regulatory elements and polyadenylation sites.

A preferred post-transcriptional regulatory element for use in a nucleotide sequence or vector of the invention is the woodchuck hepatitis post-transcriptional regulatory element (WPRE) or a variant thereof.

An example WPRE sequence is:

(SEQ ID NO: 7)
ATCAACCTCTGGATTACAAAATTTGTGAAAGATTGACTGGTATTCTTA
ACTATGTTGCTCCTTTTACGCTATGTGGATACGCTGCTTTAATGCCTT
TGTATCATGCTATTGCTTCCCGTATGGCTTTCATTTTCTCCTCCTTGT
ATAAATCCTGGTTGCTGTCTCTTTATGAGGAGTTGTGGCCCGTTGTCA
GGCAACGTGGCGTGGTGTGCACTGTGTTTGCTGACGCAACCCCCACTG
GTTGGGGCATTGCCACCACCTGTCAGCTCCTTTCCGGGACTTTCGCTT
TCCCCCTCCCTATTGCCACGGCGGAACTCATCGCCGCCTGCCTTGCCC
GCTGCTGGACAGGGGCTCGGCTGTTGGGCACTGACAATTCCGTGGTGT
TGTCGGGGAAATCATCGTCCTTTCCTTGGCTGCTCGCCTGTGTTGCCA
CCTGGATTCTGCGCGGGACGTCCTTCTGCTACGTCCCTTCGGCCCTCA
ATCCAGCGGACCTTCCTTCCCGCGGCCTGCTGCCGGCTCTGCGGCCTC
TTCCGCGTCTTCGCCTTCGCCCTCAGACGAGTCGGATCTCCCTTTGGG
CCGCCTCCCCGC

The invention encompasses the use of any variant sequence of the WPRE which increases expression of the transgene compared to a nucleotide sequence or vector without a WPRE.

### Vectors

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid and/or facilitating the expression of the protein encoded by a segment of nucleic acid.

Vectors comprising polynucleotides used in the invention may be introduced into cells using a variety of techniques known in the art, such as transfection, transduction and transformation.

Transfection may refer to a general process of incorporating a nucleic acid into a cell and includes a process using a non-viral vector to deliver a polynucleotide to a cell. Transduction may refer to a process of incorporating a nucleic acid into a cell using a viral vector.

The vector of the invention may be adapted for liver-specific expression of the nucleotide sequence encoding the agent and/or interleukin.

The term “adapted for liver-specific expression”, as used herein, may refer to preferential expression of the nucleotide sequence in liver tissue, preferably hepatocytes, in comparison to other tissue of a subject. Preferably, no or substantially no expression of the nucleotide sequence occurs in non-liver tissue. The skilled person is readily able to determine expression profiles of a nucleotide sequence using methods known in the art, for example analysing protein and/or mRNA levels in specific cell types obtained from a subject using techniques such as Western blot.

A vector adapted for liver-specific expression may comprise suitable liver-specific expression control sequences, for example as disclosed herein, and/or may be in a form that preferentially transfects, transduces or transforms liver cells, such as hepatocytes.

Preferably, the vector is a viral vector. The vectors of the invention are preferably lentiviral vectors, although it is contemplated that other viral vectors may be used.

Preferably, the viral vector for use according to the invention is in the form of a viral vector particle.

In some embodiments, the vector is an RNA (e.g. mRNA) vector.

Transduction of cells with RNA vectors can be achieved, for example, using liposomes or lipid nanoparticles. In some embodiments, the RNA vector is in the form of a liposome or lipid nanoparticle.

Liposomes may naturally preferentially target hepatocytes. Thus, a vector in the form of a liposome may be adapted for liver-specific expression in the absence of liver-specific expression control sequences. However, it is envisaged that a vector in the form of a liposome may suitably comprise one or more liver-specific expression control sequences, preferably one or more miR-142, miR-155 and/or miR-223 target sequences, preferably further a hepatocyte-specific promoter and/or enhancer.

Lipid nanoparticles may be modified to preferentially target hepatocytes, for example the lipid nanoparticles may comprise a hepatocyte-specific ligand, such as N-acetyl-D-galactosamine (GalNAc).

### Retroviral and Lentiviral Vectors

A retroviral vector may be derived from or may be derivable from any suitable retrovirus. A large number of different retroviruses have been identified. Examples include murine leukaemia virus (MLV), human T cell leukaemia virus (HTLV), mouse mammary tumour virus (MMTV), Rous sarcoma virus (RSV), Fujinami sarcoma virus (FuSV), Moloney murine leukaemia virus (Mo-MLV), FBR murine osteosarcoma virus (FBR MSV), Moloney murine sarcoma virus (Mo-MSV), Abelson murine leukaemia virus (A-MLV), avian myelocytomatosis virus-29 (MC29) and avian erythroblastosis virus (AEV). A detailed list of retroviruses may be found in Coffin, J. M. et al. (1997) Retroviruses, Cold Spring Harbour Laboratory Press, 758-63.

Retroviruses may be broadly divided into two categories, “simple” and “complex”. Retroviruses may be even further divided into seven groups. Five of these groups represent retroviruses with oncogenic potential. The remaining two groups are the lentiviruses and the spumaviruses.

The basic structure of retrovirus and lentivirus genomes share many common features such as a 5′ LTR and a 3′ LTR. Between or within these are located a packaging signal to enable the genome to be packaged, a primer binding site, integration sites to enable integration into a host cell genome, and gag, pol and env genes encoding the packaging components—these are polypeptides required for the assembly of viral particles. Lentiviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration and transcription. LTRs also serve as enhancer-promoter sequences and can control the expression of the viral genes.

The LTRs themselves are identical sequences that can be divided into three elements: U3, R and U5. U3 is derived from the sequence unique to the 3′ end of the RNA. R is derived from a sequence repeated at both ends of the RNA. U5 is derived from the sequence unique to the 5′ end of the RNA. The sizes of the three elements can vary considerably among different retroviruses.

In a defective retroviral vector genome gag, pol and env may be absent or not functional.

In a typical retroviral vector, at least part of one or more protein coding regions essential for replication may be removed from the virus. This makes the viral vector replication-defective. Portions of the viral genome may also be replaced by a library encoding candidate modulating moieties operably linked to a regulatory control region and a reporter moiety in the vector genome in order to generate a vector comprising candidate modulating moieties which is capable of transducing a target host cell and/or integrating its genome into a host genome.

Lentivirus vectors are part of the larger group of retroviral vectors. A detailed list of lentiviruses may be found in Coffin, J. M. et al. (1997) Retroviruses, Cold Spring Harbour Laboratory Press, 758-63. In brief, lentiviruses can be divided into primate and non-primate groups. Examples of primate lentiviruses include but are not limited to human immunodeficiency virus (HIV), the causative agent of human acquired immunodeficiency syndrome (AIDS); and simian immunodeficiency virus (SIV). Examples of non-primate lentiviruses include the prototype “slow virus” visna/maedi virus (VMV), as well as the related caprine arthritis-encephalitis virus (CAEV), equine infectious anaemia virus (EIAV), and the more recently described feline immunodeficiency virus (FIV) and bovine immunodeficiency virus (BIV).

The lentivirus family differs from retroviruses in that lentiviruses have the capability to infect both dividing and non-dividing cells (Lewis, Pet al. (1992) EMBO J. 11: 3053-8; Lewis, P. F. et al. (1994) J. Virol. 68: 510-6). In contrast, other retroviruses, such as MLV, are unable to infect non-dividing or slowly dividing cells such as those that make up, for example, muscle, brain, lung and liver tissue.

A lentiviral vector, as used herein, is a vector which comprises at least one component part derivable from a lentivirus. Preferably, that component part is involved in the biological mechanisms by which the vector infects cells, expresses genes or is replicated.

The lentiviral vector may be a “primate” vector. The lentiviral vector may be a “non-primate” vector (i.e. derived from a virus which does not primarily infect primates, especially humans). Examples of non-primate lentiviruses may be any member of the family of lentiviridae which does not naturally infect a primate.

As examples of lentivirus-based vectors, HIV-1- and HIV-2-based vectors are described below.

In preferred embodiments, the vector is an HIV vector, such as a HIV-1 or HIV-2 vector, preferably a HIV-1 vector.

The HIV-1 vector contains cis-acting elements that are also found in simple retroviruses. It has been shown that sequences that extend into the gag open reading frame are important for packaging of HIV-1. Therefore, HIV-1 vectors often contain the relevant portion of gag in which the translational initiation codon has been mutated. In addition, most HIV-1 vectors also contain a portion of the env gene that includes the RRE. Rev binds to RRE, which permits the transport of full-length or singly spliced mRNAs from the nucleus to the cytoplasm. In the absence of Rev and/or RRE, full-length HIV-1 RNAs accumulate in the nucleus. Alternatively, a constitutive transport element from certain simple retroviruses such as Mason-Pfizer monkey virus can be used to relieve the requirement for Rev and RRE. Efficient transcription from the HIV-1 LTR promoter requires the viral protein Tat.

Most HIV-2-based vectors are structurally very similar to HIV-1 vectors. Similar to HIV-1-based vectors, HIV-2 vectors also require RRE for efficient transport of the full-length or singly spliced viral RNAs.

Preferably, the viral vector used in the present invention has a minimal viral genome.

By “minimal viral genome” it is to be understood that the viral vector has been manipulated so as to remove the non-essential elements and to retain the essential elements in order to provide the required functionality to infect, transduce and deliver a nucleotide sequence of interest to a target host cell. Further details of this strategy can be found in WO 1998/017815.

Preferably, the plasmid vector used to produce the viral genome within a host cell/packaging cell will have sufficient lentiviral genetic information to allow packaging of an RNA genome, in the presence of packaging components, into a viral particle which is capable of infecting a target cell, but is incapable of independent replication to produce infectious viral particles within the final target cell. Preferably, the vector lacks a functional gag-pol and/or env gene and/or other genes essential for replication.

However, the plasmid vector used to produce the viral genome within a host cell/packaging cell will also include transcriptional regulatory control sequences operably linked to the lentiviral genome to direct transcription of the genome in a host cell/packaging cell. These regulatory sequences may be the natural sequences associated with the transcribed viral sequence (i.e. the 5′ U3 region), or they may be a heterologous promoter, such as another viral promoter (e.g. the CMV promoter).

The vectors may be self-inactivating (SIN) vectors in which the viral enhancer and promoter sequences have been deleted. SIN vectors can be generated and transduce non-dividing cells in vivo with an efficacy similar to that of wild-type vectors. The transcriptional inactivation of the long terminal repeat (LTR) in the SIN provirus should prevent mobilisation by replication-competent virus. This should also enable the regulated expression of genes from internal promoters by eliminating any cis-acting effects of the LTR.

In some embodiments, the vector is an integration-defective lentiviral vector (IDLV).

Integration defective lentiviral vectors (IDLVs) can be produced, for example, either by packaging the vector with catalytically inactive integrase (such as an HIV integrase bearing the D64V mutation in the catalytic site; Naldini, L. et al. (1996) Science 272: 263-7; Naldini, L. et al. (1996) Proc. Natl. Acad. Sci. USA 93: 11382-8; Leavitt, A. D. et al. (1996) J. Virol. 70: 721-8) or by modifying or deleting essential att sequences from the vector LTR (Nightingale, S. J. et al. (2006) Mol. Ther. 13: 1121-32), or by a combination of the above.

### Adeno-Associated Viral (AAV) Vectors

Adeno-associated virus (AAV) has a high frequency of integration and it can infect non-dividing cells. This makes it useful for delivery of genes into mammalian cells in tissue culture.

AAV has a broad host range for infectivity. Details concerning the generation and use of AAV vectors are described in U.S. Pat. Nos. 5,139,941 and 4,797,368.

Recombinant AAV vectors have been used successfully for in vitro and in vivo transduction of marker genes and genes involved in human diseases.

### Adenoviral Vectors

The adenovirus is a double-stranded, linear DNA virus that does not go through an RNA intermediate. There are over 50 different human serotypes of adenovirus divided into 6 subgroups based on the genetic sequence homology. The natural targets of adenovirus are the respiratory and gastrointestinal epithelia, generally giving rise to only mild symptoms. Serotypes 2 and 5 (with 95% sequence homology) are most commonly used in adenoviral vector systems and are normally associated with upper respiratory tract infections in the young.

Adenoviruses have been used as vectors for gene therapy and for expression of heterologous genes. The large (36 kb) genome can accommodate up to 8 kb of foreign insert DNA and is able to replicate efficiently in complementing cell lines to produce very high titres of up to 10¹². Adenovirus is thus one of the best systems to study the expression of genes in primary non-replicative cells.

The expression of viral or foreign genes from the adenovirus genome does not require a replicating cell. Adenoviral vectors enter cells by receptor mediated endocytosis. Once inside the cell, adenovirus vectors rarely integrate into the host chromosome. Instead, they function episomally (independently from the host genome) as a linear genome in the host nucleus. Hence the use of recombinant adenovirus alleviates the problems associated with random integration into the host genome.

### Arenoviral Vectors

The arenavirus is enveloped and has a segmented RNA genome consisting of two single-stranded ambisense RNAs (L and S molecules). The S (short) segment contains the glycoprotein (GP) precursor (GPC) genes, GP-1 and GP-2, and the nucleoprotein (NP) gene. The GP protein is important for viral cell entry and viral propagation.

Arenaviruses can infect rodents and humans; at least eight arenaviruses are known to cause human disease that range in severity. Lymphocytic choriomeningitis virus (LCMV) is a rodent-borne virus that causes lymphocytic choriomeningitis, which can present as mild febrile illness or more severe neurological disease. LCMV exhibits natural tropism for dendritic cells.

Replication-defective LCMV vectors can be created by the mutation or substitution of the GP gene(s), which renders the virus propagation-incompetent in vivo and in vitro. Recombinant, replication-defective LCMV (rLCMV) cannot enter new host cells, alleviating problems associated with off-target effects of viral vector-based gene editing.

### Protein Transduction

As an alternative to the delivery of polynucleotides, such as using vectors, the agent and/or interleukin of the invention may be delivered to cells as proteins, such as by protein transduction.

Protein transduction may be via vector delivery (Cai, Y. et al. (2014) Elife 3: e01911; Maetzig, T. et al. (2012) Curr. Gene Ther. 12: 389-409). Vector delivery involves the engineering of viral particles (e.g. lentiviral particles) to comprise the proteins to be delivered to a cell. Accordingly, when the engineered viral particles enter a cell as part of their natural life cycle, the proteins comprised in the particles are carried into the cell.

Protein transduction may be via protein delivery (Gaj, T. et al. (2012) Nat. Methods 9: 805-7). Protein delivery may be achieved, for example, by utilising a vehicle (e.g. a nanoparticle).

In some embodiments, the interleukin is in complex with a an anti-interleukin antibody, preferably a non-neutralising antibody. In some embodiments, the IL-2 is in complex with an anti-IL-2 antibody. In some embodiments, the IL-7 is in complex with an anti-IL-7 antibody. In some embodiments, the IL-15 is in complex with an anti-IL-15 antibody.

In some embodiments, the agent and/or interleukin is comprised in a nanoparticle, such as a liposome.

In one aspect, the invention provides an agent that inhibits GM-CSF which is adapted to be targeted to the liver.

The term “adapted to be targeted to the liver”, as used herein, may refer to preferential delivery of the agent and/or interleukin to liver tissue, preferably hepatocytes, in comparison to other tissue of a subject. Preferably, no or substantially no agent or interleukin targeted in said way is delivered to or accumulated in non-liver tissue. The skilled person is readily able to determine delivery profiles using methods known in the art, for example analysing protein levels in specific cell types obtained from a subject using techniques such as Western blot.

Targeting to the liver may be achieved for example using nanoparticles that are adapted to be targeted to the liver.

The agent, interleukin and/or nanoparticle may be, for example, adapted to be targeted to a specific liver cell type. In some embodiments, the targeting is to hepatocytes. In some embodiments, the targeting is to liver sinusoidal endothelial cells. In some embodiments, the targeting is to Kupffer cells. In some embodiments, the targeting is to Type 2 Kupffer cells.

In some embodiments, the nanoparticle comprises a liver-specific ligand. The liver-specific ligand may be, for example, a hepatocyte-, liver sinusoidal endothelial cell- or Kupffer cell-specific ligand. The liver-specific ligand may be, for example, a Type 2 Kupffer cell-specific ligand. The cell-specific ligand may be an antibody that binds to a marker expressed by the cell. For example, a Type 2 Kupffer cell-specific ligand may be an antibody that binds to a Type 2 Kupffer cell marker (such as a KC2 marker disclosed herein). In preferred embodiments, the Type 2 Kupffer cell-specific ligand is an anti-CD206 or anti-Mrc1 antibody.

Examples of suitable ligands and their target liver cell type, and further means of targeting nanoparticles (e.g. passive targeting means) are described in the table below.

| | | Targeting ligand or | |
|---|---|---|---|
| Liver cell type | Cellular target | means | Reference |
| Hepatic | Mannose-6- | Mannose-6-phosphate | M. Moreno, T. Gonzalo, |
| stellate | phosphate | | R. J. Kok, P. Sancho-Bru, |
| cells | receptor | | M. van Beuge, J. Swart, et |
| | | | al., Hepatology 51 (2010) |
| | | | 942; N. Yang, Z. Ye, F. Li, |
| | | | R. I. Mahato, Bioconjug. |
| | | | Chem. 20 (2009) 213; W. I. |
| | | | Hagens, A. Mattos, R. |
| | | | Greupink, A. de Jager- |
| | | | Krikken, C. Reker-Smit, A. |
| | | | van Loenen-Weemaes, et |
| | | | al., Pharm. Res. 24 (2007) |
| | | | 566; J. E. Adrian, K. |
| | | | Poelstra, G. L. Scherphof, |
| | | | G. Molema, D. K. Meijer, |
| | | | C. Reker-Smit, et al., J. |
| | | | Hepatol. 44 (2006) 560; |
| | | | J. E. Adrian, J. A. Kamps, |
| | | | G. L. Scherphof, D. K. |
| | | | Meijer, A. M. van Loenen- |
| | | | Weemaes, C. Reker-Smit, |
| | | | et al., Biochim. Biophys. |
| | | | Acta 1768 (2007) 1430; |
| | | | J. E. Adrian, K. Poelstra, |
| | | | G. L. Scherphof, D. K. |
| | | | Meijer, A. M. van Loenen- |
| | | | Weemaes, C. Reker-Smit, |
| | | | et al., J. Pharmacol. Exp. |
| | | | Ther. 321 (2007) 536; Z. |
| | | | Ye, K. Cheng, R.V. |
| | | | Guntaka, R. I. Mahato, |
| | | | Biochemistry (Mosc). 44 |
| | | | (2005) 4466. |
| | Retinol binding | Vitamin A | Y. Sato, K. Murase, J. |
| | protein | | Kato, M. Kobune, T. Sato, |
| | receptor | | Y. Kawano, et al., Nat. |
| | | | Biotechnol. 26 (2008) 431. |
| | Type VI | Cyclic RGD | L. Beljaars, G. Molema, D. |
| | collagen | | Schuppan, A. Geerts, P. J. |
| | receptor | | De Bleser, B. Weert, et al., |
| | | | J. Biol. Chem. 275 (2000) |
| | | | 12743; S. L. Du, H. Pan, |
| | | | W. Y. Lu, J. Wang, J. Wu, |
| | | | J. Y. Wang, J. Pharmacol. |
| | | | Exp. Ther. 322 (2007) |
| | | | 560; F. Li, J. Y. Sun, J. Y. |
| | | | Wang, S. L. Du, W. Y. Lu, |
| | | | M. Liu, et al., J. Control. |
| | | | Release 131 (2008) 77. |
| | PDGF receptor | PDGF | W. I. Hagens, A. Mattos, R. |
| | | | Greupink, A. de Jager- |
| | | | Krikken, C. Reker-Smit, A. |
| | | | van Loenen-Weemaes, et |
| | | | al., Pharm. Res. 24 (2007) |
| | | | 566. |
| | Scavenger | Human | J. E. Adrian, K. Poelstra, |
| | receptor | serum | G. L. Scherphof, G. |
| | class | albumin | Molema, D. K. Meijer, C. |
| | A | | Reker-Smit, et al., J. |
| | | | Hepatol. 44 (2006) 560; |
| | | | J. E. Adrian, J. A. Kamps, |
| | | | G. L. Scherphof, D. K. |
| | | | Meijer, A. M. van Loenen- |
| | | | Weemaes, C. Reker-Smit, |
| | | | et al., Biochim. Biophys. |
| | | | Acta 1768 (2007) 1430; |
| | | | J. E. Adrian, K. Poelstra, |
| | | | G. L. Scherphof, D. K. |
| | | | Meijer, A. M. van Loenen- |
| | | | Weemaes, C. Reker-Smit, |
| | | | et al., J. Pharmacol. Exp. |
| | | | Ther. 321 (2007) 536. |
| Hepatocytes | Asialoglycoprotein | Asialoorosomucoid | B. T. Kren, G. M. Unger, L. |
| | receptor | | Sjeklocha, A. A. Trossen, |
| | | | V. Korman, B. M. Diethelm- |
| | | | Okita, et al., J. Clin. Invest. |
| | | | 119 (2009) 2086. |
| | | Galactoside | F. Suriano, R. Pratt, J. P. |
| | | | Tan, N. Wiradharma, A. |
| | | | Nelson, Y. Y. Yang, et al., |
| | | | Biomaterials 31 (2010) |
| | | | 2637; T. Terada, M. Iwai, |
| | | | S. Kawakami, F. |
| | | | Yamashita, M. Hashida, J. |
| | | | Control. Release 111 |
| | | | (2006) 333. |
| | | Galactosamine | L. W. Seymour, D. R. Ferry, |
| | | | D. Anderson, S. |
| | | | Hesslewood, P. J. Julyan, |
| | | | R. Poyner, et al., J. Clin. |
| | | | Oncol. 20 (2002) 1668; Y. |
| | | | Cao, Y. Gu, H. Ma, J. Bai, |
| | | | L. Liu, P. Zhao, et al., Int. |
| | | | J. Biol. Macromol. 46 |
| | | | (2010) 245; Y. C. Wang, |
| | | | X. Q. Liu, T. M. Sun, M. H. |
| | | | Xiong, J. Wang, J. Control. |
| | | | Release 128 (2008) 32. |
| | | Asialofetuin | S. Diez, G. Navarro, I. C. T. |
| | | | de, J. Gene Med. 11 |
| | | | (2009) 38. |
| | | Sterylglucoside | X. R. Qi, W. W. Yan, J. Shi, |
| | | | World J. Gastroenterol. 11 |
| | | | (2005) 4947. |
| | | Lactose/lactobionic acid | Z. Xu, L. Chen, W. Gu, Y. |
| | | | Gao, L. Lin, Z. Zhang, et |
| | | | al., Biomaterials 30 (2009) |
| | | | 226; Y. Kato, H. Onishi, Y. |
| | | | Machida, Int. J. Pharm. |
| | | | 226 (2001) 93; K. W. Yang, |
| | | | X. R. Li, Z. L. Yang, P. Z. Li, |
| | | | F. Wang, Y. Liu, J. |
| | | | Biomed. Mater. Res. A 88 |
| | | | (2009) 140; Q. Wang, L. |
| | | | Zhang, W. Hu, Z. H. Hu, |
| | | | Y. Y. Bei, J. Y. Xu, et al., |
| | | | Nanomedicine 6 (2010) |
| | | | 371. |
| | | PVLA | C. S. Cho, A. Kobayashi, |
| | | | R. Takei, T. Ishihara, A. |
| | | | Maruyama, T. Akaike, |
| | | | Biomaterials 22 (2001) 45; |
| | | | Y. Watanabe, X. Liu, I. |
| | | | Shibuya, T. Akaike, J. |
| | | | Biomater. Sci. Polym. Ed. |
| | | | 11 (2000) 833. |
| | Scavenger | Apolipoprotein | S. I. Kim, D. Shin, T. H. |
| | receptor class | A-I | Choi, J. C. Lee, G. J. |
| | B type I | | Cheon, K. Y. Kim, et al., |
| | | | Mol. Ther. 15 (2007) 1145; |
| | | | S. I. Kim, D. Shin, H. Lee, |
| | | | B. Y. Ahn, Y. Yoon, M. |
| | | | Kim, J. Hepatol. 50 (2009) |
| | | | 479; M. Feng, Q. Cai, H. |
| | | | Huang, P. Zhou, Eur. J. |
| | | | Pharm. Biopharm. 68 |
| | | | (2008) 688. |
| | Plasma | Linoleic acid | S. J. Cheong, C. M. Lee, |
| | membrane | | S. L. Kim, H. J. Jeong, E. M. |
| | fatty acid | | Kim, E. H. Park, et al., Int. |
| | binding protein | | J. Pharm. 372 (2009) 169; |
| | (Putative) | | C. M. Lee, H. J. Jeong, S. L. |
| | | | Kim, E. M. Kim, D. W. Kim, |
| | | | S. T. Lim, et al., Int. J. |
| | | | Pharm. 371 (2009) 163. |
| | Glycyrrhizin | Glycyrrhizin | S. J. Mao, S. X. Hou, R. He, |
| | receptors | | L. K. Zhang, D. P. Wei, |
| | | | Y. Q. Bi, et al., World J. |
| | | | Gastroenterol. 11 (2005) |
| | | | 3075. |
| | Heparan | Acety- | K. J. Longmuir, S. M. |
| | sulfate | ICKNEKKNKIERNNKLKQPP- | Haynes, J. L. Baratta, N. |
| | | amide | Kasabwalla, R. T. |
| | | | Robertson, Int. J. Pharm. |
| | | | 382 (2009) 222. |
| | IL-6-receptor | Pre-S1 | R. Miyata, M. Ueda, H. |
| | and/or | | Jinno, T. Konno, K. |
| | immunoglobulin | | Ishihara, N. Ando, et al., |
| | A binding | | Int. J. Cancer 124 (2009) |
| | protein | | 2460. |
| | (Putative) | | |
| Macrophages | Macrophage | | Fakhrul Ahsan, Isabel P. |
| (including | receptors (Fc | | Rivas, Mansoor A. Khan, |
| Kupffer | receptors, | | Ana I. Torres Suarez. |
| cells, | complement, | | Targeting to |
| splenic | fibronectin | | macrophages: role of |
| macrophages, | lipoprotein, | | physicochemical |
| etc . . .) | mannosyl, | | properties of particulate |
| | galactosyl) | | carriers-liposomes and |
| | | | microspheres-on the |
| | | | phagocytosis by |
| | | | macrophages. Journal of |
| | | | Controlled Release 79 |
| | | | (2002) 29-40. |
| | Passive | Positively charged and | R. A. Schwendener, P. A. |
| | targeting | large sized Liposomes | Lagocki, Y. E. Rahman, |
| | | | The effects of charge and |
| | | | size on the interaction of |
| | | | unilamellar liposomes with |
| | | | macrophages, Biochim. |
| | | | Biophys. Acta 7721992, |
| | | | pp. 195-200. (1984) |
| | | | 93-101; Y. E. Rahman, E. A. |
| | | | Cernry, K. R. Patel, E. H. |
| | | | Lau, B. J. Wright, |
| | | | Differential uptake of |
| | | | liposomes varying in size |
| | | | and lipid composition by |
| | | | parenchymal and kupffer |
| | | | cells of mouse liver, Life |
| | | | Sci. 31 (1982) 2061-2071. |
| | | Inclusion of negatively | Fakhrul Ahsan, Isabel P. |
| | | charged phospholipids | Rivas, Mansoor A. Khan, |
| | | such as | Ana I. Torres Suarez. |
| | | phosphatidylserine and | Targeting to |
| | | phosphatidylglycerol | macrophages: role of |
| | | Peptide grafted liposomes | physicochemical |
| | | Hydrophobic and large | properties of particulate |
| | | sized polymeric | carriers-liposomes and |
| | | microspheres | microspheres-on the |
| | | Coating of polymeric | phagocytosis by |
| | | microspheres with opsonic | macrophages. Journal of |
| | | materials (gamma- | Controlled Release 79 |
| | | globulin, human | (2002) 29-40. |
| | | fibronectin, bovine tuftsin, | |
| | | and gelatin) | |

### T Cells

T cells (or T lymphocytes) are a type of lymphocyte that play a central role in cell-mediated immunity. They can be distinguished from other lymphocytes, such as B cells and natural killer cells (NK cells), by the presence of a T cell receptor (TCR) on the cell surface.

The T cells used in the present invention may be used for adoptive T cell transfer.

The term “adoptive T cell transfer”, as used herein, refers to the administration of a T cell population to a patient. A T cell may be isolated from a subject and then genetically modified and cultured in vitro (ex vivo) in order to express a TCR or chimeric antigen receptor (CAR) before being administered to a patient.

Adoptive cell transfer may be allogenic or autologous.

By “autologous cell transfer” it is to be understood that the starting population of cells is obtained from the same subject as that to which the T cell population is administered. Autologous transfer is advantageous as it avoids problems associated with immunological incompatibility and is available to subjects irrespective of the availability of a genetically matched donor.

By “allogeneic cell transfer” it is to be understood that the starting population of cells is obtained from a different subject as that to which the T cell population is administered. Preferably, the donor will be genetically matched to the subject to which the cells are administered to minimise the risk of immunological incompatibility. Alternatively, the donor may be mismatched and unrelated to the patient.

Suitable doses of transduced cell populations are such as to be therapeutically and/or prophylactically effective. The dose to be administered may depend on the subject and condition to be treated, and may be readily determined by a skilled person.

The T cell may be derived from a T cell isolated from a patient. The T cell may be part of a mixed cell population isolated from the subject, such as a population of peripheral blood lymphocytes (PBL). T cells within the PBL population may be activated by methods known in the art, such as using anti-CD3 and/or anti-CD28 antibodies or cell sized beads conjugated with anti-CD3 and/or anti-CD28 antibodies.

The T cell may be a CD4⁺ helper T cell or a CD8⁺ cytotoxic T cell. The T cell may be in a mixed population of CD4⁺ helper T cells/CD8⁺ cytotoxic T cells. Polyclonal activation, for example using anti-CD3 antibodies optionally in combination with anti-CD28 antibodies may trigger the proliferation of CD4⁺ and CD8⁺ T cells.

A T cell may be isolated from the subject to which the population of T cells is to be adoptively transferred. In this respect, the cell may be made by isolating a T cell from a subject, optionally activating the T cell, optionally transferring a TCR- or CAR-encoding gene to the cell ex vivo. Subsequent immunotherapy of the subject may then be carried out by adoptive transfer of the population of cells.

Alternatively the T cell may be derived from a stem cell, such as a haemopoietic stem cell (HSC). Gene transfer into HSCs does not lead to TCR expression at the cell surface as stem cells do not express CD3 molecules. However, when stem cells differentiate into lymphoid precursors that migrate to the thymus, the initiation of CD3 expression leads to the surface expression of the TCR in thymocytes.

An advantage of this approach is that the mature T cells, once produced, express only an introduced TCR and little or no endogenous TCR chains, because the expression of the introduced TCR chains suppresses rearrangement of endogenous TCR gene segments to form functional TCR alpha and beta genes. A further benefit is that the gene-modified stem cells are a continuous source of mature T cells with the desired antigen specificity. Accordingly, the vector as defined herein may be used in combination with a gene-modified stem cell, preferably a gene-modified hematopoietic stem cell, which, upon differentiation, produces a T cell.

Other approaches known in the art may be used to reduce, limit, prevent, silence, or abrogate expression of endogenous genes in the cells of the present invention or cells prepared by the methods of the present invention.

The term “disrupting”, as used herein, refers to reducing, limiting, preventing, silencing or abrogating expression of a gene. The skilled person is able to use any method known in the art to disrupt an endogenous gene, e.g. any suitable method for genome editing, gene silencing, gene knock-down or gene knock-out.

For example, an endogenous gene may be disrupted with an artificial nuclease. An artificial nuclease is, e.g. an artificial restriction enzyme engineered to selectively target a specific polynucleotide sequence (e.g. encoding a gene of interest) and induce a double strand break in said polynucleotide sequence. Typically, the double strand break (DSB) will be repaired by error-prone non-homologous end joining (NHEJ) thereby resulting in the formation of a non-functional polynucleotide sequence, which may be unable to express an endogenous gene.

In some embodiments, the artificial nuclease is selected from the group consisting of zinc finger nucleases (ZFNs), transcription activator-like effector nucleases (TALENs) and CRISPR/Cas (e.g. CRISPR/Cas9).

### T Cell Receptor (TCR)

During antigen processing, antigens are degraded inside cells and then carried to the cell surface by major histocompatibility complex (MHC) molecules. T cells are able to recognise this peptide:MHC complex at the surface of the antigen presenting cell. There are two different classes of MHC molecules: MHC I and MHC II, each class delivers peptides from different cellular compartments to the cell surface.

A T cell receptor (TCR) is a molecule found on the surface of T cells that is responsible for recognising antigens bound to MHC molecules. The TCR heterodimer consists of an alpha (α) and beta (β) chain in around 95% of T cells, whereas around 5% of T cells have TCRs consisting of gamma (γ) and delta (δ) chains.

Engagement of the TCR with antigen and MHC results in activation of the T lymphocyte on which the TCR is expressed through a series of biochemical events mediated by associated enzymes, co-receptors and specialised accessory molecules.

Each chain of the TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end.

The variable domain of both the TCR α chain and β chain have three hypervariable or complementarity determining regions (CDRs). CDR3 is the main CDR responsible for recognising processed antigen, although CDR1 of the alpha chain has also been shown to interact with the N-terminal part of the antigenic peptide, whereas CDR1 of the beta chain interacts with the C-terminal part of the peptide. CDR2 is thought to recognise the MHC molecule.

The constant domain of the TCR domain consists of short connecting sequences in which a cysteine residue forms a disulfide bond, making a link between the two chains.

The TCR used in the present invention may have one or more additional cysteine residues in each of the α and β chains such that the TCR may comprise two or more disulphide bonds in the constant domains.

The structure allows the TCR to associate with other molecules like CD3 which possess three distinct chains (γ, δ, and ε) in mammals and the ζ-chain. These accessory molecules have negatively charged transmembrane regions and are vital to propagating the signal from the TCR into the cell. The CD3- and ζ-chains, together with the TCR, form what is known as the T cell receptor complex.

The signal from the T cell complex is enhanced by simultaneous binding of the MHC molecules by a specific co-receptor. For helper T cells, this co-receptor is CD4 (specific for class II MHC); whereas for cytotoxic T cells, this co-receptor is CD8 (specific for class I MHC). The co-receptor allows prolonged engagement between the antigen presenting cell and the T cell and recruits essential molecules (e.g. LCK) inside the cell involved in the signalling of the activated T lymphocyte.

Accordingly, the term “T cell receptor” (TCR), as used herein, refers to a molecule capable of recognising a peptide when presented by an MHC molecule. The molecule may be a heterodimer of two chains α and β (or optionally γ and δ) or it may be a single chain TCR construct.

The TCR used in the present invention may be a hybrid TCR comprising sequences derived from more than one species. For example, it has surprisingly been found that murine TCRs are more efficiently expressed in human T cells than human TCRs. The TCR may therefore comprise human variable regions and murine constant regions.

A disadvantage of this approach is that the murine constant sequences may trigger an immune response, leading to rejection of the transferred T cells. However, the conditioning regimens used to prepare patients for adoptive T cell therapy may result in sufficient immunosuppression to allow the engraftment of T cells expressing murine sequences.

The portion of the TCR that establishes the majority of the contacts with the antigenic peptide bound to the major histocompatibility complex (MHC) is the complementarity determining region 3 (CDR3), which is unique for each T cell clone. The CDR3 region is generated upon somatic rearrangement events occurring in the thymus and involving non-contiguous genes belonging to the variable (V), diversity (D, for β and δ chains) and joining (J) genes. Furthermore, random nucleotides inserted/deleted at the rearranging loci of each TCR chain gene greatly increase diversity of the highly variable CDR3 sequence. Thus, the frequency of a specific CDR3 sequences in a biological sample indicates the abundance of a specific T cell population. The great diversity of the TCR repertoire in healthy human beings provides a wide range protection towards a variety of foreign antigens presented by MHC molecules on the surface of antigen presenting cells. In this regard, it is of note that theoretically up to 10¹⁵ different TCRs can be generated in the thymus.

T cell receptor diversity is focused on CDR3 and this region is primarily responsible for antigen recognition.

TCRs specific for an antigen, such as a virus antigen (e.g. hepatitis virus antigen), bacterial antigen or parasite antigen, may be generated easily by the person skilled in the art using any method known in the art.

Suitable hepatitis virus antigens include hepatitis B virus large envelope protein; hepatitis B virus middle envelope protein; hepatitis B virus small envelope protein; hepatitis B virus core protein; and hepatitis B virus polymerase.

For example, hepatitis virus antigen-specific TCRs may be identified by the TCR gene capture method of Linnemann et al. (Nature Medicine 19: 1534-1541 (2013)). Briefly, this method uses a high-throughput DNA-based strategy to identify TCR sequences by the capture and sequencing of genomic DNA fragments encoding the TCR genes and may be used to identify hepatitis virus antigen-specific TCRs.

### Improved TCR Expression and Reduced TCR Mispairing

Increasing the supply of CD3 molecules may increase TCR expression, for example, in a cell that has been modified to express the TCRs of the present invention. Accordingly, the T cell may be modified (e.g. using a vector) to comprise one or more genes encoding CD3-gamma, CD3-delta, CD3-epsilon and/or CD3-zeta. In some embodiments, the T cell comprises a gene encoding CD3-zeta. The T cell may comprise a gene encoding CD8. The vector encoding such genes may encode a selectable marker or a suicide gene, to increase the safety profile of the genetically engineered cell. The genes may be linked by self-cleaving sequences, such as the 2A self-cleaving sequence.

Alternatively one or more separate vectors encoding a CD3 gene may be provided for co-transfer to a T cell simultaneously, sequentially or separately with one or more vectors encoding TCRs.

The transgenic TCR may be expressed in a T cell used in the present invention to alter the antigen specificity of the T cell. TCR-transduced T cells express at least two TCR alpha and two TCR beta chains. While the endogenous TCR alpha/beta chains form a receptor that is self-tolerant, the introduced TCR alpha/beta chains form a receptor with defined specificity for the given target antigen.

However, TCR gene therapy requires sufficient expression of transferred (i.e. transgenic) TCRs as the transferred TCR might be diluted by the presence of the endogenous TCR, resulting in suboptimal expression of the tumor specific TCR. Furthermore, mispairing between endogenous and introduced chains may occur to form novel receptors, which might display unexpected specificities for self-antigens and cause autoimmune damage when transferred into patients.

Hence, several strategies have been explored to reduce the risk of mispairing between endogenous and introduced TCR chains. Mutations of the TCR alpha/beta interface is one strategy currently employed to reduce unwanted mispairing. For example, the introduction of a cysteine in the constant domains of the alpha and beta chain allows the formation of a disulfide bond and enhances the pairing of the introduced chains while reducing mispairing with wild type chains.

Accordingly, the TCRs used in the present invention may comprise one or more mutations at the α chain/β chain interface, such that when the α chain and the β chain are expressed in a T cell, the frequency of mispairing between said chains and endogenous TCR α and β chains is reduced. In some embodiments, the one or more mutations introduce a cysteine residue into the constant region domain of each of the α chain and the β chain, wherein the cysteine residues are capable of forming a disulphide bond between the α chain and the β chain.

Another strategy to reduce mispairing relies on the introduction of polynucleotide sequences encoding siRNA and designed to limit the expression of the endogenous TCR genes (Okamoto S. (2009) Cancer research 69: 9003-9011).

Accordingly, the vector or polynucleotide encoding the TCRs used in the present invention may comprise one or more siRNA or other agents aimed at limiting or abrogating the expression of the endogenous TCR genes.

It is also possible to combine artificial nucleases, such as zinc finger nucleases (ZFNs), transcription activator-like effector nucleases (TALENs) or CRISPR/Cas systems, designed to target the constant regions of the endogenous TCR genes (TRAC and/or TRBC), to obtain the permanent disruption of the endogenous TCR alpha and/or beta chain genes, thus allowing full expression of the tumor specific TCR and thus reducing or abrogating the risk of TCR mispairing. This process, known as the TCR gene editing proved superior to TCR gene transfer in vitro and in vivo (Provasi E. et al. (2012) Nature Medicine 18: 807-15).

In addition, the genome editing technology allows targeted integration of an expression cassette, comprising a polynucleotide encoding a TCR used in the present invention, and optionally one or more promoter regions and/or other expression control sequences, into an endogenous gene disrupted by the artificial nucleases (Lombardo A. (2007) Nature Biotechnology 25: 1298-1306).

Another strategy developed to increase expression of transgenic TCRs and to reduce TCR mispairing consists in “murinisation,” which replaces the human TCR α and TCR β constant regions (e.g. the TRAC, TRBC1 and TRBC2 regions) by their murine counterparts. Murinisation of TCR constant regions is described in, for example, Sommermeyer et al. (2010) J Immunol 184: 6223-6231. Accordingly, the TCR used in the present invention may be murinised.

### Chimeric Antigen Receptor (CAR)

CARs comprise an extracellular ligand binding domain, most commonly a single chain variable fragment of a monoclonal antibody (scFv) linked to intracellular signaling components, most commonly CD3ζ alone or combined with one or more costimulatory domains. A spacer is often added between the extracellular antigen-binding domain and the transmembrane moiety to optimise the interaction with the target.

A CAR for use in the present invention may comprise:
(i) an antigen-specific targeting domain;
(ii) a transmembrane domain;
(iii) optionally at least one costimulatory domain; and
(iv) an intracellular signaling domain.

Preferably, the antigen-specific targeting domain comprises an antibody or fragment thereof, more preferably a single chain variable fragment.

In some embodiments, the antigen-specific targeting domain targets a hepatitis virus antigen.

In some embodiments, the hepatitis virus antigen is selected from the group consisting of hepatitis B virus large envelope protein; hepatitis B virus middle envelope protein; hepatitis B virus small envelope protein; hepatitis B virus core protein; and hepatitis B virus polymerase.

Examples of transmembrane domains include a transmembrane domain of a zeta chain of a T cell receptor complex, CD28 and CD8a.

Examples of costimulatory domains include costimulating domains from CD28, CD137 (4-1BB), CD134 (OX40), DapIO, CD27, CD2, CD5, ICAM-1, LFA-1, Lck, TNFR-I, TNFR-II, Fas, CD30 and CD40.

In some embodiments, the costimulatory domain is a costimulating domain from CD28.

Examples of intracellular signaling domains include human CD3 zeta chain, FcyRIII, FcsRI, a cytoplasmic tail of a Fc receptor and an immunoreceptor tyrosine-based activation motif (ITAM) bearing cytoplasmic receptors.

The term “chimeric antigen receptor” (“CAR” or “CARs”), as used herein, refers to engineered receptors which can confer an antigen specificity onto cells (for example, T cells such as naive T cells, central memory T cells, effector memory T cells or combinations thereof). CARs are also known as artificial T cell receptors, chimeric T cell receptors or chimeric immunoreceptors.

The antigen-specific targeting domain provides the CAR with the ability to bind to the target antigen of interest. The antigen-specific targeting domain preferably targets an antigen of clinical interest against which it would be desirable to trigger an effector immune response.

The antigen-specific targeting domain may be any protein or peptide that possesses the ability to specifically recognise and bind to a biological molecule (e.g. a hepatitis virus antigen). The antigen-specific targeting domain includes any naturally occurring, synthetic, semi-synthetic or recombinantly produced binding partner for a biological molecule of interest.

Illustrative antigen-specific targeting domains include antibody fragments or derivatives, extracellular domains of receptors, ligands for cell surface molecules/receptors, or receptor binding domains thereof.

In preferred embodiments, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) and a camelid antibody (VHH).

In preferred embodiments, the binding domain is a single chain antibody (scFv). The scFv may be, for example, a murine, human or humanised scFv.

The term “complementarity determining region” (“CDR”) with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs.

“Heavy chain variable region” (“VH”) refers to the fragment of the heavy chain of an antibody that contains three CDRs interposed between flanking stretches known as framework regions, which are more highly conserved than the CDRs and form a scaffold to support the CDRs.

“Light chain variable region” (“VL”) refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain.

“Single-chain Fv antibody” (“scFv”) refers to an engineered antibody consisting of a light chain variable region and a heavy chain variable region connected to one another directly or via a peptide linker sequence.

Antibodies that specifically bind a target antigen can be prepared using methods well known in the art. Such methods include phage display, methods to generate human or humanised antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

The CAR used in the present invention may also comprise one or more co-stimulatory domains. This domain may enhance cell proliferation, cell survival and development of memory cells.

Each co-stimulatory domain comprises the co-stimulatory domain of any one or more of, for example, members of the TNFR super family, CD28, CD137 (4-1BB), CD134 (OX40), DapIO, CD27, CD2, CD5, ICAM-1, LFA-1, Lck, TNFR-1, TNFR-II, Fas, CD30, CD40 or combinations thereof. Co-stimulatory domains from other proteins may also be used with the CAR used in the present invention.

The CAR used in the present invention may also comprise an intracellular signaling domain. This domain may be cytoplasmic and may transduce the effector function signal and direct the cell to perform its specialised function. Examples of intracellular signaling domains include, but are not limited to, ζ chain of the T cell receptor or any of its homologues (e.g. η chain, FcεR1γ and β chains, MB1 (Igα) chain, B29 (Igβ) chain, etc.), CD3 polypeptides (Δ, δ and ε), syk family tyrosine kinases (Syk, ZAP 70, etc.), src family tyrosine kinases (Lck, Fyn,

Lyn, etc.) and other molecules involved in T cell transduction, such as CD2, CD5 and CD28. The intracellular signaling domain may be human CD3 zeta chain, FcyRIII, FcsRI, cytoplasmic tails of Fc receptors, immunoreceptor tyrosine-based activation motif (ITAM) bearing cytoplasmic receptors or combinations thereof.

The CAR used in the present invention may also comprise a transmembrane domain. The transmembrane domain may comprise the transmembrane sequence from any protein which has a transmembrane domain, including any of the type I, type II or type III transmembrane proteins. The transmembrane domain of the CAR used in the present invention may also comprise an artificial hydrophobic sequence. The transmembrane domains of the CARs used in the present invention may be selected so as not to dimerise. Examples of transmembrane (TM) regions used in CAR constructs are: 1) The CD28 TM region (Pule et al, Mol Ther, 2005, November; 12(5):933-41; Brentjens et al, CCR, 2007, Sep. 15; 13(18 Pt 1):5426-Casucci et al, Blood, 2013, Nov. 14; 122(20):3461-72.); 2) The OX40 TM region (Pule et al, Mol Ther, 2005, November; 12(5):933-41); 3) The 41BB TM region (Brentjens et al, CCR, 2007, Sep. 15; 13(18 Pt 1):5426-35); 4) The CD3 zeta TM region (Pule et al, Mol Ther, 2005, November; 12(5):933-41; Savoldo B, Blood, 2009, Jun. 18; 113(25):6392-402.); 5) The CD8a TM region (Maher et al, Nat Biotechnol, 2002, January; 20(1):70-5.; Imai C, Leukemia, 2004, April; 18(4):676-84; Brentjens et al, CCR, 2007, Sep. 15; 13(18 Pt 1):5426-35; Milone et al, Mol Ther, 2009, August; 17(8): 1453-64.).

### Variants, Derivatives, Analogues, Homologues and Fragments

In addition to the specific proteins and nucleotides mentioned herein, the invention also encompasses variants, derivatives, analogues, homologues and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 96% or 97% or 98% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

### Method of Treatment

All references herein to treatment include curative, palliative and prophylactic treatment. The treatment of mammals, particularly humans, is preferred. Both human and veterinary treatments are within the scope of the invention.

In some embodiments, the method of treatment provides the agent and/or interleukin to the liver of a subject.

In some embodiments, the method of treatment provides the agent and/or interelukin to hepatocytes.

### Pharmaceutical Compositions and Injected Solutions

Although the agents for use in the invention can be administered alone, they will generally be administered in admixture with a pharmaceutical carrier, excipient or diluent, particularly for human therapy.

The medicaments, for example vectors or cells, of the invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used. In some cases, serum albumin may be used in the composition.

For injection, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

### Administration

In some embodiments, the agent and/or interleukin is administered to a subject locally.

In preferred embodiments, the agent and/or interleukin is administered to a subject's liver.

In some embodiments, the vector, cell or composition is administered to a subject locally.

In preferred embodiments, the vector, cell or composition is administered to a subject's liver.

The term “systemic delivery” or “systemic administration” as used herein means that the agent of the invention is administered into the circulatory system, for example to achieve broad distribution of the agent. In contrast, topical or local administration restricts the delivery of the agent to a localised area, e.g. the liver.

In some embodiments, the agent and/or interleukin is administered simultaneously, sequentially or separately in combination with a population of T cells. In some embodiments, the T cells express a chimeric antigen receptor (CAR) or a T cell receptor (TCR), optionally which binds to a hepatitis virus antigen.

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

### Dosage

The skilled person can readily determine an appropriate dose of an agent of the invention to administer to a subject. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is human.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

### Results and Discussion

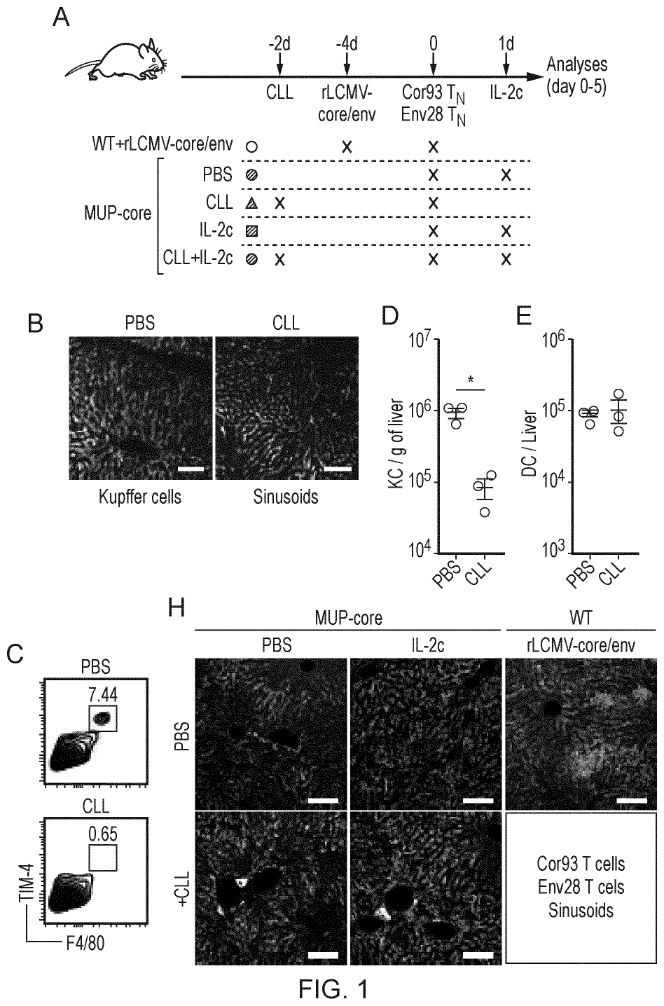

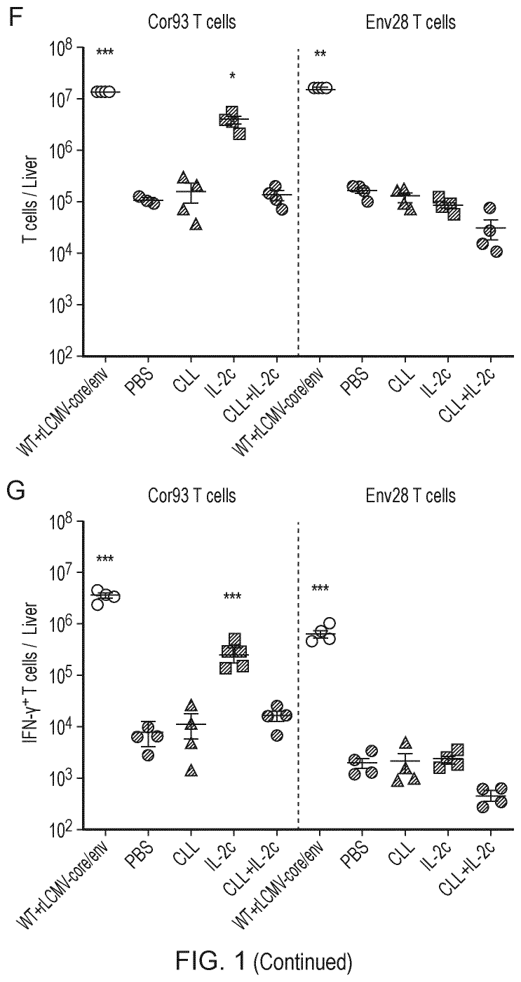

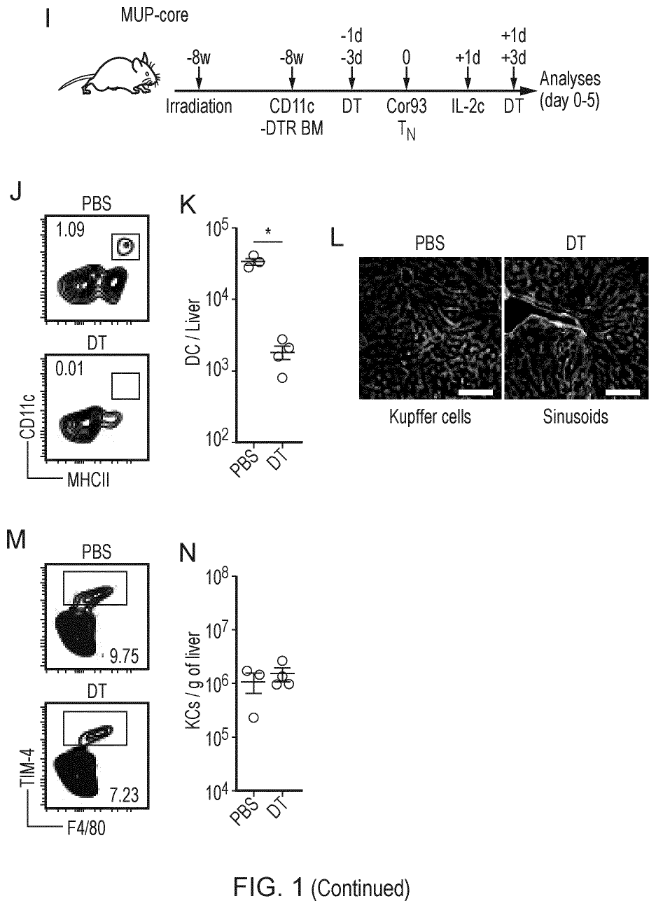

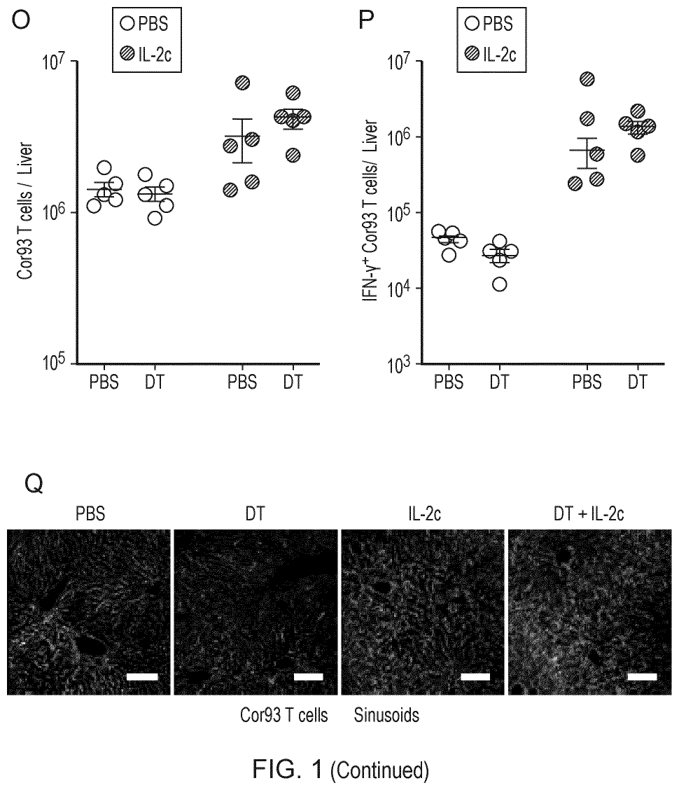

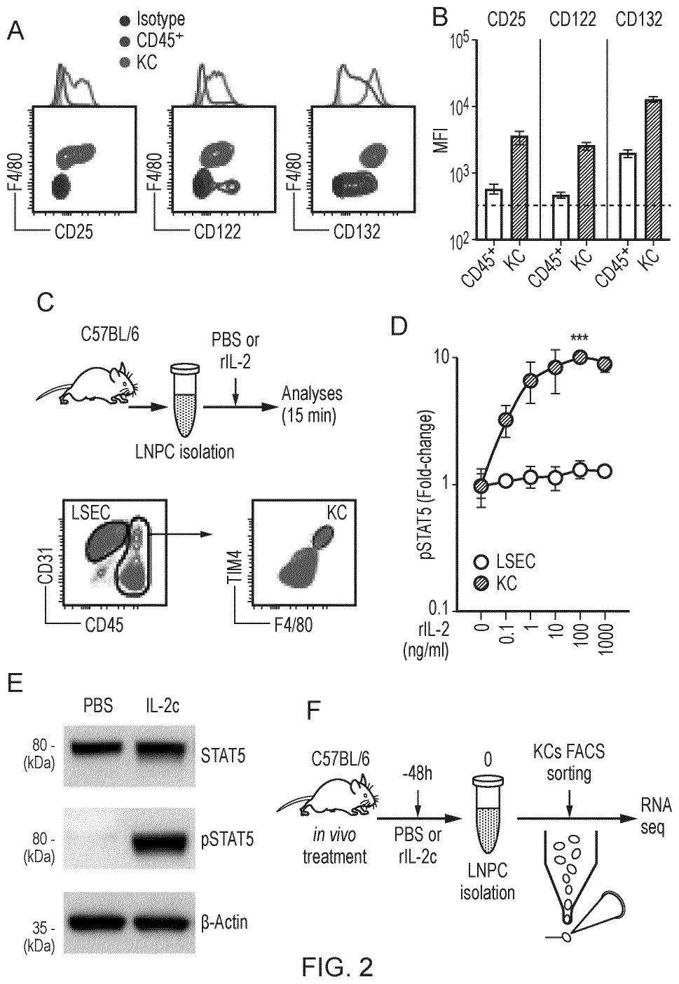

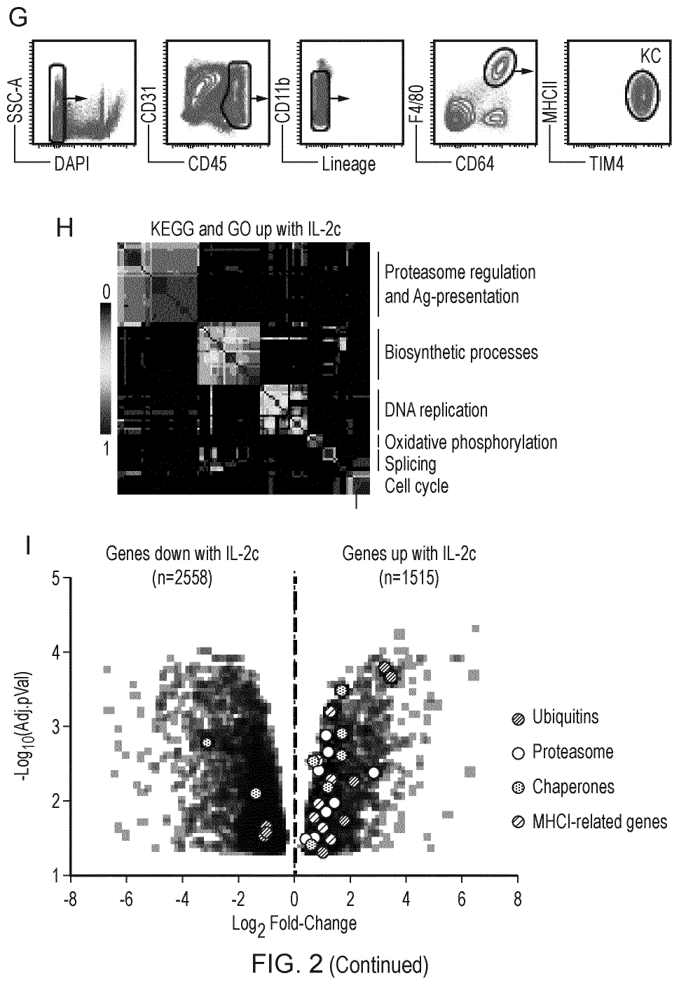

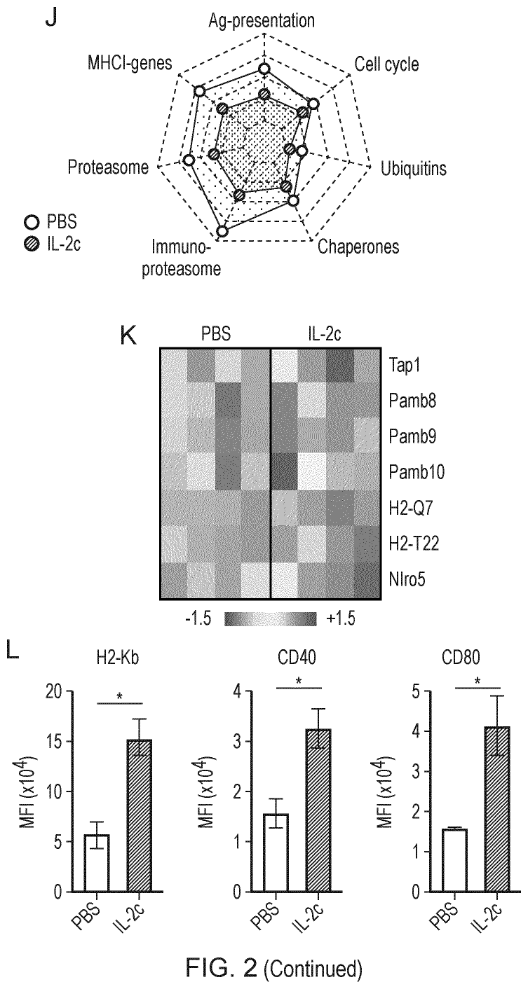

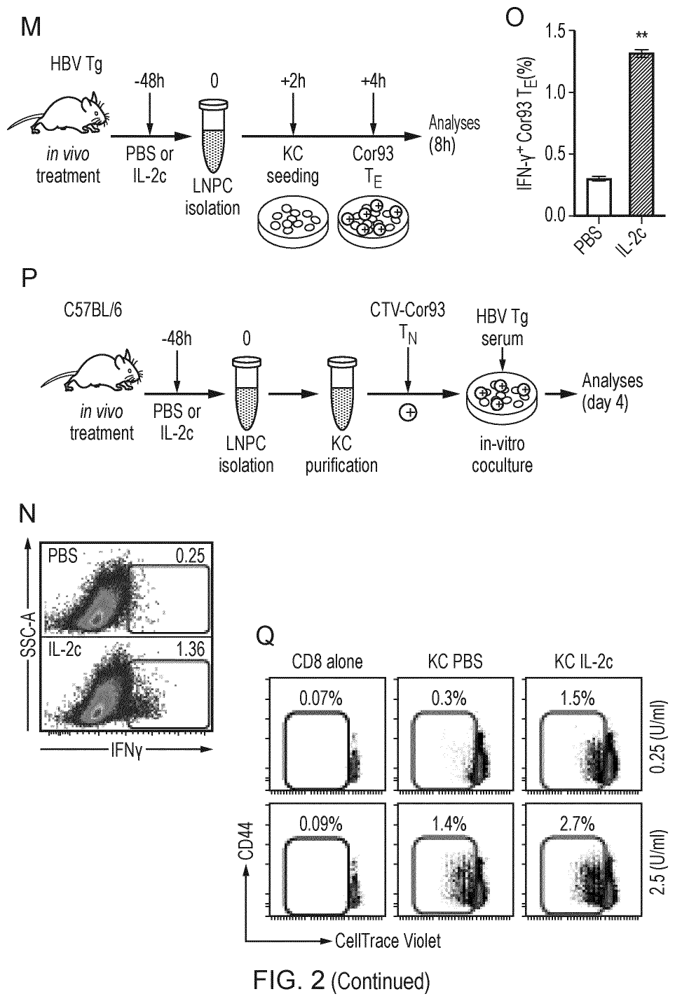

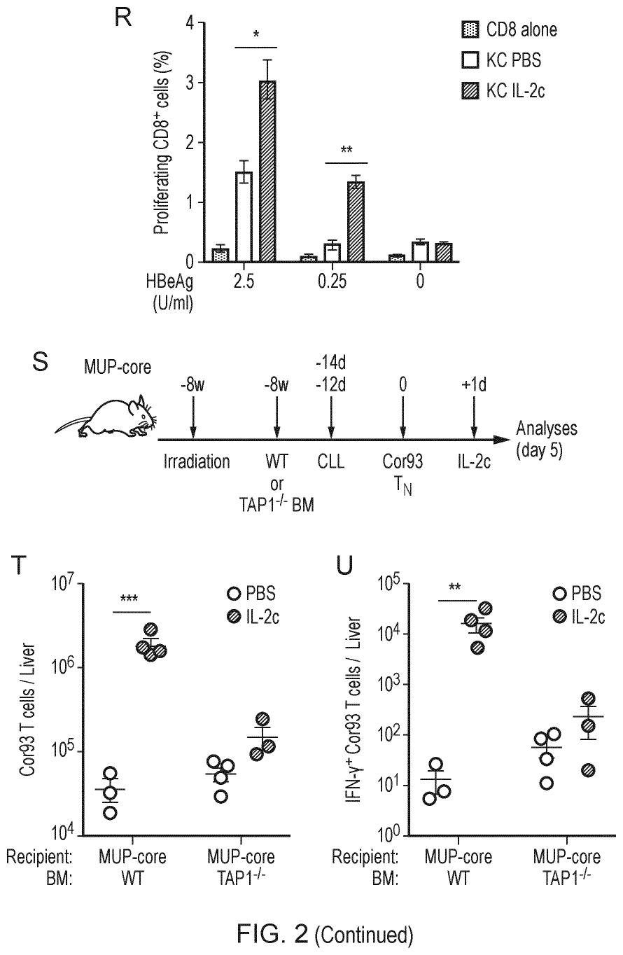

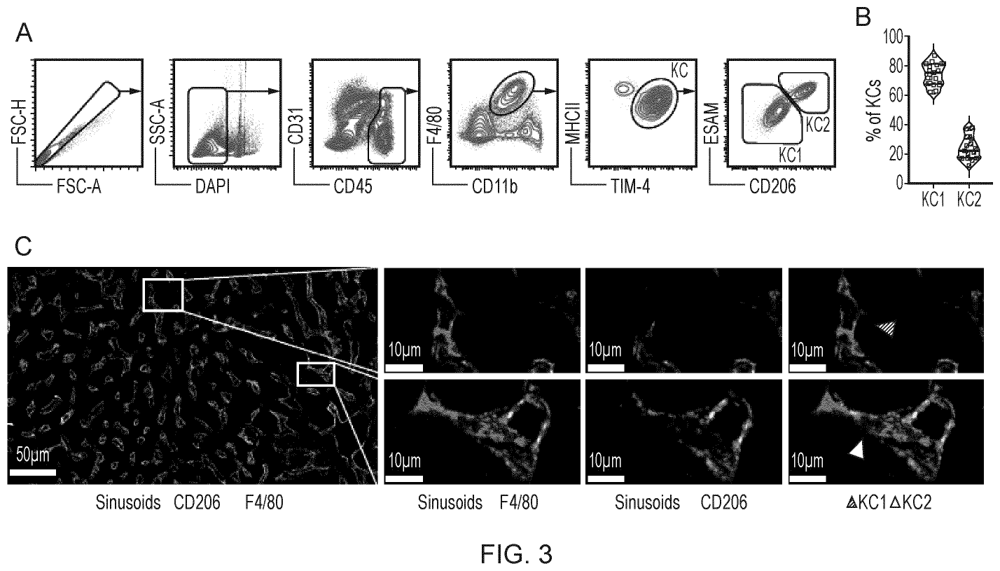

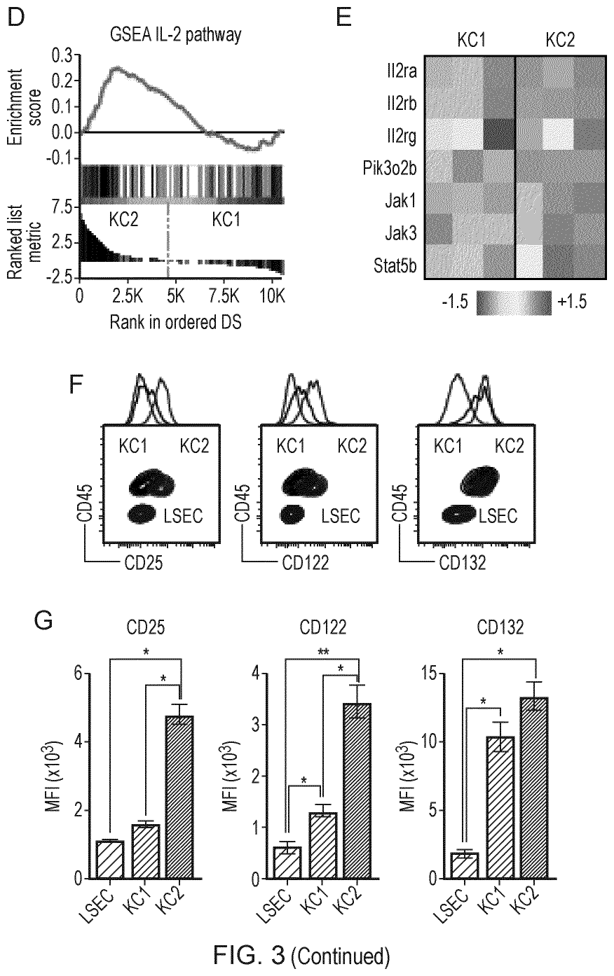

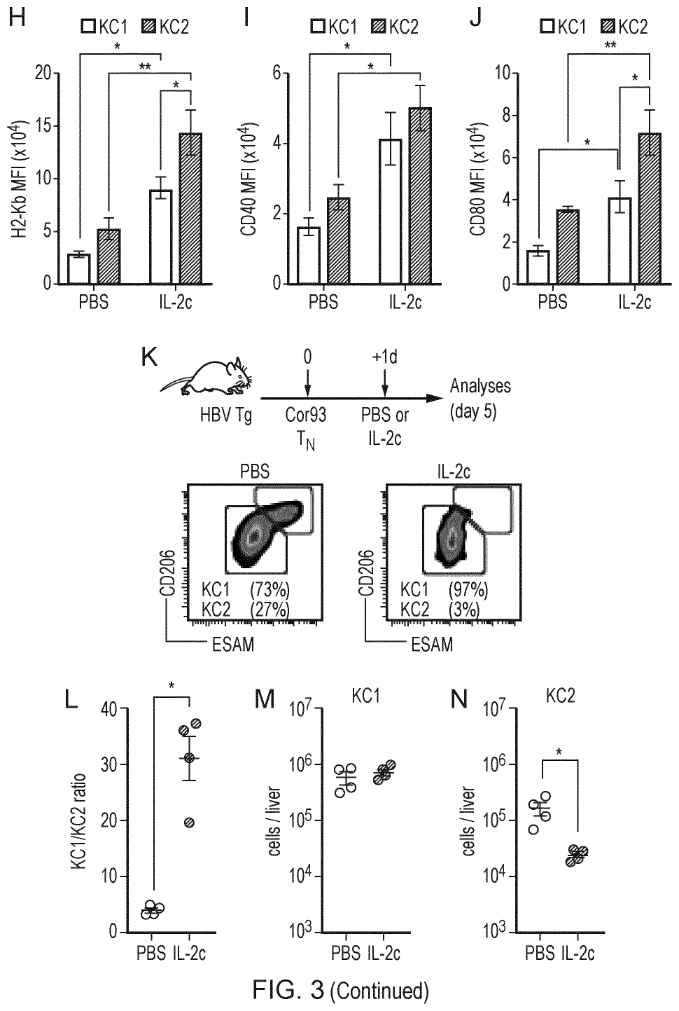

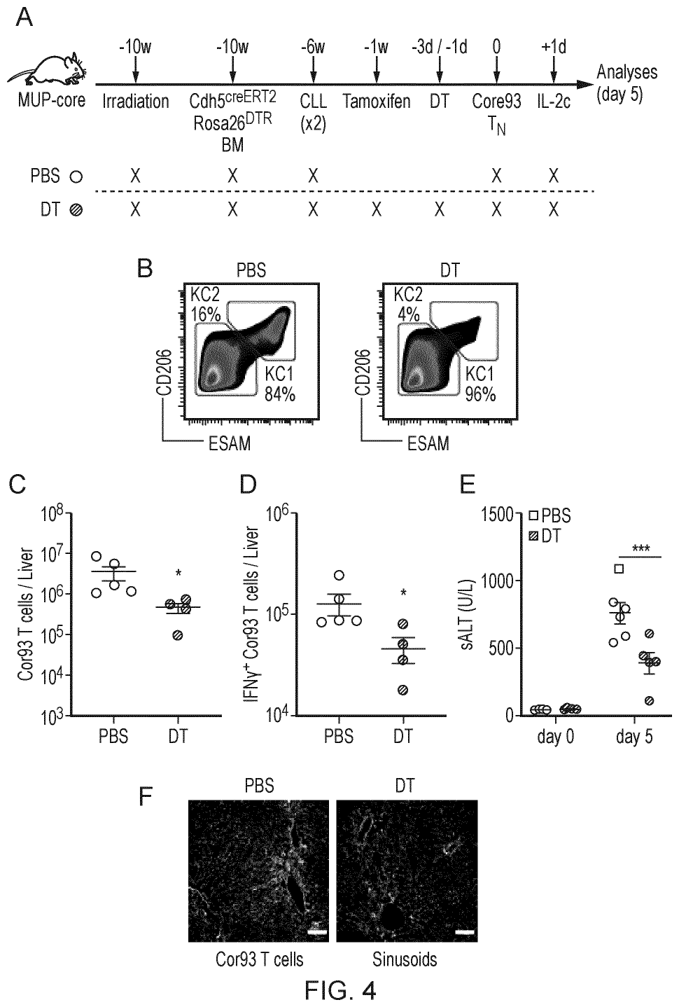

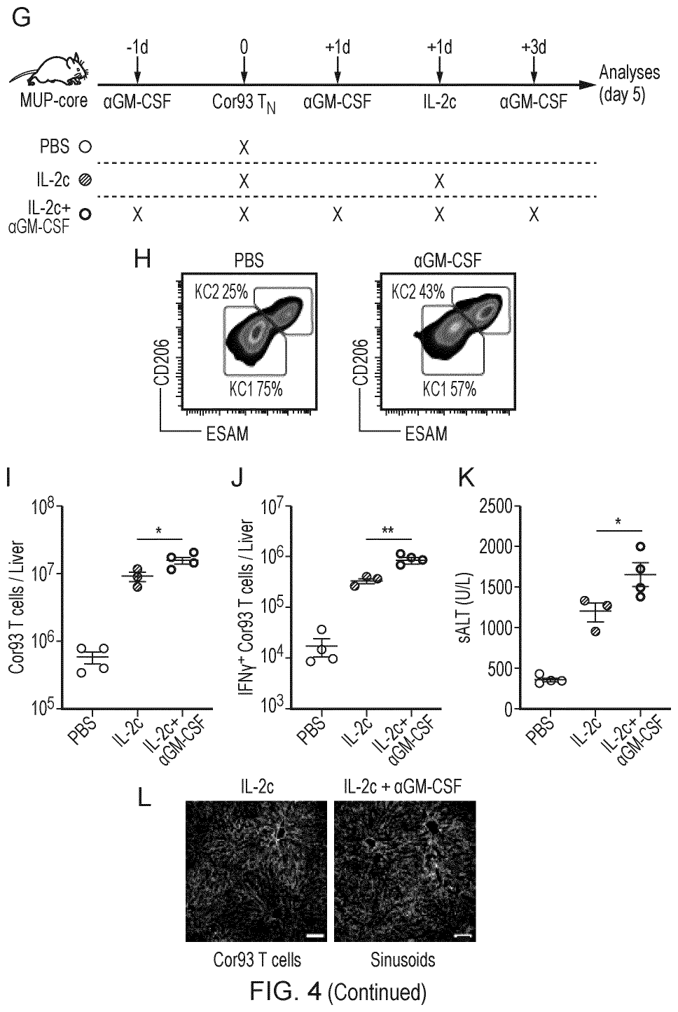

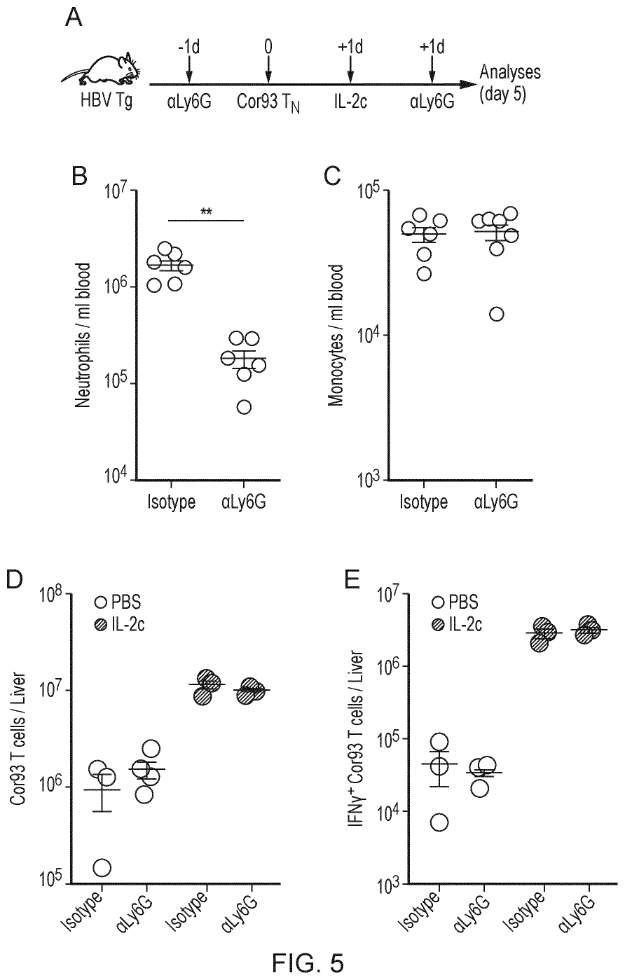

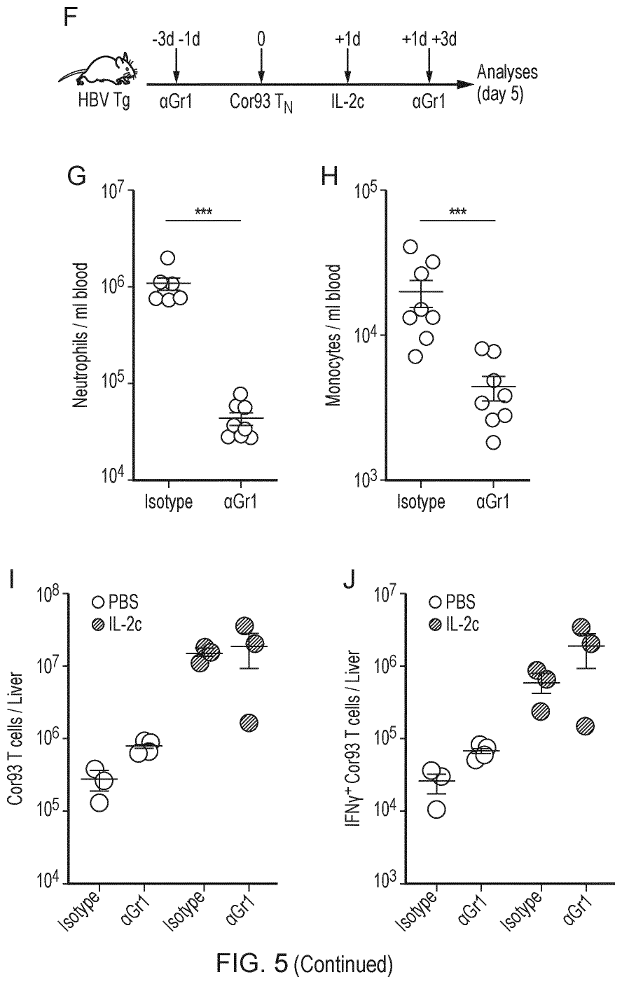

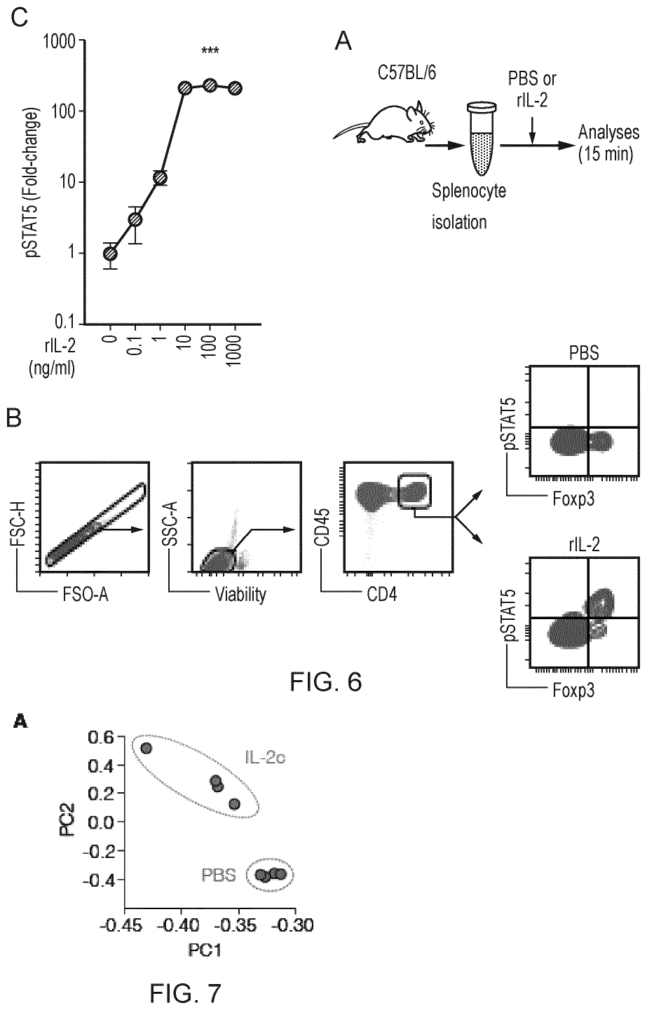

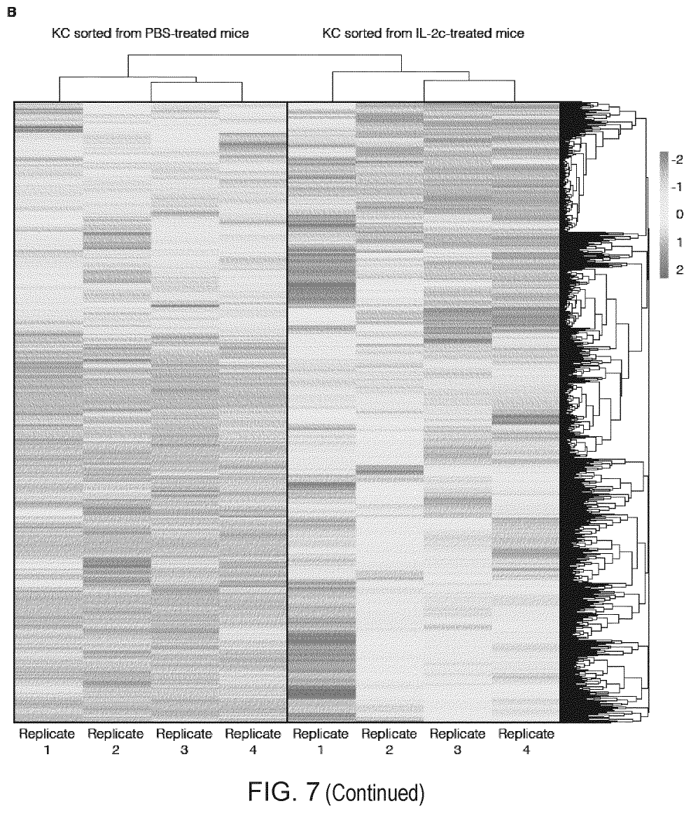

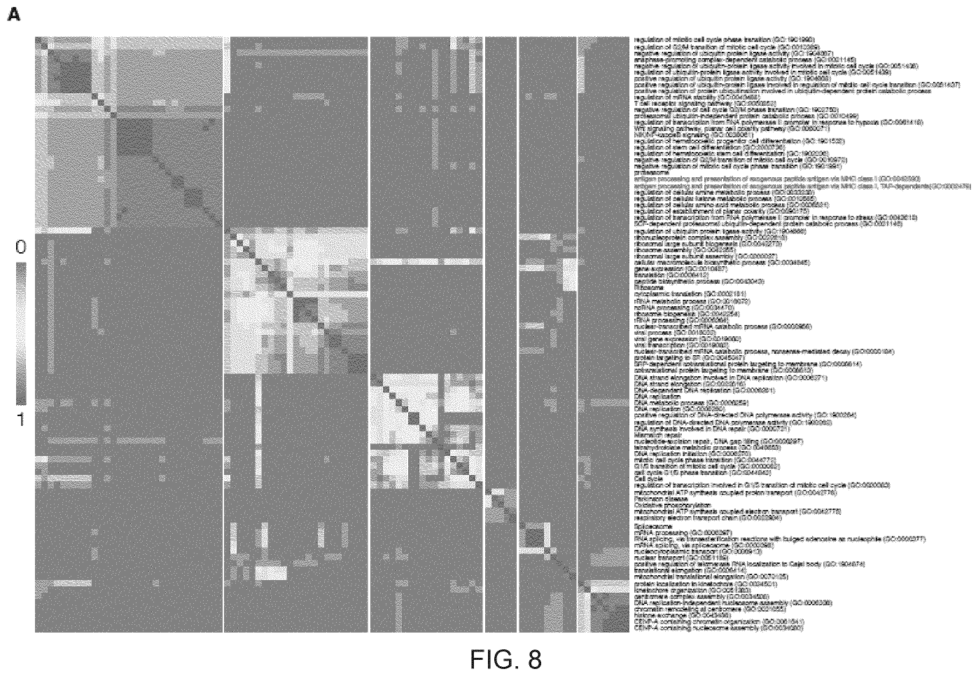

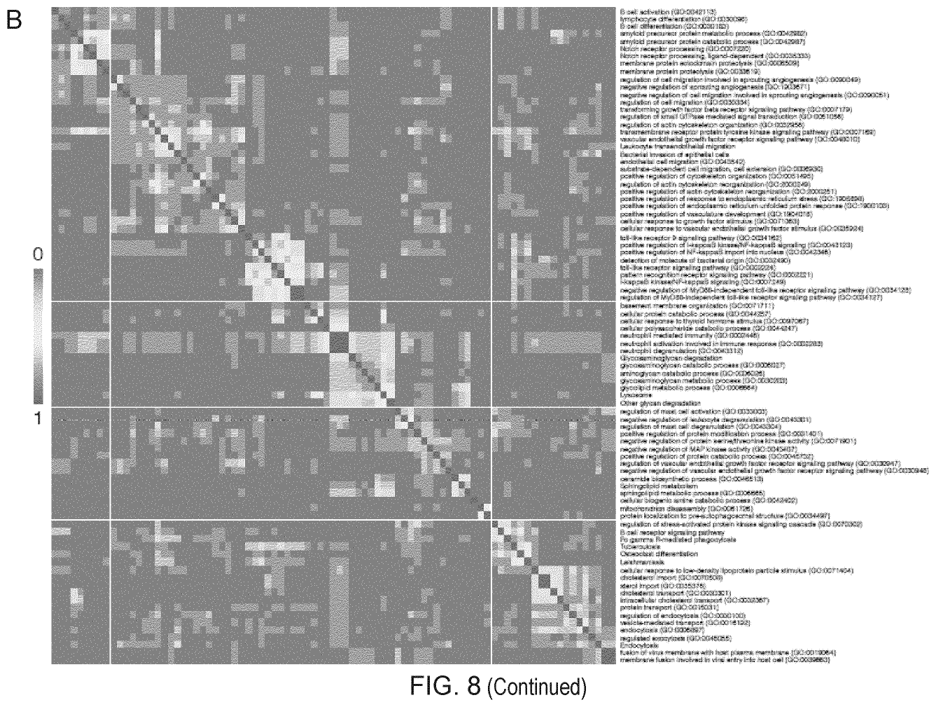

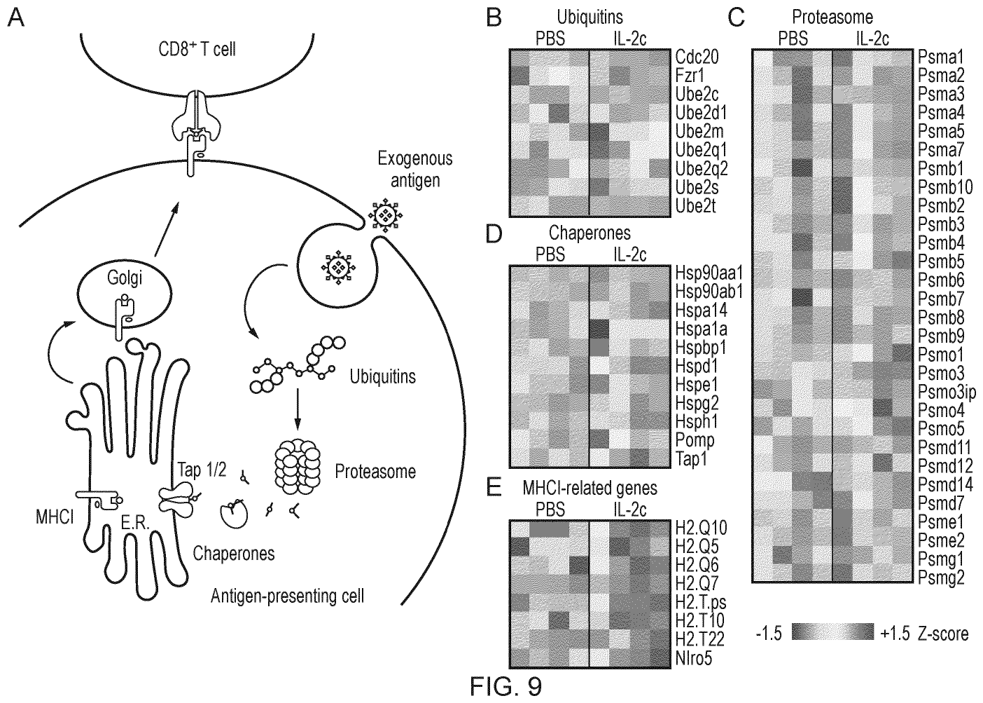

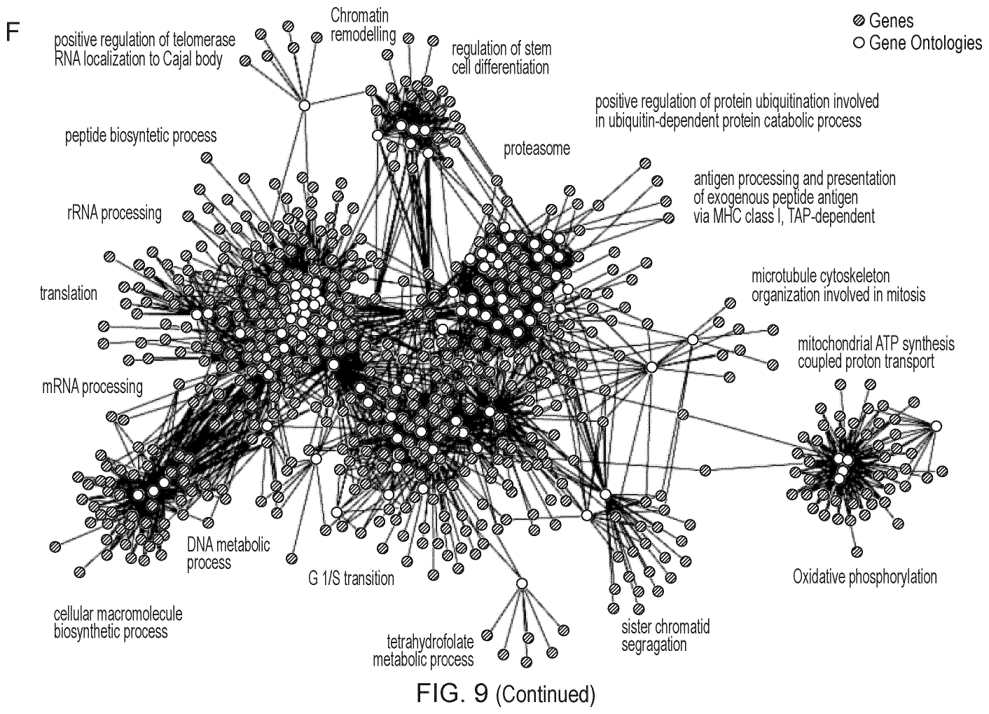

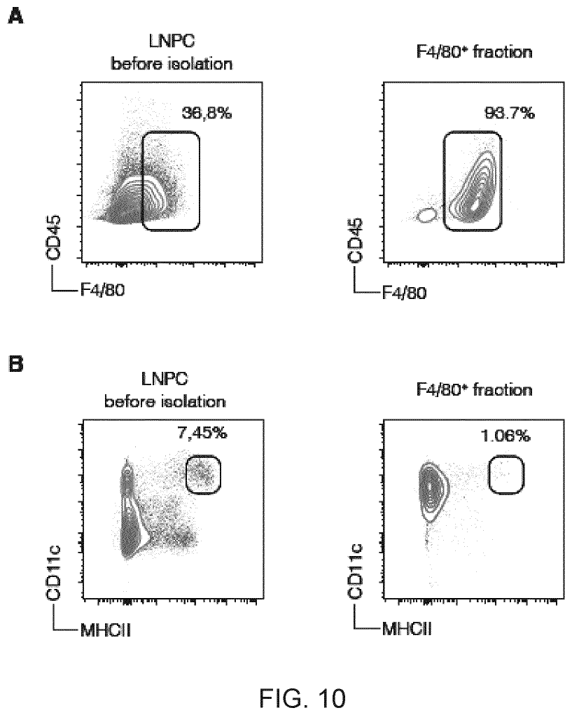

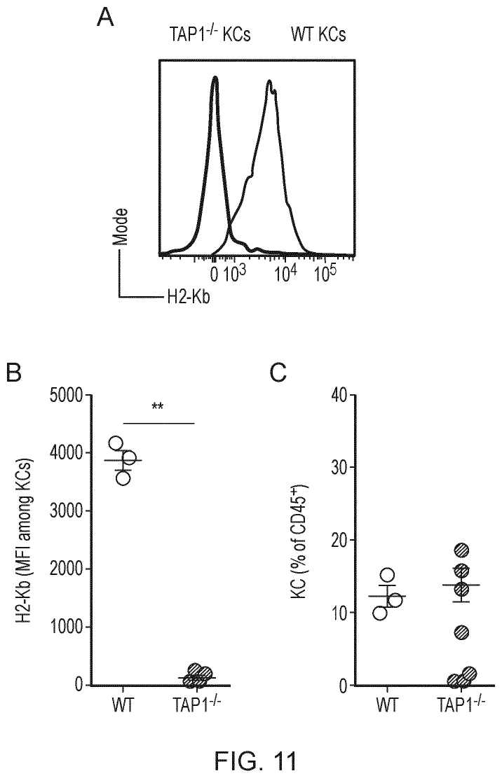

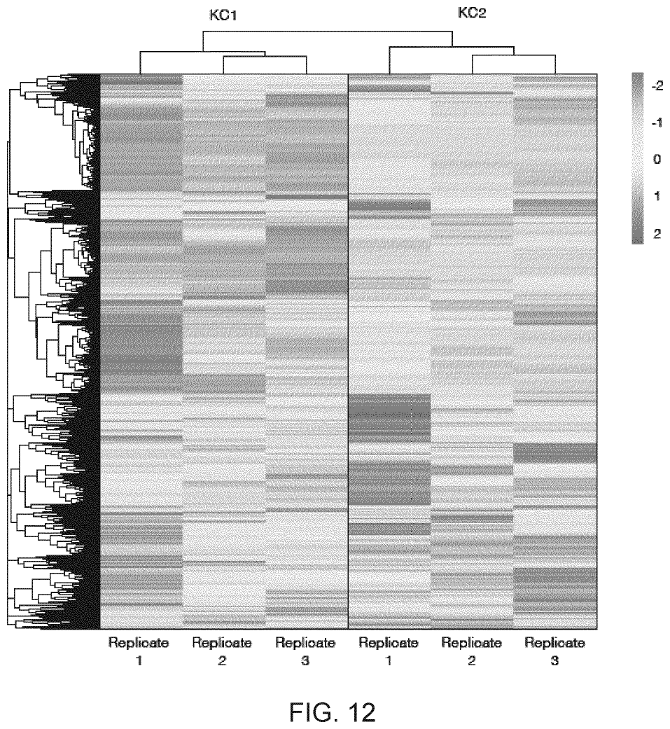

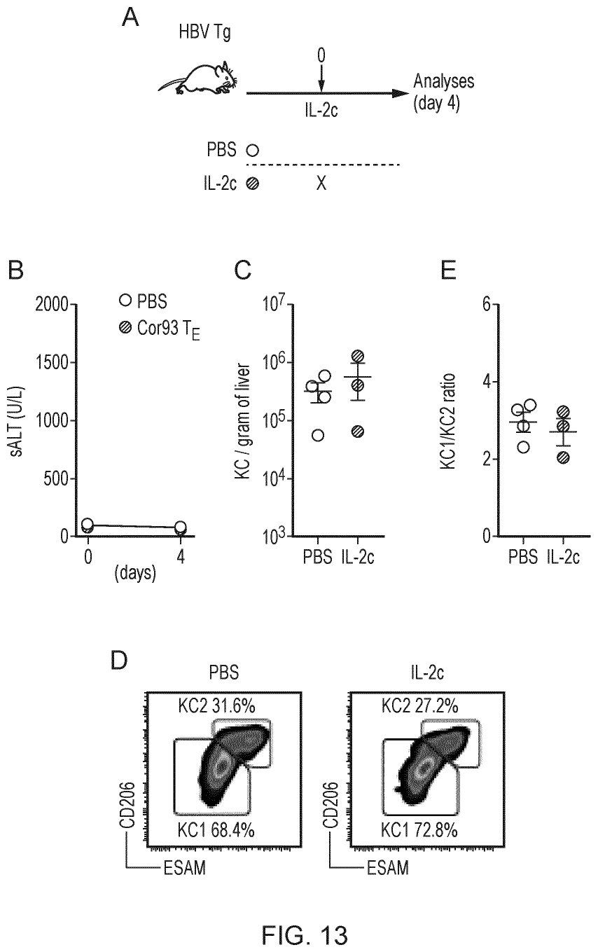

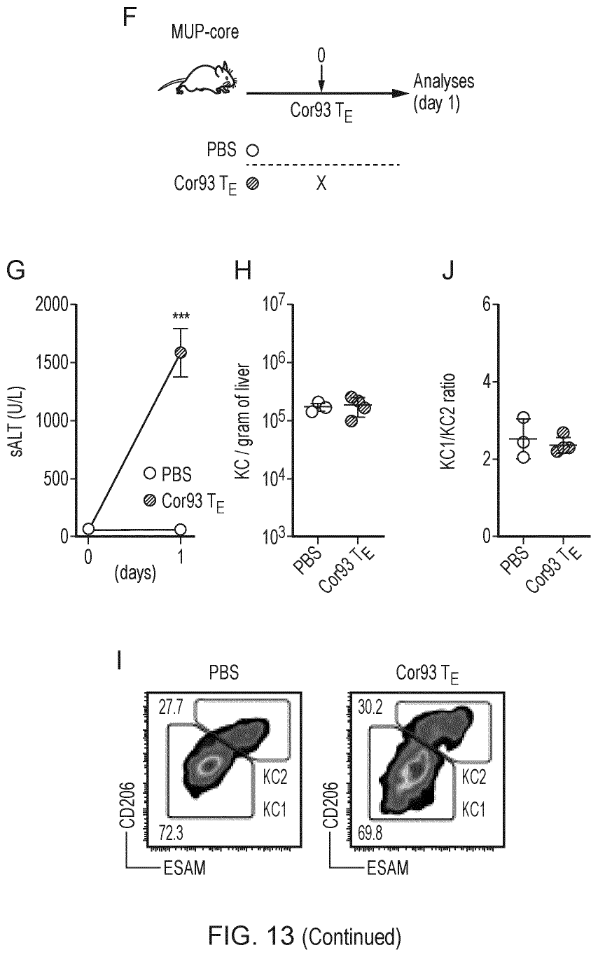

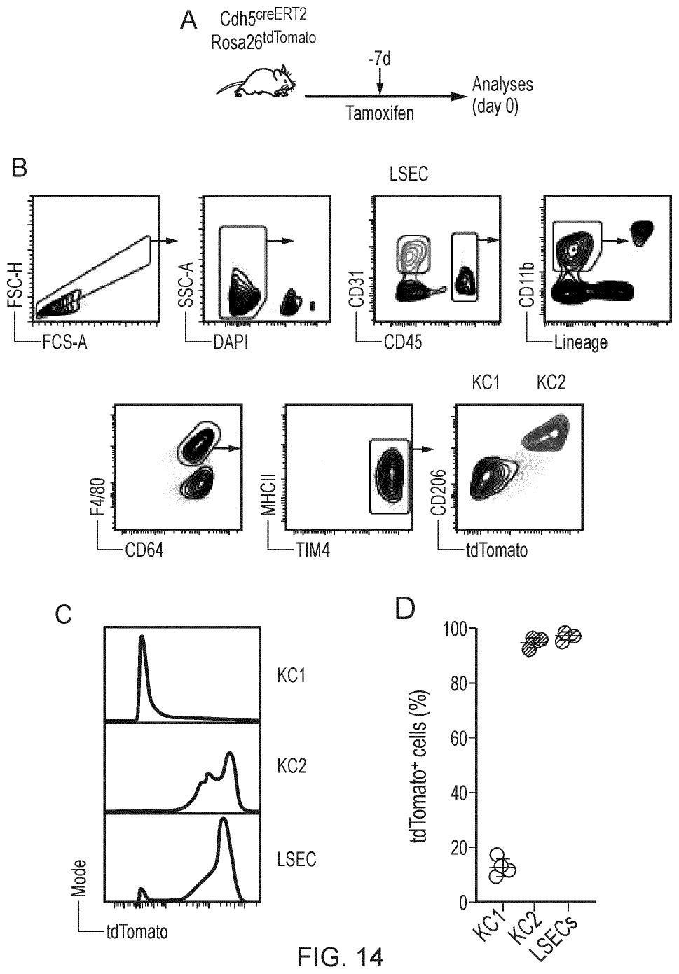

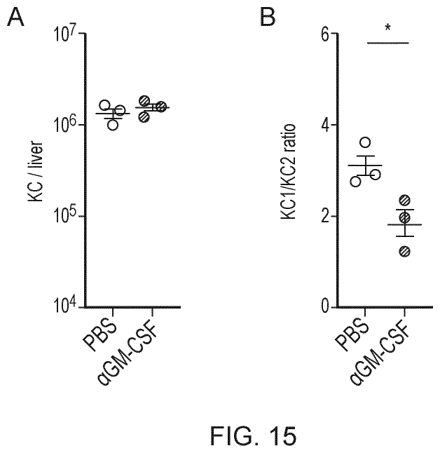

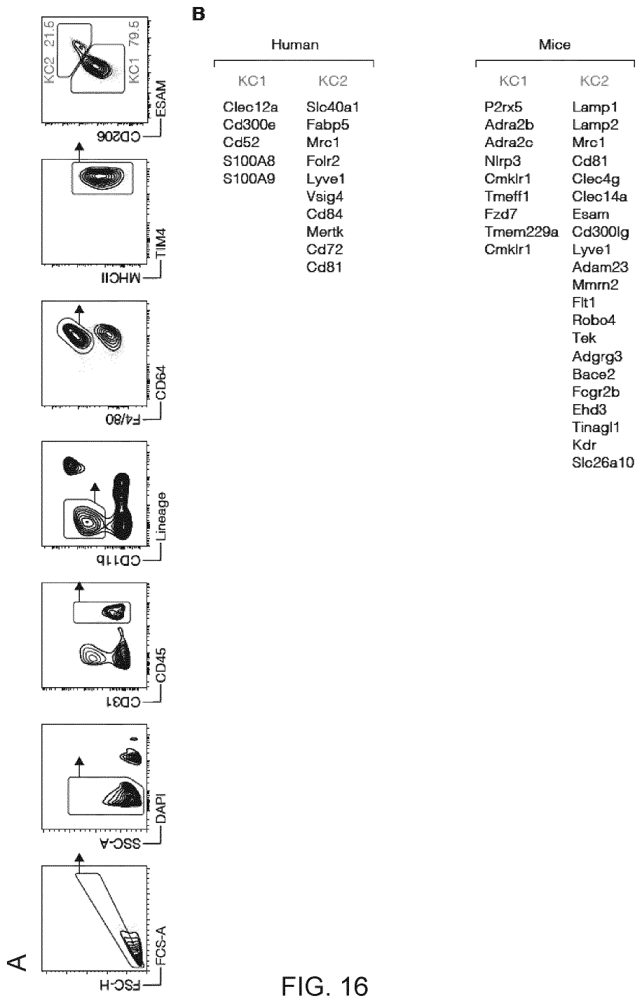

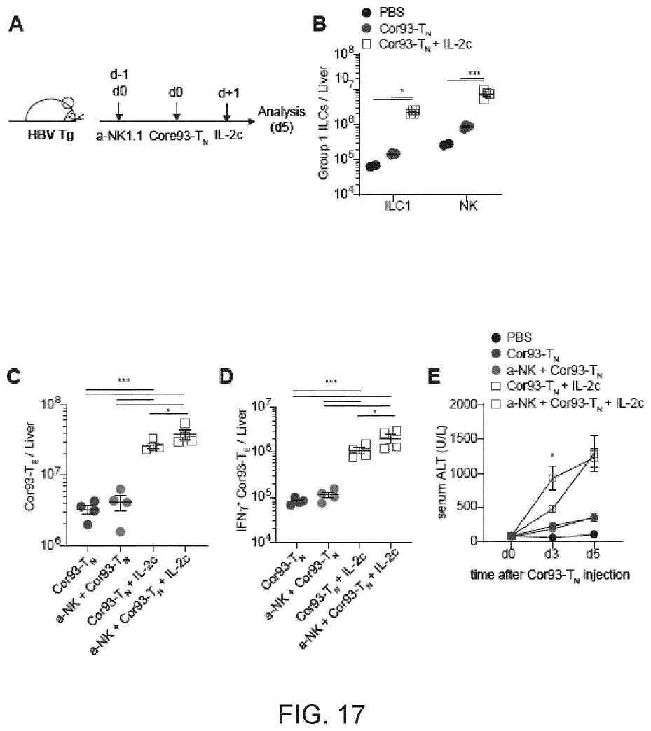

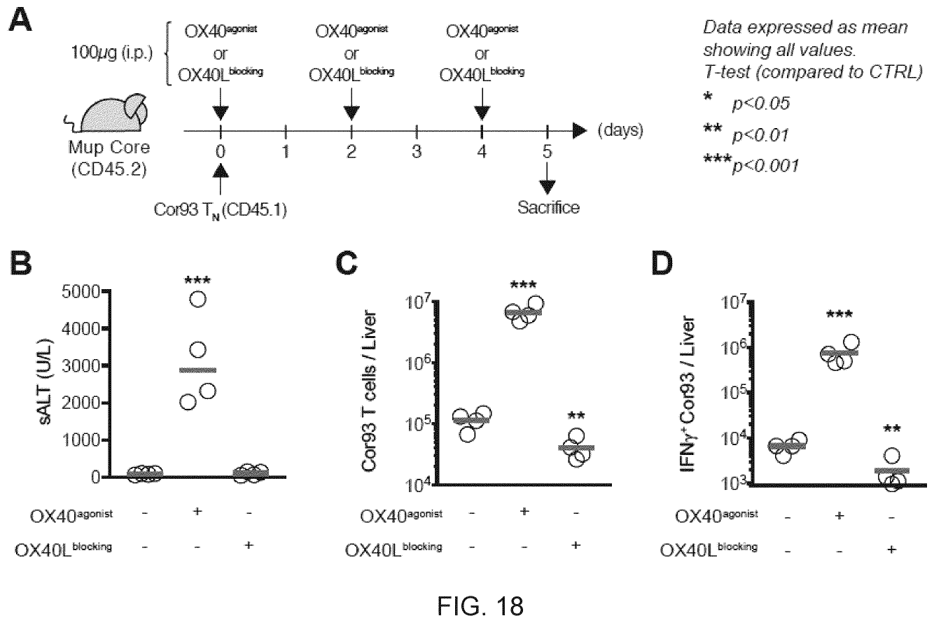

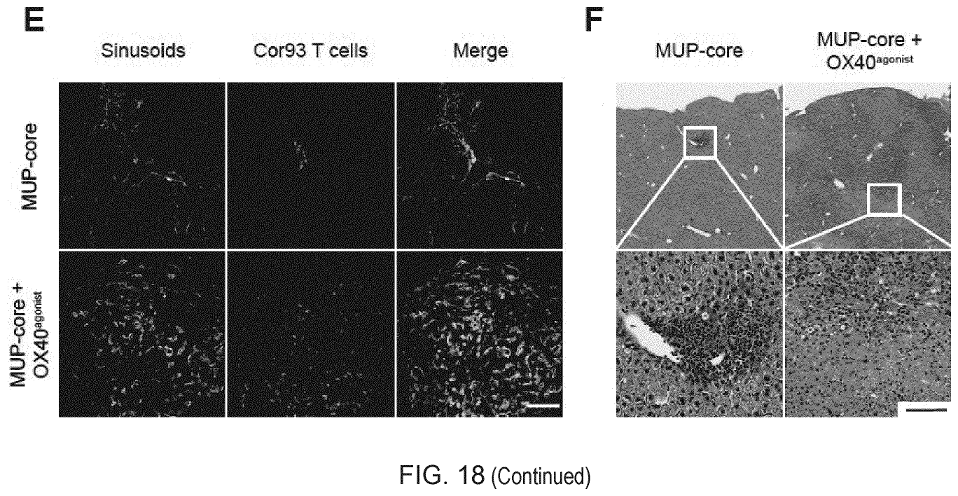

To shed light on the immune mechanisms underpinning the IL-2-mediated reinvigoration of intrahepatically-primed T cells, we initially took advantage of MUP-core transgenic mice that exclusively express a non-secretable version of the particulate HBV core protein in 100% of hepatocytes (L. G. Guidotti, V. Martinez, Y. T. Loh, C. E. Rogler, F. V. Chisari, Hepatitis B virus nucleocapsid particles do not cross the hepatocyte nuclear membrane in transgenic mice. Journal of Virology. 68, 5469-5475 (1994). These animals, like the HBV replication-competent transgenic mice described below, never develop spontaneous liver pathology as the hepatocellular expression of the viral gene products occurs non-cytopathically and endogenous T cells specific for these products are profoundly tolerant. As controls for proper CD8⁺ T cell differentiation into effector cells, we used WT mice transduced with recombinant, replication-defective lymphocytic choriomeningitis (LCMV)-based vectors targeting the HBV core and envelope proteins (rLCMV-core/env) to intrahepatic professional Ag-presenting cells (i.e. Kupffer cells (KCs) and hepatic DCs) that are not natural targets of HBV. Both groups of mice were injected with naïve CD8⁺ TCR transgenic T cells specific for epitopes contained within the core and envelope proteins of HBV (Cor93 and Env28 T_{N}, respectively) (FIG. 1A) (M. Isogawa, J. Chung, Y. Murata, K. Kakimi, F. V. Chisari, CD40 activation rescues antiviral CD8⁺ T cells from PD-1-mediated exhaustion. PLoS Pathogens. 9, e1003490 (2013)). One day after T_{N} injection, selected MUP-core mice received IL-2 immune complexes (IL-2c)—consisting of IL-2 coupled with non-neutralizing IL-2-specific monoclonal antibodies (S4B6) that enhance the half-life of IL-2 in vivo (FIG. 1A).

To test whether IL-2c treatment had exclusively a direct effect on T_{N} or whether it required the presence of additional cells, we performed depletion experiments. We initially focused on KCs, as these cells are capable of inducing full effector differentiation of CD8⁺ T cells upon in vivo rLCMV transduction. KCs were depleted through clodronate liposomes (CLL) injection two days prior to T cell injection (FIG. 1A). This treatment effectively depletes KCs while sparing hepatic DCs (FIG. 1B-E). Consistent with previously published results, Cor93 and Env28 T_{N} transferred to WT mice injected with rLCMV-core/env differentiated into bona fide effector cells that formed tight clusters scattered throughout the liver lobules; by contrast, Cor93 T cells transferred to MUP-core mice generated dysfunctional cells devoid of IFN-γ-producing ability that coalesced around portal tracts (FIG. 1F-H). IL-2c administration improved the capacity of Ag-specific Cor93 T cells to expand, differentiate into IFN-γ-producing cells and accumulate in clusters scattered throughout the liver lobules, but it had no effect on irrelevant Env28 T_{N} (FIG. 1F-H). Optimal in vivo reinvigoration of intrahepatically primed Cor93 T cells required the presence of KCs, as IL-2c treatment failed to improve T cell expansion, effector differentiation and intraparenchymal cluster formation in CLL-treated mice (FIG. 1F-H). Similar results were obtained when recombinant IL-2 was used in place of IL-2c and when HBV replication-competent transgenic mice—which express all viral proteins in hepatocytes and secrete enveloped virions containing the HBV particulate core protein into the bloodstream—were used in place of MUP-core recipients.

To confirm that hepatic DCs are not necessary for the optimal in vivo response to IL-2, we depleted this cell population by virtue of diphtheria toxin (DT) injection in MUP-core mice reconstituted with CD11c^{DTR} bone marrow (FIG. 1I). This treatment significantly decreased the number of hepatic DCs while sparing KCs (FIG. 1J-N). DC depletion did not affect the capacity of IL-2 to promote expansion, effector differentiation and intraparenchymal cluster accumulation of intrahepatically-primed Cor93 T cells (FIG. 1O-Q). Similarly, other phagocytic cells such as neutrophils and monocytes were found not to be involved in the response to IL-2 as neutrophil depletion (via anti-Ly6G Abs) or combined neutrophil and monocyte depletion (via anti-Gr1 Abs) did not affect the in vivo reinvigoration of intrahepatically-primed T cells by IL-2 (FIG. 5). Taken together, these results indicate that KCs are required for optimal in vivo reinvigoration of intrahepatically-primed T cells by IL-2.

Flow cytometric analyses revealed that a fraction of KCs express all 3 subunits of the IL-2 receptor (CD25, CD122 and CD132) (FIG. 2A, B). We therefore investigated the effect of IL-2 treatment on these cells. To this end, we isolated liver non parenchymal cells (LNPCs)—including KCs—from C57BL/6 mice and stimulated them ex vivo with recombinant IL-2 (FIG. 2C). We observed a dose-dependent increase in STAT5 phosphorylation in KCs, but not in liver sinusoidal endothelial cells (LSECs) (FIG. 2D). Similar results were obtained when IL-2c was used in place of IL-2, and STAT5 phosphorylation in KCs was confirmed by Western blot analysis (FIG. 2E). Of note, the IL-2-dependent fold change in STAT5 phosphorylation observed in KCs was ˜10-fold lower than that observed in CD4⁺ FoxP3⁺ splenic T regulatory cells (FIG. 6). Nevertheless, these data indicate that KCs express a functional IL-2 receptor capable of responding to IL-2 in vitro.

To assess the consequences of IL-2 treatment on KCs in vivo, we treated C57BL/6 mice with IL-2c and then performed RNA-seq analysis on FACS-sorted KCs 48 hours later (FIG. 2F, G). 4073 Differentially Expressed Genes (DEGs)—1515 up- and 2558 down-regulated—were identified as significantly regulated by IL-2c (FIG. 7). Functional enrichment analysis of up-regulated genes showed an increased transcription of genes involved mainly in antigen presentation and proteasomal processing, ribosomal RNA processing and splicing, DNA replication and cell cycle, as well as mitochondrial oxidative metabolism (FIG. 2H, FIG. 8, 9). Among the up-regulated gene clusters, we focused on the antigen presentation pathway which includes several macromolecular complexes comprised of ubiquitins, chaperones, MHC-I and proteasome subunits (FIG. 9A). Genes encoding for these protein families—specifically MHC-I-related proteins, immunoproteasome subunits, the transcription regulator of MHC-I genes NIrc5 and the transporter associated with antigen processing 1 (Tap1)—were induced in KCs upon IL-2c treatment (FIG. 2I-K and FIG. 9B-F). The up-regulation of MHC-I and co-stimulatory molecules in KCs isolated from mice treated with IL-2c was confirmed at the protein level (FIG. 2L).

Based on these results, we reasoned that in vivo treatment with IL-2c might increase the cross-presentation ability of KCs. To test this possibility, we measured the capacity of in vitro differentiated Cor93-specific effector CD8⁺ T cells (Cor93 T_{E}) to produce IFN-γ (as an indirect measure of Ag recognition) upon incubation with KCs isolated from control and IL-2c-treated HBV replication-competent transgenic mice (FIG. 2M). Consistently with previously published data, baseline KC cross-presentation of the core protein in this experimental system at steady state is negligible (FIG. 2N, O), despite KCs being constantly exposed to abundant HBV virions in the circulation. Cor93 T_{N} remain dysfunctional even when isolated from the liver of HBV replication-competent transgenic mice previously transferred with highly pathogenic Env28-specific effector CD8⁺ T cells. This indicates that KC cross-presentation remains insignificant during acute liver inflammation, even though the inflammatory conditions potentially favor not only the uptake of HBV virions but also the phagocytosis of damaged hepatocytes containing the particulate HBV core protein. In spite of this, treating HBV replication-competent transgenic mice with IL-2c slightly but significantly increased the cross-presentation capacity of KCs incubated in vitro with Cor93 T_{E} (FIG. 2N, O).

We also assessed the ability of KCs isolated from IL-2-treated C57BL/6 mice (purity shown in FIG. 10) to cross-prime HBV-specific naïve CD8⁺ T cells exposed to the serum of HBV replication-competent transgenic mice in vitro (FIG. 2P). When compared to KCs isolated from PBS-treated mice, KCs exposed to IL-2 in vivo induced a higher proliferation of Cor93 T_{N} in in vitro culture (FIG. 2Q, R).

Finally, to evaluate the in vivo relevance of our findings, we took advantage of MUP-core mice which express only a non-secretable, particulate form of the HBV core protein and where KC cross-presentation should depend on the uptake of the few hepatocytes that are known to be injured by Cor93 T_{N} transfer. We generated MUP-core mice whose hematopoietic cells (including KCs) lack Transporter associated with Antigen Processing 1 (TAP-1) and therefore cannot express MHC-I and present Ags to CD8⁺ T cells (FIG. 2S). This was achieved by injection of either WT or TAP-1^{-/-} bone marrow into irradiated MUP-core mice, followed by CLL treatment to deplete the residual radio-resistant KCs and allow the complete reconstitution of the entire KC compartment with bone marrow-derived cells (G. Sitia, M. Iannacone, R. Aiolfi, M. Isogawa, N. van Rooijen, C. Scozzesi, M. E. Bianchi, U. H. von Andrian, F. V. Chisari, L. G. Guidotti, Kupffer cells hasten resolution of liver immunopathology in mouse models of viral hepatitis. PLoS Pathogens. 7, e1002061 (2011))(FIG. 11). Cor93 T_{N} injected into MUP-core mice whose hematopoietic cells (including KCs) lacked MHC-I (FIG. 11) had a much lower response to IL-2c than did Cor93 T_{N} injected into mice carrying Ag presentation-competent KCs (FIG. 2T, U), suggesting that Cor93 T cells interacted with IL-2-stimulated KCs that cross-presented core protein-derived epitopes after the uptake of damaged hepatocytes. Taken together, these results indicate that optimal reinvigoration of intrahepatically primed CD8⁺ T cells by IL-2 requires the capacity of KCs to cross-present HBV antigens, possibly derived from circulating virions and/or damaged hepatocytes.

Next, we asked whether IL-2 acts homogenously on all KCs or whether a specific KC subset is responsible for the observed effect, as only a fraction of KCs expresses all 3 subunits of the IL-2 receptor (FIG. 2A, B). To this end, we employed high-dimensional single-cell RNA-sequencing (scRNA-seq) and flow cytometry analyses to characterize Kupffer cells isolated from C57BLJ6 mice. Two distinct populations of KCs (referred to as KC1 and KC2) have been identified that can be distinguished using a number of markers such as CD206 and ESAM (FIG. 3A-C). KC2 were identified as CD206^{high} ESAM^{high} cells and represent ˜15-30% of total KCs (FIG. 3A, B). Imaging analyses confirmed the presence of two distinct KC subpopulations (FIG. 3C). Importantly, RNA-seq analyses on KC1 and KC2 sorted from C57BL/6 mice revealed that KC2 are enriched in IL-2 signaling components (IL-2 receptor subunits and molecules implicated in intracellular signal transduction) (FIG. 3D, E, FIG. 12). Higher expression of the IL-2 receptor subunits, MHC-I and co-stimulatory molecules in KC2 was confirmed at the protein level by FACS analysis (FIG. 3F-J). Together, the data suggest that KC2 are better equipped than other KC subsets to respond to IL-2 and increase their capacity to cross-present hepatocellular Ags. Thus, one might predict that IL-2 treatment might render KC2 more sensitive than KC1 to CD8⁺ T cell-mediated killing.

To test this hypothesis, we treated HBV replication competent transgenic mice with IL-2c 24 hours after Cor93 T_{N} injection and checked the KC1/KC2 ratio 4 days later (FIG. 3K). Consistent with the hypothesis that IL-2 preferentially increases the capacity of KC2 to cross-present hepatocellular Ags and thus renders them more sensitive to CD8⁺ T cell-mediated killing, we found that KC2 almost completely disappeared in Cor93 T cell-injected HBV transgenic mice treated with IL-2c (FIG. 3K-N). Notably, neither IL-2c treatment alone (in the absence of Cor93 T_{N} transfer) nor severe liver inflammation (induced by Cor93 T_{E}) altered the KC1/KC2 ratio (FIG. 13).

We next sought to generate a model where KC2 could be selectively depleted to assess their role in the cross-presentation of hepatocellular Ags upon in vivo IL-2 treatment. We took advantage of the observation that KC2 (but not KC1) express the endothelial cell marker VE-cadherin (encoded by Cdh5) (FIG. 14) to establish a system allowing inducible depletion of KC2 but not endothelial cells. This was achieved by injecting Cdh5^{CreERT2}; R26^{iDTR} bone marrow into irradiated MUP-core mice, depleting the residual radio-resistant KCs by CLL to allow the complete reconstitution of the entire KC compartment with bone marrow-derived cells, inducing DTR expression in KC2 by tamoxifen administration and, finally, depleting KC2 by DT injection prior to Cor93 T_{N} transfer followed by IL-2c treatment (FIG. 4A, B). DT treatment caused a ˜4-fold decrease in KC2 (FIG. 4B) and resulted in a lower ability of Cor93 T cells to proliferate and differentiate into cytotoxic effector cells clustered throughout the liver lobule in response to IL-2 (FIG. 4C-F). These data indicate that KC2 are required for the optimal reinvigoration of intrahepatically primed T cells by IL-2.

Finally, we sought to identify experimental settings that might skew the KC1/KC2 ratio. We focused on the cytokine GM-CSF that is known to regulate macrophage differentiation. Antibody-mediated blockade of the cytokine GM-CSF resulted in a ˜50% increase in the relative proportion of KC2 (FIG. 4G, H and FIG. 15). Combining anti-GM-CSF treatment to IL-2c administration resulted in a superior ability of IL-2 to promote proliferation and effector differentiation of intrahepatically primed T cells (FIG. 4I-L). The data suggest that strategies aimed at increasing the number of KC2 may potentiate the capacity of IL-2 to revert the T cell dysfunction induced by hepatocellular priming.

We have delineated the mechanisms by which hepatocellularly-primed HBV-specific CD8⁺ T cells acquire antiviral and pathogenic effector functions following the exogenous administration of IL-2. These mechanisms rely on a hitherto unidentified subset of KCs—referred to as KC2—that is poised to respond to IL-2 and cross-present viral Ags contained within circulating virions or within hepatocytes. These results are noteworthy considering that steady-state KC cross-presentation of HBV Ags is a remarkably inefficient process that cannot be increased by liver inflammation, hepatocellular death or by the administration of therapeutic monoclonal antibodies directed against HBsAg leading to the generation of circulating immune. Our data do not rule out a direct effect of IL-2 on T cells; however, they indicate that optimal in vivo reinvigoration of intrahepatically primed T cells by IL-2 depends on the presence of KC2.

### Materials and Methods

### Mice

C57BLJ6, CD45.1 (inbred C57BLJ6), Balb/c, Thy1.1 (CBy.PL(B6)-Thy^{a}/ScrJ), β-actin-GFP [C57BLJ6-Tg(CAG-EGFP)1Osb/J], Ai14(RCL-tdT)-D [B6.Cg-Gt(ROSA)26Sortm14(CAG-tdTomato)Hze/J], β-actin-DsRed [B6.Cg-Tg(CAGDsRed*MST)1Nagy/J], Tap1-deficient (B6.129S2-Tap1^{tm1Arp}/J), CD11c^{DTR} [B6.FVB-1700016L2Rik^{Tg(Itgac-DTR/EGFP)57Lan}/J], ROSA26^{iDTR} [C57BL/6-Gt(ROSA)26Sortm1(HBEGF)Awai/J], Cdh5^{CreERT2} [Tg(Cdh5-cre/ERT2)1Rha] mice were purchased from Charles River or The Jackson Laboratory. MUP-core transgenic mice (lineage MUP-core 50 [MC50], inbred C57BLJ6, H-2^{b}), that express the HBV core protein in 100% of the hepatocytes under the transcriptional control of the mouse major urinary protein (MUP) promoter, have been previously described (L. G. Guidotti, V. Martinez, Y. T. Loh, C. E. Rogler, F. V. Chisari, Hepatitis B virus nucleocapsid particles do not cross the hepatocyte nuclear membrane in transgenic mice. J Virol. 68, 5469-75 (1994)). HBV replication-competent transgenic mice (lineage 1.3.32, inbred C57BLJ6, H-2^{b}), that express all of the HBV antigens and replicate HBV in the liver at high levels without any evidence of cytopathology, have been previously described (L. G. Guidotti, B. Matzke, H. Schaller, F. V. Chisari, High-level hepatitis B virus replication in transgenic mice. Journal of Virology. 69, 6158-6169 (1995)). In indicated experiments, MUP-core and HBV replication-competent transgenic mice were used as C57BL/6×Balb/c H-2^{bxd} F1 hybrids. Cor93 TCR transgenic mice (lineage BC10.3, inbred CD45.1), in which >98% of the splenic CD8⁺ T cells recognize a Kb-restricted epitope located between residues 93-100 in the HBV core protein (MGLKFRQL), have been previously described (M. Isogawa, J. Chung, Y. Murata, K. Kakimi, F. V. Chisari, CD40 activation rescues antiviralCD8⁺ T cells from PD-1-mediated exhaustion. PLoS Pathogens. 9, e1003490 (2013)). Env28 TCR transgenic mice (lineage 6C2.36, inbred Thy1.1 Balb/c), in which ˜83% of the splenic CD8⁺ T cells recognize a L^{d}-restricted epitope located between residues 28-39 of HBsAg (IPQSLDSVWVTSL), have been previously described (Isogawae et al. 2013). For imaging experiments Cor93 transgenic mice were bred against β-actin-GFP, while Env28 transgenic mice were bred against β-actin-DsRed mice (inbred Balb/c). Bone marrow (BM) chimeras were generated by irradiation of MUP-core or C57BL/6 mice with one dose of 900 rad and reconstitution with the indicated BM; mice were allowed to reconstitute for at least 8 weeks before experimental manipulations. Mice were housed under specific pathogen-free conditions and entered experiments at 8-10 weeks of age. In all experiments, mice were matched for age, sex and (for the 1.3.32 animals) serum HBeAg levels before experimental manipulations. All experimental animal procedures were approved by the Institutional Animal Committee of the San Raffaele Scientific Institute and are compliant with all relevant ethical regulations.

### Viruses and Viral Vectors

Replication-incompetent LCMV-based vectors encoding HBV core and envelope proteins (rLCMV-core/env) were generated, grown and titrated as previously described (A. P. Bénéchet, G. D. Simone, P. D. Lucia, F. Cilenti, G. Barbiera, N. L. Bert, V. Fumagalli, E. Lusito, F. Moalli, V. Bianchessi, F. Andreata, P. Zordan, E. Bono, L. Giustini, W. V. Bonilla, C. Bleriot, K. Kunasegaran, G. Gonzalez-Aseguinolaza, D. D. Pinschewer, P. T. F. Kennedy, L. Naldini, M. Kuka, F. Ginhoux, A. Cantore, A. Bertoletti, R. Ostuni, L. G. Guidotti, M. Iannacone, Dynamics and genomic landscape of CD8+ T cells undergoing hepatic priming. Nature. 574, 200-205 (2019)). Mice were injected intravenously (i.v.) with 2.5×10⁵ infectious units of rLCMV vector 4 h before CD8⁺ T cell injection. All infectious work was performed in designated BSL-2 or BSL-3 workspaces, in accordance with institutional guidelines.

### Naive T Cell Isolation, Adoptive Transfer and In Vivo Treatments

Mice were adoptively transferred with 5×10⁶ or 1×10⁶ naive HBV-specific naive CD8⁺ TCR transgenic T cells isolated from the spleens of Cor93 and/or Env28 TCR transgenic mice, as described (Bénéchet et al. 2019). IL-2/anti-IL-2 complexes (IL-2c) were prepared by incubating 1.5 μg of rIL-2 (402 ML/CF, R&D Systems #402 ML/CF) with 50 μg anti-IL-2 mAb (clone S4B6-1, BioXcell) per mouse, as previously described (O. Boyman, M. Kovar, M. P. Rubinstein, C. D. Surh, J. Sprent, Selective Stimulation of T Cell Subsets with Antibody-Cytokine Immune Complexes. Science. 311, 1924-1927 (2006)). Mice were injected with IL-2c intraperitoneally (i.p.) one day after T cell transfer, unless otherwise indicated. In indicated experiments, naive CD8⁺ T cells from the spleens of Cor93 TCR transgenic mice were differentiated in vitro for 7-9 days into effector cells prior to adoptive transfer (1×10⁷ cells), as described (Bénéchet et al. 2019 and L. G. Guidotti, D. Inverso, L. Sironi, P. D. Lucia, J. Fioravanti, L. Ganzer, A. Fiocchi, M. Vacca, R. Aiolfi, S. Sammicheli, M. Mainetti, T. Cataudella, A. Raimondi, G. Gonzalez-Aseguinolaza, U. Protzer, Z. M. Ruggeri, F. V. Chisari, M. Isogawa, G. Sitia, M. Iannacone, Immunosurveillance of the liver by intravascular effector CD8(+) T cells. Cell. 161, 486-500 (2015); L. G. Guidotti, D. Inverso, L. Sironi, P. D. Lucia, J. Fioravanti, L. Ganzer, A. Fiocchi, M., Vacca, R. Aiolfi, S. Sammicheli, M. Mainetti, T. Cataudella, A. Raimondi, G. Gonzalez-Aseguinolaza, U. Protzer, Z. M. Ruggeri, F. V. Chisari, M. Isogawa, G. Sitia, M. Iannacone, Immunosurveillance of the liver by intravascular effector CD8(+) T cells. Cell. 161, 486-500 (2015)). In indicated experiments, Kupffer cells (KCs) were depleted by intravenous injection of 200 μl of clodronate-containing liposomes (Liposoma) 2 days prior to T cell injection, as described (Bénéchet et al. 2019). In indicated experiments, mice were injected i.p. with 200 μg of anti-Ly6G depleting antibody (clone 1A8) one day before and one day after T cell transfer. In indicated experiments, mice were injected intravenously (i.v.) with 200 μg of anti-Gr1 depleting antibody (clone RB6-8C5) every 48 h starting from 3 days before T cell transfer. In indicated experiments, mice were injected i.p. with 250 μg of anti-GM-CSF antibody (clone 22E9) every 48 h starting one day before T cell transfer. In indicated experiments, C57BL/6 or MUP-core mice were lethally irradiated and reconstituted for at least 8 weeks with BM from CD11c-DTR mice; dendritic cells were subsequently depleted by injecting i.p. 20 ng per gram of mouse of diphtheria toxin (Millipore) every 48 h starting from 3 days before T cell transfer. In indicated experiments, MUP-core mice were lethally irradiated and reconstituted for at least 8 weeks with BM from C57BL/6 WT or TAP1^{-/-} mice. To achieve full reconstitution of Kupffer cells from donor-derived BM, mice were injected with 200 μl of clodronate-containing liposomes 28 and 31 days after BM injection. In indicated experiments, MUP-core mice were lethally irradiated and reconstituted for at least 8 weeks with BM from Cdh5^{CreERT2}; Rosa26^{iDTR}; Rosa26^{tdTomato}; CX3CR1^{GFP} mice. To achieve full reconstitution of Kupffer cells from donor-derived BM, mice were injected with 200 μl of clodronate-containing liposomes 28 and 31 days after BM injection. To induce the expression of the Cre recombinase, mice were treated with 5 mg of Tamoxifen (Sigma) by oral gavage in 200 μl of corn oil one week before further manipulations. KC2 were depleted subsequently by injecting i.p. 20 ng per gram of mouse of diphtheria toxin (Millipore) 3 days and 1 day prior to T cell transfer.

### Cell Isolation and Flow Cytometry

Single-cell suspensions of liver, spleen and blood were generated as described (Bénéchet et al. 2019). Kupffer cell isolation was performed as described (Bénéchet et al. 2019). All flow cytometry stainings of surface-expressed and intracellular molecules were performed as described (M. D. Giovanni, V. Cutillo, A. Giladi, E. Sala, C. G. Maganuco, C. Medaglia, P. D. Lucia, E. Bono, C. Cristofani, E. Consolo, L. Giustini, A. Fiore, S. Eickhoff, W. Kastenmüller, I. Amit, M. Kuka, M. Iannacone, Spatiotemporal regulation of type I interferon expression determines the antiviral polarization of CD4+ T cells. Nat lmmunol. 21, 321-330 (2020)). Cell viability was assessed by staining with Viobility™ 405/520 fixable dye (Miltenyi) or DAPI. Abs used included: anti-CD3 (clone: 145-2C11, Cat #562286, BD Biosciences), anti-CD11b (clone: M1/70, Cat #101239), anti-CD19 (clone: 1D3, Cat #562291 BD Biosciences), anti-CD25 (clone: PC61, Cat #102015), anti-CD31 (clone: 390, Cat #102427), anti-CD45 (clone: 30-F11, Cat #564279 BD Biosciences), anti-CD64 (clone: X54-5/7.1, Cat #139311), anti-F4/80 (clone: BM8, Cat #123117), anti-I-A/I-E (clone: M5/114.15.2, Cat #107622), anti-TIM4 (clone: RTM4-54 Cat #130010), anti-TIM4 (polyclonal, Cat #orb103599 Biorbyt), anti-CD69 (clone: H1.2F3, Cat #104517), anti-CD45.1 (clone: A20, Cat #110716), anti-IFN-g (clone: XMG1.2, Cat #557735 BD Biosciences), anti-CD11c (clone: N418, Cat #117308), anti-1-Ab (clone: AF6-120.1, Cat #116420), anti-Stat5 pY694 (clone: 47, Cat #612599 BD Biosciences), anti-Foxp3 (clone FJK-16s, Cat #25-5773-82 Thermofisher), anti-CD122 (clone TM-B1 Cat #123210), anti-CD132 (clone TUgm2 Cat #132306), anti-CD40 (clone 3/23 Cat #558695 BD Biosciences), anti-CD80 (clone 1610A1 Cat #553769 BD Biosciences), anti-H2-K^{b} (clone AF6-88.5 Cat #742861 BD Biosciences), anti-ESAM (clone 1G8/ESAM, Cat #136203), anti-CD206 (clone C068C2, Cat #141712), anti-Ly6G (clone 1A8, Cat #562700 BD Biosciences), anti-Ly6C (clone HK1.4, Cat #128008), anti-CD49b (clone DX5, Cat #562453 BD Biosciences). All Abs were purchased from BioLegend, unless otherwise indicated. Recombinant dimeric H-2L^{d}:Ig and H-2K^{b}:Ig fusion proteins (BD Biosciences) complexed with peptides derived from HBsAg (Env28-39) or from HBcAg (Cor93-100), respectively, were prepared according to the manufacturer's instructions. Dimer staining was performed as described (M. Iannacone, G. Sitia, M. Isogawa, P. Marchese, M. G. Castro, P. R. Lowenstein, F. V. Chisari, Z. M. Ruggeri, L. G. Guidotti, Platelets mediate cytotoxic T lymphocyte-induced liver damage. Nat Med. 11, 1167-1169 (2005)). Flow cytometry staining for phosphorylated STAT5 was performed using Phosflow™ Perm Buffer III (Cat #558050, BD Bioscience), following the manufacturer's instructions. All flow cytometry analyses were performed in FACS buffer containing PBS with 2 mM EDTA and 2% FBS on a FACS CANTO or CytoFLEX LX (Beckman Coulter) and analyzed with FlowJo software (Treestar).

### Cell Purification

For the experiment described in FIG. 2, KCs were sorted from liver non-parenchymal cells as live, lineage negative (CD3, CD19, Ly6G, CD49b), CD45⁺, CD11b^{int}, F4/80⁺, CD64⁺, MHCII⁺, TIM4⁺ cells. For the experiment described in FIG. 3, KCs were sorted from liver nonparenchymal cells as live, CD45⁺, CD11b^{int}, F4/80⁺, MHCII⁺, TIM4⁺ cells. Among total KCs, KC1 were sorted as CD206⁻ ESAM⁻ cells and KC2 as CD206⁺, ESAM⁺ cells. Total KCs, KC1 and KC2 were FACS-sorted with a 100 μm nozzle at 4° C. on a FACSAria Fusion (BD) cell sorter in a buffer containing PBS with 2% FBS. Cells were always at least 98% pure. In indicated experiments, F4/80⁺ cells were purified from liver non-parenchymal cells by positive immunomagnetic separation (Miltenyi Biotec, #130-110-443), according to the manufacturer's instructions.

### RNA Purification and RNA-Seq Library Preparation

FACS-sorted KCs, KC1 and KC2 were lysed in ReliaPrep™ RNA Cell Miniprep System (Promega #Z6011) and total RNA was isolated following manual extraction. DNA digestion was performed with TURBO DNA-free™ Kit (Invitrogen #AM1907). RNA was quantified with Qubit™ RNA HS Assay Kit (Invitrogen #Q32852) and analysis of its integrity was assessed with Agilent RNA 6000 Pico Kit (Agilent #5067-1513) on a Bioanalyser instrument. 6 RNA samples of sorted KC1 and KC2, were processed with the “SMART-seq Ultra Low Input 48” library protocol in order to obtain 30.0M clusters of fragments of 1×100 nt of length through NovaSeq 6000 SP Reagent Kit (100 cycles). Raw reads were aligned to mouse genome build GRCm38 using STAR aligner (A. Dobin, T. R. Gingeras, Curr Protoc Bioinform, in press, doi:10.1002/0471250953.bi1114s51). Read counts per gene were then calculated using featureCounts (part of the R subread package) based on GENCODE gene annotation version M16. Read counts subject to Log 2 transformed transcripts per million (log 2 TPM) normalization were produced to account for transcript length and the total number of reads. Only genes with a TPM value higher than 1 in 3 samples or more were considered for following analysis. Differentially Expressed Genes (DEGs) between KC2 and KC1 samples were identified by generating a linear model using LIMMA R package (M. E. Ritchie, B. Phipson, D. Wu, Y. Hu, C. W. Law, W. Shi, G. K. Smyth, limma powers differential expression analyses for RNA-sequencing and microarray studies. Nucleic Acids Res. 43, e47 (2015)). Only DEGs with an adjusted P value<0.05 (using Benjamini Hochberg method) and a |log FC|>1 were selected for further analysis.

### RNA-Seq Transcriptome Analysis

Read counts were subject to log 2 TPM normalization, to account for transcript length and library size. For total KCs, only genes with a TPM value higher than 1 in at least 4 samples were considered for following analysis. For KC2 and KC1 populations, only genes with a TPM value higher than 1 in 3 samples were considered. Differentially Expressed Genes (DEGs) between samples treated with IL-2c and PBS were identified by generating a linear model using LIMMA R package (Ritchie et al. 2015). Only DEGs with an adjusted P value<0.05 (using Benjamini Hochberg correction method) were selected for further analysis. To identify Differentially Expressed Genes (DEGs) between KC2 in contrast to KC1, read counts were normalized with the Trimmed Mean of M-values (TMM) method using calcNormFactors function (from edgeR R package (M. D. Robinson, D. J. McCarthy, G. K. Smyth, edgeR: a Bioconductor package for differential expression analysis of digital gene expression data. Bioinformatics. 26, 139-140 (2009)) and transformed using the voomWithQualityWeights function before fitting a linear model using the LIMMA R package. Only DEGs with an adjusted P value<0.05 (using Benjamini-Hochberg correction method) and a |log FC|>1, were selected for further analysis.

### Functional Enrichment Analysis

Of the 4073 significant (FDR<0.05) identified DEGs between control (PBS) and treated (IL-2c) samples, 1515 were up-regulated and 2558 were down-regulated. Those were subject to a functional enrichment analysis using the EnrichR R package (M. V. Kuleshov, M. R. Jones, A. D. Rouillard, N. F. Fernandez, Q. Duan, Z. Wang, S. Koplev, S. L. Jenkins, K. M. Jagodnik, A. Lachmann, M. G. McDermott, C. D. Monteiro, G. W. Gundersen, A. Ma'ayan, Enrichr: a comprehensive gene set enrichment analysis web server 2016 update. Nucleic Acids Res. 44, W90-7 (2016)). Both the up- and the downregulated DEGs were checked for any biological signature enrichment in both the Gene Ontology Biological Process Database (2018) and the Kyoto Encyclopedia of Genes and Genomes for Mouse (2019). After merging the results for the two databases, 858 significant (FDR<0.05) Terms were identified, of which 428 were derived from the up-regulated DEGs and 430 from the down-regulated ones. In order to select the top enriched terms, only those with a high Combined Score (−log(p-value)*Odds Ratio) were considered. Based on the distribution of the Combined Score in the up-regulated terms and in the down-regulated ones, a threshold of 100 was chosen for the former, while a threshold of 30 for the latter.

### Clustering of Up-Regulated Terms

For visualization and analysis, both up-regulated and down-regulated terms were subject to a clustering algorithm, in order to identify the most prominent biological signatures. Briefly, a Jaccard Index Similarity score was calculated for each pair set of terms, based on the DEGs annotated for each term, using an in-house developed script. Next, terms were clustered using a hierarchical clustering method, using as distance measure the Pearson correlation between the calculated Jaccard Index Similarity scores. An arbitrary number of clusters was selected and manually annotated based on the terms present. To visualize the result, the pheatmap R package was used.

### Radar Plots Visualization

Radar plots were generated using the fmsb R package. Different sets of genes were selected based on literature analysis, defining different biological processes. For each category, the mean TPM expression for each gene within samples (separately for control and treated samples) was calculated. Next, the mean between all the genes belonging to a category was calculated and used as the value to represent the dimension in the radar plot.

### Network Plot Visualization

Network plot (FIG. 9) was built using Cytoscape software (V 3.8.0 for MacOS). Briefly, starting from EnrichR tables, a matrix defining every pair of term-gene was generated, and used as a node list input for Cytoscape.

### Gene Set Enrichment Analysis

Gene Set Enrichment Analysis (GSEA) was performed using the GseaPreranked Java tool (A. Subramanian, P. Tamayo, V. K. Mootha, S. Mukherjee, B. L. Ebert, M. A. Gillette, A. Paulovich, S. L. Pomeroy, T. R. Golub, E. S. Lander, J. P. Mesirov, Gene set enrichment analysis: A knowledge-based approach for interpreting genome-wide expression profiles. Proc National Acad Sci. 102, 15545-15550 (2005)) using pre-ranked Log 2 fold changes between KC2 and KC1 populations in expressed genes. HALLMARK_IL2_STAT5_SIGNALING Gene Set contained in MsigDB (Broad Institute) (A. Liberzon, C. Birger, H. Thorvaldsdóttir, M. Ghandi, J. P. Mesirov, P. Tamayo, The Molecular Signatures Database Hallmark Gene Set Collection. Cell Syst. 1, 417-425 (2015)), Version 6. Since the gene set is based on human genes, mouse orthologs in humans where identified using the homologene R package (https://CRAN.Rproject.org/package=homologene).

### Western Blot Analysis

Western blot on plated KCs was performed as described (P. Zordan, M. Cominelli, F. Cascino, E. Tratta, P. L. Poliani, R. Galli, Tuberous sclerosis complex—associated CNS abnormalities depend on hyperactivation of mTORC1 and Akt. J Clin Invest. 128, 1688-1706 (2018)). Primary Abs include anti-STAT5 and anti-pSTAT5 (Tyr694) (rabbit; Cell Signaling Technology #8215) and β-actin (polyclonal; Abcam ab228001). As secondary Ab horseradish peroxidase-conjugated goat antirabbit IgG (Jackson ImmunoResearch, Cat #111-035-003) was used. Reactive proteins were visualized using a Clarity Western ECL substrate kit (Bio-Rad), and exposure was performed using UVltec (Cambridge MINI HD, Eppendorf). Images were acquired by NineAlliance software. Each lane corresponds to a different mouse.

### Confocal Immunofluorescence Histology and Histochemistry

Confocal microscopy analysis of livers was performed as described (Guidotti et al. 2015). For confocal images of KC1 and KC2, C57BL/6 mice were injected i.v. with 2 μg F4/80 Alexa flour 488 (BioLegend #123120) and 2 μg CD206-APC (BioLegend 141708) 10 minutes before harvesting the liver. The liver was fixed overnight in PBS with 4% paraformaldehyde and subsequently incubated for 24 h in PBS with 30% sucrose. Next, liver lobes were embedded in O.C.T (Killik Bio-Optica 05-9801) and cut at −14° C. into 60 μm thick sections with a cryostat. Sections were blocked for 15 min with blocking buffer (PBS, 0.5% BSA, 0.3% Triton) and stained for 1 h at room temperature (RT) with anti-CD38 Alexa Fluor 594 (BioLegend #102725) in wash/stain buffer (PBS, 0.2% BSA, 0.1% triton). Sections were then washed twice for 5 min, stained with DAPI (Sigma 28718-90-3) for 5 min, washed again and mounted for imaging with FluorSave™ Reagent (Millipore 345789-20ML). For additional confocal imaging, the following primary Abs were used for staining: anti-CD45.1 (110702, BioLegend), anti-F4/80 (BM8, Invitrogen), antill Lyve-1 (NB600-1008, Novus Biological), anti-CD38 (102702, BioLegend). The following secondary Abs were used for staining: Alexa Fluor 488-, Alexa Fluor 514-, Alexa Fluor 568-, or Alexa Fluor 647-conjugated anti-rabbit or anti-rat IgG (Life Technologies). Image acquisition was performed with a 63× oil-immersion or 20× objective on an SP5 or SP8 confocal microscope (Leica Microsystem). To minimize fluorophore spectral spillover, the Leica sequential laser excitation and detection modality was used.

### Biochemical Analyses

The extent of hepatocellular injury was monitored by measuring serum alanine aminotransferase (sALT) activity at multiple time points after treatment, as previously described (Guidotti et al. 2015). Serum HBeAg was measured by enzyme-linked immunosorbent assays (ELISA), as previously described (Guidotti et al. 2015). Blood cell counts were measured by Vet abc™ (scil).

### Statistical Analyses

Results are expressed as mean±s.e.m. All statistical analyses were performed in Prism (GraphPad Software), and details are provided in the figure legends. Comparisons are not statistically significant unless indicated.

### Example 2

### Results And Discussion

Further to our finding that IL-2-based strategies reverted dysfunctional CD8⁺ T cell response to intrahepatic antigen presentation, we evaluated whether IL-2-based treatment was affected by Group 1 ILC. To this end, HBV Tg mice were treated with a-NK1.1 depleting antibody prior to the adoptive transfer of Cor93-naïve T cell (Cor93-T_{N}). One day after transfer, selected mice received recombinant IL-2 coupled with anti-IL-2 antibodies (IL-2c) (O. Boyman, M. Kovar, M. P. Rubinstein, C. D. Surh, J. Sprent, Selective Stimulation of T Cell Subsets with Antibody-Cytokine Immune Complexes. Science. 311, 1924-1927 (2006); A. P. Bénéchet, G. D. Simone, P. D. Lucia, F. Cilenti, G. Barbiera, N. L. Bert, V. Fumagalli, E. Lusito, F. Moalli, V. Bianchessi, F. Andreata, P. Zordan, E. Bono, L. Giustini, W. V. Bonilla, C. Bleriot, K. Kunasegaran, G. Gonzalez-Aseguinolaza, D. D. Pinschewer, P. T. F. Kennedy, L. Naldini, M. Kuka, F. Ginhoux, A. Cantore, A. Bertoletti, R. Ostuni, L. G. Guidotti, M. Iannacone, Dynamics and genomic landscape of CD8+ T cells undergoing hepatic priming. Nature. 574, 200-205 (2019)) (FIG. 17A). As shown in FIG. 17B, IL-2c administration sustained both NK cell and ILC1 expansion, in line with results obtained upon T_{E} cell transfer. The Cor93-T_{N} cell dysfunctionality upon hepatocellular antigen recognition was not reverted by Group 1 ILC depletion, as assessed by the little or no-production of IFN-γ (FIG. 17D) and the absence of cytotoxic activity (FIG. 17E). Of note, Group 1 ILC depletion reinforced the capacity of IL-2c to promote the expansion (FIG. 17C) and differentiation of Cor93-T cells into IFN-γ producing (Figure D) and cytotoxic effector cells (FIG. 17E). Thus, these results confirm that Group 1 ILCs consume IL-2 when this cytokine is available locally, that could derive either by T_{E} cell-mediated IL-2 release or by the external administration of IL-2c.

### Materials and Methods

### Mouse Model

HBV replication-competent transgenic mice (HBV Tg, lineage 1.3.32, inbred C57BL/6, H-2^{b}) express all of the HBV antigens and replicate HBV in the liver at high levels without any evidence of cytopathology (L. G. Guidotti, B. Matzke, H. Schaller, F. V. Chisari, High-level hepatitis B virus replication in transgenic mice. Journal of Virology. 69, 6158-6169 (1995)).

Cor93 T cell receptor (TCR) transgenic mice (lineage BC10.3, inbred CD45.1), in which >98% of the splenic CD8⁺ T cells recognize a K^{b}-restricted epitope located between residues 93-100 in the HBV core protein (MGLKFRQL) (M. Isogawa, J. Chung, Y. Murata, K. Kakimi, F. V. Chisari, CD40 activation rescues antiviral CD8⁺ T cells from PD-1-mediated exhaustion. PLoS Pathogens. 9, e1003490 (2013)).

### In Vivo Treatment

Mice were injected intravenously with 1×10⁶ HBV-specific naïve CD8⁺ TCR transgenic T cells isolated from the spleens of Cor93 TCR transgenic mice, as previously described (A. P. Bénéchet, G. D. Simone, P. D. Lucia, F. Cilenti, G. Barbiera, N. L. Bert, V. Fumagalli, E. Lusito, F. Moalli, V. Bianchessi, F. Andreata, P. Zordan, E. Bono, L. Giustini, W. V. Bonilla, C. Bleriot, K. Kunasegaran, G. Gonzalez-Aseguinolaza, D. D. Pinschewer, P. T. F. Kennedy, L. Naldini, M. Kuka, F. Ginhoux, A. Cantore, A. Bertoletti, R. Ostuni, L. G. Guidotti, M. Iannacone, Dynamics and genomic landscape of CD8+ T cells undergoing hepatic priming. Nature. 574, 200-205 (2019)). Group 1 ILCs were depleted by injecting intravenously 100 μg/mouse of anti-NK1.1 depleting antibody (Bioxcell, #BE0036, clone PK136) prior to T cell transfer. The day after, mice were injected intraperitoneally with IL-2/anti-IL-2 complexes (IL-2c). IL-2c were prepared by mixing 1.5 μg of recombinant IL-2 (clone 402 ML/CF, R&D, #402-ML-100) with 50 μg anti-IL-2 monoclonal antibody (clone S4B6-1, BioXcell, #BE0043-1) per mouse, as previously described (A. P. Bénéchet, G. D. Simone, P. D. Lucia, F. Cilenti, G. Barbiera, N. L. Bert, V. Fumagalli, E. Lusito, F. Moalli, V. Bianchessi, F. Andreata, P. Zordan, E. Bono, L. Giustini, W. V. Bonilla, C. Bleriot, K. Kunasegaran, G. Gonzalez-Aseguinolaza, D. D. Pinschewer, P. T. F. Kennedy, L. Naldini, M. Kuka, F. Ginhoux, A. Cantore, A. Bertoletti, R. Ostuni, L. G. Guidotti, M. Iannacone, Dynamics and genomic landscape of CD8+ T cells undergoing hepatic priming. Nature. 574, 200-205 (2019); O. Boyman, M. Kovar, M. P. Rubinstein, C. D. Surh, J. Sprent, Selective Stimulation of T Cell Subsets with Antibody-Cytokine Immune Complexes. Science. 311, 1924-1927 (2006)).

### Cell Isolation And Flow Cytometry

A single cell suspension was prepared from the liver as previously described (Iannacone et al., 2005.). Briefly, for intrahepatic leukocyte (IHL) isolation, mouse livers were perfused with PBS via the inferior vena cava and pressed through a 70 μm. Total liver cells were digested with 10 ml RPMI 1640 containing 0.02% wt/vol Collagenase IV (Sigma, #C5138) and 0.002% (wt/vol) DNase I (Sigma, #D4263) for 40 minutes at 37° C. Cells were washed with RPMI 1640 and resuspended with 36% Percoll solution (Sigma, #P4937) and centrifuged for 20 minutes at 2000 rpm (low brake). IHLs were lysed with ACK and then counted using trypan blue dye. Single-cell suspensions were prepared from two liver lobes of known weight, and analysis of IHL population was performed by flow cytometry.

All flow cytometry staining of surface-expressed and intracellular molecules were performed as described (Benchet 2019). Cell viability was assessed by staining with Viobility™ 405/520 fixable dye (Miltenyi, Cat #130-109-814). Recombinant dimeric H-2L^{d}:Ig and H-2K^{b}:Ig fusion proteins (BD Biosciences) complexed with peptides derived from HBcAg (Cor93-100), were prepared according to the manufacturer's instructions.

The following antibodies were used:

| Antibody | Clone | Source and identifier |
|---|---|---|
| CD45 | 30-F11 | Biolegend #103113 |
| CD8 | 53-6.7 | Biolegend #100722 |
| | | Biolegend #100759 |
| | | BD Pharmingen #558106 |
| CD45.1 | A20 | Biolegend #110716 |
| | | BD Pharmingen #561235 |
| CD3 | 17A2 | Biolegend #100204 |
| | 145-2C11 | Biolegend #100330 |
| NKp46 | 29A1.4 | Biolegend #137604 |
| | | Biolegend #137618 |
| NK1.1 | PK136 | Biolegend #108722 |
| | | Biolegend #108717 |
| | 145-2C11 | BD Pharmingen #551114 |
| CD49a | HMalfa1 | Biolegend #142605 |
| CD49b | DX5 | Biolegend #108916 |
| hGrzB | GB11 | Biolegend #515406 |
| | QA16A02 | Biolegend #372207 |
| IFNg | XMG1.2 | Biolegend #505813 |
| | | BD Pharmingen #562333 |
| | | BD Pharmingen #554412 |

### Biochemical Analysis

The extent of hepatocellular injury was monitored by measuring serum alanine aminotransferase (sALT) activity at multiple time points after treatment, as previously described (L. G. Guidotti, D. Inverso, L. Sironi, P. D. Lucia, J. Fioravanti, L. Ganzer, A. Fiocchi, M. Vacca, R. Aiolfi, S. Sammicheli, M. Mainetti, T. Cataudella, A. Raimondi, G. Gonzalez-Aseguinolaza, U. Protzer, Z. M. Ruggeri, F. V. Chisari, M. Isogawa, G. Sitia, M. Iannacone, Immunosurveillance of the liver by intravascular effector CD8(+) T cells. Cell. 161, 486-500 (2015)).

### Example 3

To assess the effect of OX-40/OX-40L axis perturbation in HBV-specific naïve CD8⁺ T cell undergoing hepatocellular priming, antibodies that have been shown to be agonists of OX40 (which is transiently expressed on T cells undergone TCR engagement) or to block the activity of OX40L (which is expressed on APCs), were injected in MU P-core mice adoptively transferred with TCR-transgenic naïve CD8+ T cells specific for an epitope of the HBV-core protein (FIG. 18A). As shown, mice that have been treated with OX40 agonist showed CD8-mediated immunopathology (FIGS. 18B and F) as a result of an efficient CD8 T cell expansion (FIG. 18C) and differentiation in IFNγ-producing effector CD8+ T cells. OX40 agonist rescued the intraparenchymal distribution of Ag-specific T cell as they passed from periportal accumulation to be scattered throughout the liver parenchyma (FIG. 18E).

### Material and Methods

### Mice

MUP-core transgenic mice (lineage MUP-core 50 [MC50], inbred C57BL/6, H-2b), that express the HBV core protein in 100% of the hepatocytes under the transcriptional control of the mouse major urinary protein (MUP) promoter, have been previously described (L. G. Guidotti, V. Martinez, Y. T. Loh, C. E. Rogler, F. V. Chisari, Hepatitis B virus nucleocapsid particles do not cross the hepatocyte nuclear membrane in transgenic mice. Journal of Virology. 68, 5469-5475 (1994)). Cor93 TCR transgenic mice (lineage BC10.3, inbred CD45.1), in which >98% of the splenic CD8+ T cells recognize a Kb-restricted epitope located between residues 93-100 in the HBV core protein (MGLKFRQL), have been previously described (M. Isogawa, J. Chung, Y. Murata, K. Kakimi, F. V. Chisari, CD40 activation rescues antiviral CD8⁺ T cells from PD-1-mediated exhaustion. PLoS Pathogens. 9, e1003490 (2013)).

### Naïve T Cell Isolation, Adoptive Transfer and In Vivo Treatments

Mice were adoptively transferred with 1×10⁶ naïve HBV-specific naïve CD8⁺ TCR transgenic T cells isolated from the spleens of Cor93 TCR transgenic mice as described (A. P. Bénéchet, G. D. Simone, P. D. Lucia, F. Cilenti, G. Barbiera, N. L. Bert, V. Fumagalli, E. Lusito, F. Moalli, V. Bianchessi, F. Andreata, P. Zordan, E. Bono, L. Giustini, W. V. Bonilla, C. Bleriot, K. Kunasegaran, G. Gonzalez-Aseguinolaza, D. D. Pinschewer, P. T. F. Kennedy, L. Naldini, M. Kuka, F. Ginhoux, A. Cantore, A. Bertoletti, R. Ostuni, L. G. Guidotti, M. Iannacone, Dynamics and genomic landscape of CD8+ T cells undergoing hepatic priming. Nature. 574, 200-205 (2019)). Indicated mice were injected intra peritoneally (i.p.) with 100 μg of anti-mouse OX40 agonist antibody (clone OX-86, BioXcell #BE0031) or with 100 μg of anti-mouse OX40L blocking antibody (clone RM134L, BioXcell #BE0033-1) every 48 hours as been previously described (J. Publicover, et al. Sci Transl Med. 2018).

### Intrahepatic Leukocytes Cell Isolation and Flow Cytometry

Single-cell suspensions of liver was generated as described (A. P. Bénéchet, G. D. Simone, P. D. Lucia, F. Cilenti, G. Barbiera, N. L. Bert, V. Fumagalli, E. Lusito, F. Moalli, V. Bianchessi, F. Andreata, P. Zordan, E. Bono, L. Giustini, W. V. Bonilla, C. Bleriot, K. Kunasegaran, G. Gonzalez-Aseguinolaza, D. D. Pinschewer, P. T. F. Kennedy, L. Naldini, M. Kuka, F. Ginhoux, A. Cantore, A. Bertoletti, R. Ostuni, L. G. Guidotti, M. Iannacone, Dynamics and genomic landscape of CD8+ T cells undergoing hepatic priming. Nature. 574, 200-205 (2019)). Cell viability was assessed by staining with Viobility™ 405/520 fixable dye (Miltenyi) and antibodies used included: anti-CD3 (clone: 145-2C11, Cat #562286, BD Biosciences), anti-CD8 (clone: 53-67, Cat #558106, BD Biosciences) anti-CD45 (clone: 30-F11, Cat #564279 BD Biosciences), anti-CD69 (clone: H1.2F3, Cat #104517), anti-CD45.1 (clone: A20, Cat #110716), anti-IFN-g (clone: XMG1.2, Cat #557735 BD Biosciences).

### Confocal Immunofluorescence Histology and Histochemistry

Confocal microscopy analysis of livers was performed as described (L. G. Guidotti, D. Inverso, L. Sironi, P. D. Lucia, J. Fioravanti, L. Ganzer, A. Fiocchi, M. Vacca, R. Aiolfi, S. Sammicheli, M. Mainetti, T. Cataudella, A. Raimondi, G. Gonzalez-Aseguinolaza, U. Protzer, Z. M. Ruggeri, F. V. Chisari, M. Isogawa, G. Sitia, M. Iannacone, Immunosurveillance of the liver by intravascular effector CD8(+) T cells. Cell. 161, 486-500 (2015)). Briefly, the liver was fixed overnight in PBS with 4% paraformaldehyde and subsequently incubated for 24 h in PBS with 30% sucrose. Next, liver lobes were embedded in O.C.T (Killik Bio-Optica 05-9801) and cut at −14° C. into 60 μm thick sections with a cryostat. Sections were blocked for 15 min with blocking buffer (PBS, 0.5% BSA, 0.3% Triton) and stained for 1 h at room temperature (RT) with anti-CD38 Alexa Fluor 594 (BioLegend #102725) in wash/stain buffer (PBS, 0.2% BSA, 0.1% triton). Sections were then washed twice for 5 min, stained with DAPI (Sigma 28718-90-3) for 5 min, washed again and mounted for imaging with FluorSave™ Reagent (Millipore 345789-20ML). For detection of intrahepatic Cor93 TCR transgenic T cell, anti-CD45.1 antibody (110702, BioLegend) was used. Image acquisition was performed with a 63× oil-immersion or 20× objective on an SP5 or SP8 confocal microscope (Leica Microsystem). To minimize fluorophore spectral spillover, the Leica sequential laser excitation and detection modality was used.

### Biochemical Analyses

The extent of hepatocellular injury was monitored by measuring serum alanine aminotransferase (sALT) activity at multiple time points after treatment, as previously described (L. G. Guidotti, D. Inverso, L. Sironi, P. D. Lucia, J. Fioravanti, L. Ganzer, A. Fiocchi, M. Vacca, R. Aiolfi, S. Sammicheli, M. Mainetti, T. Cataudella, A. Raimondi, G. Gonzalez-Aseguinolaza, U. Protzer, Z. M. Ruggeri, F. V. Chisari, M. Isogawa, G. Sitia, M. Iannacone, Immunosurveillance of the liver by intravascular effector CD8(+) T cells. Cell. 161, 486-500 (2015)).

### Statistical Analyses

Results are expressed as mean showing all points. All statistical analyses were performed in Prism (GraphPad Software), and details are provided in the figure legend. Comparisons are not statistically significant unless indicated.

All publications mentioned in the above specification are herein incorporated by reference. Various modifications and variations of the disclosed agents, products, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. An agent that increases the number of Kupffer cells in a subject, or a nucleotide sequence encoding therefor, for use in a method of therapy by increasing liver immune response.

2. The agent for use according to claim 1, wherein the agent is administered simultaneously, sequentially or separately in combination with an interleukin that binds to IL-2 receptor (IL-2R), or a nucleotide sequence encoding therefor.

3. An interleukin that binds to IL-2 receptor (IL-2R), or a nucleotide sequence encoding therefor, for use in a method of therapy by increasing liver immune response, wherein the interleukin is administered simultaneously, sequentially, or separately in combination with an agent that increases the number of Kupffer cells in a subject, or a nucleotide sequence encoding therefor.

4. The agent or interleukin for use according to any preceding claim, wherein the agent increases the number of Type 2 Kupffer cells (KC2) in the subject.

5. The agent or interleukin for use according to any preceding claim, wherein the method of therapy is treatment or prevention of a liver infection, and/or treatment or prevention of a primary or secondary liver tumour.

6. The agent or interleukin for use according to any preceding claim, wherein the agent is a granulocyte-macrophage colony-stimulating factor (GM-CSF) inhibitor.

7. The agent or interleukin for use according to any one of claims 2-6, wherein the interleukin is selected from the group consisting of IL-2, IL-7 or IL-15.

8. The agent or interleukin for use according to any preceding claim, wherein the agent, interleukin and/or nucleotide sequence(s) encoding therefor is adapted to be targeted to the liver.

9. The agent or interleukin for use according to any preceding claim, wherein the agent, interleukin and/or nucleotide sequence encoding therefor is comprised in a nanoparticle, optionally wherein the nanoparticle comprises a liver-specific ligand.

10. The agent or interleukin for use according to any preceding claim, wherein the nucleotide sequence(s) encoding the agent and/or interleukin is in the form of one or more vectors adapted for liver-specific expression of the nucleotide sequence.

11. The agent or interleukin for use according to any preceding claim, wherein the nucleotide sequence(s) encoding the agent and/or interleukin is operably linked to one or more expression control sequences for liver-specific expression

12. The agent or interleukin for use according to claim 10 or 11, wherein the one or more vector(s) comprises a liver-specific promoter and/or enhancer operably linked to the nucleotide sequence(s) encoding the agent and/or interleukin, optionally wherein the vector(s) comprises a hepatocyte-specific promoter and/or enhancer operably linked to the nucleotide sequence(s).

13. The agent or interleukin for use according to any preceding claim, wherein the agent and/or interleukin, or nucleotide sequence(s) encoding therefor, is administered as part of an adoptive T cell therapy.

14. The agent or interleukin for use according to any preceding claim, wherein the agent and/or interleukin, or nucleotide sequence(s) encoding therefor, is administered simultaneously, separately or sequentially with a population of T cells, optionally wherein the T cells express a chimeric antigen receptor (CAR) or a T cell receptor (TCR).

15. A product comprising: (a) an agent that increases the number of Kupffer cells in a subject, or a nucleotide sequence encoding therefor; and (b) an interleukin that binds to IL-2 receptor (IL-2R), or nucleotide sequence encoding therefor, optionally wherein the product is a kit or a composition.

16. A product comprising: (a) an agent that increases the number of Kupffer cells in a subject, or a nucleotide sequence encoding therefor; and (b) a population of T cells, optionally wherein the T cells express a chimeric antigen receptor (CAR) or a T cell receptor (TCR), optionally wherein the product is a kit or a composition.
